SEQUENCE LISTING (119 sequences)

SEQ ID NO: 1 - protein, 52 aa, Homo sapiens
YRQSMNNFQGLRSFGCRFGTCTVQKLAHQIYQFTDKDKDNVAPRSKISPQGY

SEQ ID NO: 2 - protein, 40 aa, Homo sapiens
SFGCRFGTCTVQKLAHQIYQFTDKDKDNVAPRSKISPQGY

SEQ ID NO: 3 - protein, 31 aa, Homo sapiens
TVQKLAHQIYQFTDKDKDNVAPRSKISPQGY

SEQ ID NO: 4 - protein, 16 aa, Homo sapiens
DKDNVAPRSKISPQGY

SEQ ID NO: 5 - protein, 53 aa, Homo sapiens
HSGPRRTQAQLLRVGCVLGTCQVQNLSHRLWQLMGPAGRQDSAPVDPSSPHSY

SEQ ID NO: 6 - protein, 47 aa, Homo sapiens
TQAQLLRVGCVLGTCQVQNLSHRLWQLMGPAGRQDSAPVDPSSPHSY

SEQ ID NO: 7 - protein, 40 aa, Homo sapiens
VGCVLGTCQVQNLSHRLWQLMGPAGRQDSAPVDPSSPHSY

SEQ ID NO: 8 - protein, 31 aa, Homo sapiens
VQNLSHRLWQLMGPAGRQDSAPVDPSSPHSY

SEQ ID NO: 9 - protein, 37 aa, Homo sapiens
ACDTATCVTHRLAGLLSRSGGVVKNNFVPTNVGSKAF

SEQ ID NO: 10 - protein, 30 aa, Homo sapiens
VTHRLAGLLSRSGGVVKNNFVPTNVGSKAF

SEQ ID NO: 11 - protein, 11 aa, Homo sapiens
FVPTNVGSKAF

SEQ ID NO: 12 - protein, 37 aa, Homo sapiens
ACNTATCVTHRLAGLLSRSGGMVKSNFVPTNVGSKAF

SEQ ID NO: 13 - protein, 37 aa, Homo sapiens [sequence omitted]

SEQ ID NO: 14 - protein, 30 aa, Homo sapiens
ATQRLANFLVHSSNNFGAILSSTNVGSNTY

SEQ ID NO: 15 - protein, 32 aa, Homo sapiens
CGNLSTCMLGTYTQDFNKFHTFPQTAIGVGAP

SEQ ID NO: 16 - protein, 25 aa, Homo sapiens
MLGTYTQDFNKFHTFPQTAIGVGAP

SEQ ID NO: 17 - protein, 32 aa, Homo sapiens
CSNLSTCVLGKLSQELHKLQTYPRTNTGSGTP

SEQ ID NO: 18 - protein, 25 aa, Homo sapiens
VLGKLSQELHKLQTYPRTNTGSGTP

SEQ ID NO: 19 - protein, 31 aa, synthetic construct
  VARIANT 1: note = X1 is V, wherein the V optionally comprises a blocking group
  VARIANT 7: note = X7 is R, K, Q, L, A, or S
  VARIANT 17: note = X17 is R, K, A, S, or N
  VARIANT 29: note = X29 is W, H, Q, Y, F, R, K, I, L, or M
  VARIANT 31: note = X31 is Y or F, wherein the Y or F comprises a second blocking group
XQNLSHXLWQLMGPAGXQDSAPVDPSSPXSX

SEQ ID NO: 20 - protein, 31 aa, synthetic construct
VQNLSHRLWQLMGPAGRQDSAPVDPSSPWSY

SEQ ID NO: 21 - protein, 31 aa, synthetic construct
  VARIANT 1: note = X1 is V, T, or L, and wherein the V, T, or L optionally comprises a first blocking group
  VARIANT 2: note = X2 is Q, H, or G
  VARIANT 3: note = X3 is N, K, R, or T
  VARIANT 5: note = X5 is S, A, or T
  VARIANT 6: note = X6 is H, G, N, or Q
  VARIANT 24: note = X24 is S, W, F, Y, or R
  VARIANT 25: note = X25 is K, L, R, Q, Y, I, V, M, A, or G
  VARIANT 27: note = X27 is S, or G
  VARIANT 29: note = X29 is Q, W, Y, F, R, K, I, L, or M
  VARIANT 31: note = X31 is Y or F, and wherein the Y or F comprises a second blocking group
XXXLXXRLWQLMGPAGRDNVAPRXXIXPXGX

SEQ ID NO: 22 - protein, 31 aa, synthetic construct
VQNLSHRLWQLMGPAGRDNVAPRSKISPQGY

SEQ ID NO: 23 - protein, 31 aa, synthetic construct
VQNLSHRLWQLMGPAGRDNVAPRWKISPWGY

SEQ ID NO: 24 - protein, 31 aa, synthetic construct
VQNLSHRLWQLMGPAGRDNVAPRWKISPWGF

SEQ ID NO: 25 - protein, 31 aa, synthetic construct
VQNLSHRLWQLMGPAGRDNVAPRWLISPWGF

SEQ ID NO: 26 - protein, 32 aa, synthetic construct
  VARIANT 1: note = X1 is T, V, A or M, and wherein the T, V, A or M optionally comprises a first blocking group
  VARIANT 2: note = X2 is V, T, or L
  VARIANT 3: note = X3 is Q, H, or G
  VARIANT 4: note = X4 is K, N, R, or T
  VARIANT 6: note = X6 is A, S, or T
  VARIANT 7: note = X7 is H, N, G, or Q
  VARIANT 25: note = X25 is S, W, F, Y, or R
  VARIANT 26: note = X26 is K, L, R, Q, Y, I, V, M, A, or G
  VARIANT 28: note = X28 is S or G
  VARIANT 30: note = X30 is Q, W, F, Y, R, K, I, L, or M
  VARIANT 32: note = X32 is Y or F, and wherein the Y or F comprises a second blocking group
XXXXLXXRLWQLMGPAGRDNVAPRXXIXPXGX

SEQ ID NO: 27 - protein, 32 aa, synthetic construct
TVQKLAHRLWQLMGPAGRDNVAPRSKISPQGY

SEQ ID NO: 28 - protein, 32 aa, synthetic construct
TVQKLAHRLWQLMGPAGRDNVAPRWKISPWGY

SEQ ID NO: 29 - protein, 32 aa, synthetic construct
TVQKLAHRLWQLMGPAGRDNVAPRWKISPWGF

SEQ ID NO: 30 - protein, 32 aa, synthetic construct
TVQKLAHRLWQLMGPAGRDNVAPRWLISPWGF

SEQ ID NO: 31 - protein, 32 aa, synthetic construct
  VARIANT 1: note = X1 is V, and optionally comprises a first blocking group
  VARIANT 25: note = X25 is S, W, F, Y, or R
  VARIANT 26: note = X26 is K, L, R, Q, Y, I, V, M, A, or G
  VARIANT 28: note = X28 is S or G
  VARIANT 30: note = X30 is Q, W, F, Y, R, K, I, L, or M
  VARIANT 32: note = X32 is Y or F, and wherein the Y or F comprises a second blocking group
XTHRLAGRLWQLMGPAGRDNVAPRXXIXPXGX

SEQ ID NO: 32 - protein, 32 aa, synthetic construct
VTHRLAGRLWQLMGPAGRDNVAPRSKISPQGY

SEQ ID NO: 33 - protein, 32 aa, synthetic construct
  VARIANT 1: note = X1 is A, and optionally is linked to a first blocking group
  VARIANT 25: note = X25 is S, W, F, Y, or R
  VARIANT 26: note = X26 is K, L, R, Q, Y, I, V, M, A, or G
  VARIANT 28: note = X28 is S or G
  VARIANT 30: note = X30 is Q, W, F, Y, R, K, I, L, or M
  VARIANT 32: note = X32 is Y or F, and wherein the Y or F comprises a second blocking group
XTQRLANRLWQLMGPAGRDNVAPRXXIXPXGX

SEQ ID NO: 34 - protein, 32 aa, synthetic construct
ATQRLANRLWQLMGPAGRDNVAPRSKISPQGY

SEQ ID NO: 35 - protein, 32 aa, synthetic construct
  VARIANT 1: note = X1 is M, and optionally is linked to a first blocking group
  VARIANT 25: note = X25 is S, W, F, Y, or R
  VARIANT 26: note = X26 is K, L, R, Q, Y, I, V, M, A, or G
  VARIANT 28: note = X28 is S or G
  VARIANT 30: note = X30 is Q, W, F, Y, R, K, I, L, or M
  VARIANT 32: note = X32 is Y or F, and wherein the Y or F comprises a second blocking group
XLGTYTQRLWQLMGPAGRDNVAPRXXIXPXGX

SEQ ID NO: 36 - protein, 32 aa, synthetic construct
MLGTYTQRLWQLMGPAGRDNVAPRSKISPQGY

SEQ ID NO: 37 - protein, 32 aa, synthetic construct; identical to SEQ ID NO: 35
  VARIANT 1: note = X1 is V, and optionally is linked to a first blocking group
  VARIANT 25: note = X25 is S, W, F, Y, or R
  VARIANT 26: note = X26 is K, L, R, Q, Y, I, V, M, A, or G
  VARIANT 28: note = X28 is S or G
  VARIANT 30: note = X30 is Q, W, F, Y, R, K, I, L, or M
  VARIANT 32: note = X32 is Y or F, and wherein the Y or F comprises a second blocking group

SEQ ID NO: 38 - protein, 32 aa, synthetic construct
VLGTYTQRLWQLMGPAGRDNVAPRSKISPQGY

SEQ ID NO: 39 - protein, 53 aa, synthetic construct
YRQSMNNFQGLRSFGCRFGTCTVQNLSHRLWQLMGPAGRQDSAPVDPSSPHSY

SEQ ID NO: 40 - protein, 53 aa, synthetic construct
YRQSMNNFQGLRSFGCRFGTCTVQKLAHRLWQLMGPAGRDNVAPRSKISPQGY

SEQ ID NO: 41 - protein, 30 aa, synthetic construct
VTHRLAGLLSRSGGVVKNNFVPTDVGPWSF

SEQ ID NO: 42 - protein, 31 aa, synthetic construct
TVQKLAHQIYQFTDKDKDNVAPRSKIGPWGY

SEQ ID NO: 43 - protein, 41 aa, synthetic construct
SFGCRFGTCTVQKLAHQIYQFMGPAGRQDSAPVDPSSPHSY

SEQ ID NO: 44 - protein, 39 aa, synthetic construct
VGCVLGTCQVQNLSHRLWQLTDKDKDNVAPRSKISPQGY

SEQ ID NO: 45 - protein, 40 aa, synthetic construct
SFGCRFGTCTVQKLAHQIYQFTDKDKQDSAPVDPSSPHSY

SEQ ID NO: 46 - protein, 40 aa, synthetic construct
VGCVLGTCQVQNLSHRLWQLMGPAGRDNVAPRSKISPQGY

SEQ ID NO: 47 - protein, 51 aa, synthetic construct
SFGCRFGTCTVQKLAHQIYQFTDKDKRLWQLMGPAGRDNVAPRSKISPQGY

SEQ ID NO: 48 - protein, 39 aa, synthetic construct
VGCVLGTCQVQNLSHQIYQFTDKDKQDSAPVDPSSPHSY

SEQ ID NO: 49 - protein, 40 aa, synthetic construct
  MOD_RES 28: note = TAMRA
SFGCRFGTCTVQKLAHQIYQFTDKDKDKVAPRSKISPQGY

SEQ ID NO: 50 - protein, 40 aa, synthetic construct
  MOD_RES 28: note = TAMRA
VGCVLGTCQVQNLSHRLWQLMGPAGRQKSAPVDPSSPHSY

SEQ ID NO: 51 - protein, 41 aa, synthetic construct
SFGCRFGTCTVQKLAHRLWQLMGPAGRDNVAPRSKIGPWGY

SEQ ID NO: 52 - protein, 40 aa, synthetic construct
SFGCRFGTCTVQKLAHQIYQFTDKDKDNVAPRSKIGPWGY

SEQ ID NO: 53 - protein, 40 aa, synthetic construct
VGCVLGTCQVQNLSHRLWQLMGPAGRDNVAPRSKIGPWGY

SEQ ID NO: 54 - protein, 121 aa, Homo sapiens
GGTVKNYETAVQFCWNHYKDQMDPIEKDWCDWAMISRPYSTLRDCLEHFAELFDLGFPNPLAERIIFETHQIHFANCSLVQPTFSDPPEDVLIAMIIAPICLIPFLITLVVWRSKDSEAQA

SEQ ID NO: 55 - protein, 122 aa, Homo sapiens
GCNETGMLERLPLCGKAFADMMGKVDVWKWCNLSEFIVYYESFTNCTEMEANVVGCYWPNPLAQGFITGIHRQFFSNCTVDRVHLEDPPDEVLIPLIVIPVVLTVAMAGLVVWRSKRTDTLL

SEQ ID NO: 56 - protein, 40 aa, synthetic construct
VGCVLGTCQVQNLSHRLWQLMGPAGRDNVAPRWRIGPWGF

SEQ ID NO: 57 - protein, 31 aa, synthetic construct
VQNLSHRLWQLMGPAGRDNVAPRSKIGPWGY

SEQ ID NO: 58 - protein, 31 aa, synthetic construct
VQNLSHRLWQLMGPAGRDNVAPRWKIGPWGY

SEQ ID NO: 59 - protein, 31 aa, synthetic construct
VQNLSHRLWQLMGPAGRDNVAPRWLISPWGY

SEQ ID NO: 60 - protein, 31 aa, synthetic construct
VQNLSHRLWQLMGPAGRDNVAPRWRISPWGY

SEQ ID NO: 61 - protein, 31 aa, synthetic construct
VQNLSHRLWQLMGPAGRDNVAPRSLIGPWGY

SEQ ID NO: 62 - protein, 31 aa, synthetic construct
VQNLSHRLWQLMGPAGRDNVAPRSRIGPWGY

SEQ ID NO: 63 - protein, 31 aa, synthetic construct
VQNLSHRLWQLMGPAGRDNVAPRSKIGPWGF

SEQ ID NO: 64 - protein, 31 aa, synthetic construct
VQNLSHRLWQLMGPAGRDNVAPRWKIGPWGF

SEQ ID NO: 65 - protein, 31 aa, synthetic construct
VQNLSHRLWQLMGPAGRDNVAPRWLIGPWGY

SEQ ID NO: 66 - protein, 31 aa, synthetic construct
VQNLSHRLWQLMGPAGRDNVAPRWRIGPWGY

SEQ ID NO: 67 - protein, 31 aa, synthetic construct
VQNLSHRLWQLMGPAGRDNVAPRSLIGPWGF

SEQ ID NO: 68 - protein, 31 aa, synthetic construct
VQNLSHRLWQLMGPAGRDNVAPRSRIGPWGF

SEQ ID NO: 69 - protein, 31 aa, synthetic construct
VQNLSHRLWQLMGPAGRDNVAPRWRISPWGF

SEQ ID NO: 70 - protein, 31 aa, synthetic construct
VQNLSHRLWQLMGPAGRDNVAPRWLIGPWGF

SEQ ID NO: 71 - protein, 31 aa, synthetic construct
VQNLSHRLWQLMGPAGRDNVAPRWRIGPWGF

SEQ ID NO: 72 - protein, 32 aa, synthetic construct
TVQKLAHRLWQLMGPAGRDNVAPRSKIGPWGY

SEQ ID NO: 73 - protein, 32 aa, synthetic construct
TVQKLAHRLWQLMGPAGRDNVAPRSKIGPWGF

SEQ ID NO: 74 - protein, 32 aa, synthetic construct
TVQKLAHRLWQLMGPAGRDNVAPRWLISPWGY

SEQ ID NO: 75 - protein, 32 aa, synthetic construct
TVQKLAHRLWQLMGPAGRDNVAPRWRISPWGY

SEQ ID NO: 76 - protein, 32 aa, synthetic construct
TVQKLAHRLWQLMGPAGRDNVAPRWKIGPWGY

SEQ ID NO: 77 - protein, 32 aa, synthetic construct
TVQKLAHRLWQLMGPAGRDNVAPRSLIGPWGY

SEQ ID NO: 78 - protein, 32 aa, synthetic construct
TVQKLAHRLWQLMGPAGRDNVAPRSRIGPWGY

SEQ ID NO: 79 - protein, 32 aa, synthetic construct
TVQKLAHRLWQLMGPAGRDNVAPRWLIGPWGY

SEQ ID NO: 80 - protein, 32 aa, synthetic construct
TVQKLAHRLWQLMGPAGRDNVAPRWRIGPWGY

SEQ ID NO: 81 - protein, 32 aa, synthetic construct
TVQKLAHRLWQLMGPAGRDNVAPRSLIGPWGF

SEQ ID NO: 82 - protein, 32 aa, synthetic construct
TVQKLAHRLWQLMGPAGRDNVAPRSRIGPWGF

SEQ ID NO: 83 - protein, 32 aa, synthetic construct
TVQKLAHRLWQLMGPAGRDNVAPRWKIGPWGF

SEQ ID NO: 84 - protein, 32 aa, synthetic construct
TVQKLAHRLWQLMGPAGRDNVAPRWRISPWGF

SEQ ID NO: 85 - protein, 32 aa, synthetic construct
TVQKLAHRLWQLMGPAGRDNVAPRWLIGPWGF

SEQ ID NO: 86 - protein, 32 aa, synthetic construct
TVQKLAHRLWQLMGPAGRDNVAPRWRIGPWGF

SEQ ID NO: 87 - protein, 41 aa, synthetic construct
SFGCRFGTCTVQKLAHRLWQLMGPAGRDNVAPRWKISPWGY

SEQ ID NO: 88 - protein, 41 aa, synthetic construct
SFGCRFGTCTVQKLAHRLWQLMGPAGRDNVAPRSKIGPWGF

SEQ ID NO: 89 - protein, 41 aa, synthetic construct
SFGCRFGTCTVQKLAHRLWQLMGPAGRDNVAPRWLISPWGY

SEQ ID NO: 90 - protein, 41 aa, synthetic construct
SFGCRFGTCTVQKLAHRLWQLMGPAGRDNVAPRWRISPWGY

SEQ ID NO: 91 - protein, 41 aa, synthetic construct
SFGCRFGTCTVQKLAHRLWQLMGPAGRDNVAPRWKIGPWGY

SEQ ID NO: 92 - protein, 41 aa, synthetic construct
SFGCRFGTCTVQKLAHRLWQLMGPAGRDNVAPRWKISPWGF

SEQ ID NO: 93 - protein, 41 aa, synthetic construct
SFGCRFGTCTVQKLAHRLWQLMGPAGRDNVAPRSLIGPWGY

SEQ ID NO: 94 - protein, 41 aa, synthetic construct
SFGCRFGTCTVQKLAHRLWQLMGPAGRDNVAPRSRIGPWGY

SEQ ID NO: 95 - protein, 41 aa, synthetic construct
SFGCRFGTCTVQKLAHRLWQLMGPAGRDNVAPRWLIGPWGY

SEQ ID NO: 96 - protein, 41 aa, synthetic construct
SFGCRFGTCTVQKLAHRLWQLMGPAGRDNVAPRWRIGPWGY

SEQ ID NO: 97 - protein, 41 aa, synthetic construct
SFGCRFGTCTVQKLAHRLWQLMGPAGRDNVAPRSLIGPWGF

SEQ ID NO: 98 - protein, 41 aa, synthetic construct
SFGCRFGTCTVQKLAHRLWQLMGPAGRDNVAPRSRIGPWGF

SEQ ID NO: 99 - protein, 41 aa, synthetic construct
SFGCRFGTCTVQKLAHRLWQLMGPAGRDNVAPRWKIGPWGF

SEQ ID NO: 100 - protein, 41 aa, synthetic construct
SFGCRFGTCTVQKLAHRLWQLMGPAGRDNVAPRWRISPWGF

SEQ ID NO: 101 - protein, 41 aa, synthetic construct; identical to SEQ ID NO: 100

SEQ ID NO: 102 - protein, 41 aa, synthetic construct
SFGCRFGTCTVQKLAHRLWQLMGPAGRDNVAPRWLIGPWGF

SEQ ID NO: 103 - protein, 41 aa, synthetic construct
SFGCRFGTCTVQKLAHRLWQLMGPAGRDNVAPRWRIGPWGF

SEQ ID NO: 104 - protein, 40 aa, synthetic construct
VGCVLGTCQVQNLSHRLWQLMGPAGRDNVAPRWKISPWGY

SEQ ID NO: 105 - protein, 40 aa, synthetic construct
VGCVLGTCQVQNLSHRLWQLMGPAGRDNVAPRWKIGPWGY

SEQ ID NO: 106 - protein, 40 aa, synthetic construct
VGCVLGTCQVQNLSHRLWQLMGPAGRDNVAPRWLISPWGY

SEQ ID NO: 107 - protein, 40 aa, synthetic construct
VGCVLGTCQVQNLSHRLWQLMGPAGRDNVAPRWRISPWGY

SEQ ID NO: 108 - protein, 40 aa, synthetic construct
VGCVLGTCQVQNLSHRLWQLMGPAGRDNVAPRSLIGPWGY

SEQ ID NO: 109 - protein, 40 aa, synthetic construct
VGCVLGTCQVQNLSHRLWQLMGPAGRDNVAPRSRIGPWGY

SEQ ID NO: 110 - protein, 40 aa, synthetic construct
VGCVLGTCQVQNLSHRLWQLMGPAGRDNVAPRWKISPWGF

SEQ ID NO: 111 - protein, 40 aa, synthetic construct
VGCVLGTCQVQNLSHRLWQLMGPAGRDNVAPRSKIGPWGF

SEQ ID NO: 112 - protein, 40 aa, synthetic construct
VGCVLGTCQVQNLSHRLWQLMGPAGRDNVAPRWKIGPWGF

SEQ ID NO: 113 - protein, 40 aa, synthetic construct
VGCVLGTCQVQNLSHRLWQLMGPAGRDNVAPRWLIGPWGY

SEQ ID NO: 114 - protein, 40 aa, synthetic construct
VGCVLGTCQVQNLSHRLWQLMGPAGRDNVAPRWRIGPWGY

SEQ ID NO: 115 - protein, 40 aa, synthetic construct
VGCVLGTCQVQNLSHRLWQLMGPAGRDNVAPRSLIGPWGF

SEQ ID NO: 116 - protein, 40 aa, synthetic construct
VGCVLGTCQVQNLSHRLWQLMGPAGRDNVAPRSRIGPWGF

SEQ ID NO: 117 - protein, 40 aa, synthetic construct
VGCVLGTCQVQNLSHRLWQLMGPAGRDNVAPRWRISPWGF

SEQ ID NO: 118 - protein, 40 aa, synthetic construct; identical to SEQ ID NO: 117

SEQ ID NO: 119 - protein, 40 aa, synthetic construct
VGCVLGTCQVQNLSHRLWQLMGPAGRDNVAPRWLIGPWGF

(19) United States
(12) Patent Application Publication
(10) Pub. No.: US 2026/0209303 A1
(43) Pub. Date: Jul. 23, 2026

(54) Variants of Adrenomedullin (AM) and Adrenomedullin 2 (AM2) and Methods of Use

(71) Applicant: The Board of Regents of the University of Oklahoma, Norman, OK (US)

(72) Inventors: Augen A. PIOSZAK, Edmond, OK (US); Katie Marie BABIN, Oklahoma City, OK (US)

(21) Appl. No.: 19/571,031

(22) Filed: Mar. 18, 2026

Related U.S. Application Data
(63) Continuation-in-part of application No. 19/481,729, filed on May 6, 2024.
(60) Provisional application No. 63/773,563, filed on Mar. 18, 2025.
(60) Provisional application No. 63/910,353, filed on Nov. 3, 2025.
(60) Provisional application No. 63/920,592, filed on Nov. 19, 2025.

(51) Int. Cl. C07K 14/575; A61K 38/00

(52) U.S. Cl. CPC C07K 14/575; A61K 38/00

(57) ABSTRACT

Variant peptides of adrenomedullin (AM) and adrenomedullin 2/Intermedin (AM2/IMD) are disclosed, wherein the variant peptides have high binding affinity and agonistic or antagonistic activity for at least one receptor complex CLR:RAMP3. The AM and AM2 variants include chimeras which comprise portions of at least one of AM, AM2, CGRP, AMY, and CT. Also disclosed are methods of using the variant peptides in therapeutic treatments.

### CROSS REFERENCE TO RELATED APPLICATIONS

The present application is a continuation-in-part of U.S. patent application Ser. No. 19/481,729, filed Nov. 5, 2025; which is a U.S. national stage application filed under 35 USC § 371 of PCT Application Ser. No. PCT/US2024/027970, filed May 6, 2024; which claims priority to U.S. Provisional Application Ser. No. 63/500,316, filed May 5, 2023. The present application also claims benefit under 35 USC § 119(e) and PCT Rule 80.5 of U.S. Provisional Application Ser. No. 63/773,563, filed Mar. 18, 2025, 63/910,353, filed Nov. 3, 2025; and 63/920,592, filed Nov. 19, 2025. Each of the above-referenced patent applications is hereby expressly incorporated herein by reference in its entirety.

### STATEMENT REGARDING FEDERALLY SPONSORED RESEARCH OR DEVELOPMENT

This invention was made with government support under grant number R01GM104251 awarded by the National Institutes of Health. The government has certain rights in the invention.

### REFERENCE TO SEQUENCE LISTING SUBMITTED ELECTRONICALLY

The instant application contains, as a separate part of the present disclosure, a Sequence Listing which has been submitted via EFS-Web in computer readable form as an XML file. The Sequence Listing, created Mar. 18, 2026, is named “5864182_SequenceListing.xml” and is 127,814 bytes in size. The entire contents of the Sequence Listing are hereby incorporated herein by reference.

### BACKGROUND

The peptide Adrenomedullin 2 (AM2), also known as Intermedin (IMD), was independently discovered by two groups in 2004 based on its sequence similarity to the calcitonin/calcitonin gene-related peptide (CGRP) family of peptides, which also includes adrenomedullin (AM) and amylin (AMY). AM2 has overlapping and distinct functions, but it has remained the least understood of these peptides. Like CGRP and AM, when administered peripherally AM2 induces vasodilation, and it has protective effects in the cardiovascular, pulmonary, and renal systems. AM2 stabilizes the endothelial barrier and has anti-inflammatory actions, and these effects protected against sepsis in mice. AM2 is angiogenic and its knock-out in mice revealed roles in stimulating endothelial cell proliferation to enlarge the vascular lumen and in promoting vessel fusion. Beyond the circulatory system, AM2 protected against obesity and insulin resistance in mice by promoting beige cell biogenesis and reducing adipose inflammation, and its central administration activated the hypothalamic-pituitary-adrenal axis and increased sympathetic activity. There is considerable promise in targeting AM2 signaling for therapeutics for cardiovascular, metabolic, and other disorders, but progress has been hindered by the limited understanding of the molecular mechanisms that distinguish AM2 signaling from that of AM and CGRP.

AM, AM2, and CGRP share three heterodimeric cell surface receptors that are comprised of a common class B G protein-coupled receptor (GPCR) subunit called calcitonin receptor-like receptor (CLR, or CRLR), and one of three variable receptor activity modifying protein subunits, known as RAMP1, RAMP2, and RAMP3, that alters CLR ligand selectivity. These couple most efficiently to the stimulatory Gs protein to activate cAMP signaling, but they also couple to Gq and Gi proteins. Much of the understanding of the molecular pharmacology of the three peptides and receptors comes from studies of cAMP signaling.

CGRP is most potent at the CLR-RAMP1 complex, which is thus designated as the CGRP receptor. This receptor mediates CGRP actions in the trigeminal system and is the target of several recently approved inhibitor drugs for migraine headache. AM is most active and equally potent at the CLR-RAMP2 and CLR-RAMP3 complexes, which are termed the AM₁ and AM₂ receptors (AM₁R and AM₂R), respectively. AM₁R mediates the essential developmental actions of AM in the cardiovascular and lymphatic systems.

AM2 has been considered to be a relatively non-selective agonist of the three CLR-RAMP complexes. It exhibits a slight preference for the AM₂R, but the AM₂ nomenclature is not meant to imply that it is the AM2 receptor. In cAMP assays, AM2 is slightly less potent than CGRP and AM at the CGRP and AM₁ receptors, respectively, and is equipotent to AM at the AM₂R. Different effects of AM2 have been ascribed to signaling through either the CGRP or the AM receptors, but in many cases, the receptor(s) mediating a given AM2 action is unclear, due in part to a lack of suitable subtype-selective antagonists. There are examples of apparent mismatch between AM2 pharmacology at the cloned receptors and in vivo, but the bases for these discrepancies are unknown. Recently, the idea that AM2, AM, and CGRP promote distinct receptor-transducer coupling profiles (agonist bias) has been explored. There are some data supporting this, but other studies found less evidence for ligand bias, so the extent to which signaling bias affects the biology of the peptides has remained unclear.

Structural studies advanced the understanding of how the peptides bind the receptors and how the RAMPs modulate binding. In crystal structures of CLR-RAMP1/2-peptide ECD complexes, the C-terminal fragments of CGRP, AM, and AM2 were found to occupy a shared binding site on the CLR ECD, but with distinct, mostly unstructured conformations. RAMP1/2 augmented the ECD binding site with unique contacts to the peptides. These contributed to, but did not fully explain, ligand selectivity. Cryo-EM structures of active-state CGRP-CGRPR, AM-AM₁R, AM-AM₂R, and AM2-AM₂R complexes with Gs showed that the N-terminal half of each peptide adopted a disulfide loop and α-helix structure that similarly occupied the CLR TMD. The RAMPs did not contact the peptides beyond the ECD complexes. 3D variability analyses of the cryo-EM data were consistent with differential RAMP1-3 modulation of CLR ECD-TMD inter-domain dynamics playing a role in receptor phenotype. Despite this progress, several issues remain unresolved. How the two AM peptides bind the CLR-RAMP3 ECD complex is unclear because their C-terminal fragments in the AM₂R cryo-EM structures were modeled with different conformations than in the crystal structures. The relative contributions of RAMP augmentation of the CLR ECD peptide binding site and modulation of CLR dynamics to receptor phenotype is unclear. In addition, it has been unknown if the peptide agonists differentially modulate CLR conformation and dynamics and/or work in concert with the RAMPs to shape the signaling outcomes. The temporal features of AM2, AM, and CGRP signaling have received little attention. It is to addressing this deficiency that the presently disclosed work was directed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Aspects of several embodiments of the present disclosure are illustrated in the appended drawings. The appended drawings only illustrate several embodiments and are not intended to be limiting of the scope of the inventive concepts disclosed herein.

FIG. 1A shows CGRP, AM, and AM2 cAMP Signaling Kinetics for COS-7 cells expressing the indicated receptor and the biosensor were stimulated with 100 nM of CGRP (green circle), AM (blue square), or AM2 (orange triangle) for 15 min. followed by 10 μM antagonist challenge. (A) CLR-RAMP1 with αCGRP(8-37) [N31D/S34P/K35W/A36S] as the antagonist. CGRP with buffer addition is shown in dark green. (B) CLR-RAMP2 with AM(22-52) [S48G/Q50W] as the antagonist. AM with buffer addition is shown as dark blue. (C) CLR-RAMP3 with AM(22-52) [S48G/Q50W] as the antagonist. AM2 with buffer addition is shown as brown. Plots show a representative of three independent experiments each conducted with duplicate technical replicates. Error bars show standard deviation of technical replicates. (D) Scatter plots summarizing the decay half-life for each receptor-peptide combination as mean±SEM from three independent replicates. Star indicates significance compared to all other combinations determined by one-way ANOVA with Tukey's post hoc test.

FIG. 1B shows CGRP, AM, and AM2 cAMP Signaling Kinetics for HEK293 cells expressing the indicated receptor and the biosensor were stimulated with 100 nM of CGRP (green circle), AM (blue square), or AM2 (orange triangle) for 15 min. followed by 10 μM antagonist challenge. (A) CLR-RAMP1 with αCGRP(8-37) [N31D/S34P/K35W/A36S] as the antagonist. CGRP with buffer addition is shown in dark green. (B) CLR-RAMP2 with AM(22-52) [S48G/Q50W] as the antagonist. AM with buffer addition is shown as dark blue. (C) CLR-RAMP3 with AM(22-52) [S48G/Q50W] as the antagonist. AM2 with buffer addition is shown as brown. Plots show a representative of three independent experiments each conducted with duplicate technical replicates. Error bars show standard deviation of technical replicates. (D) Scatter plots summarizing the decay half-life for each receptor-peptide combination as mean±SEM from three independent replicates. Star indicates significance compared to all other combinations determined by one-way ANOVA with Tukey's post hoc test.

FIG. 2A shows NanoBRET™ binding kinetics of AM-TAMRA and AM2-TAMRA at NLuc-CLR:RAMP3 in membranes. (A) Cartoon depicting the position of NLuc donor and TAMRA acceptor (star) at position 35 of AM2. (B) Equilibrium binding for the indicated peptides in the absence or presence of 10 μM antagonist AM(22-52) [S48G/Q50W]. All plots show a representative of three independent experiments with duplicate technical replicates. Error bars show standard deviation for technical replicates.

FIG. 2B shows association kinetics of the experiment of FIG. 2A. (Upper panel) AM-TAMRA, Lower panel) AM2-TAMRA. All plots show a representative of three independent experiments with duplicate technical replicates. Error bars show standard deviation for technical replicates.

FIG. 2C shows (A) observed rate vs. probe concentration plot of the experiments of FIGS. 2A-2B for the indicated probes determined using the first 10 min of association data. (B) Dissociation kinetics for 10 nM AM-TAMRA or AM2-TAMRA with dissociation initiated by 1 μM AM(22-52) [S48G/Q50W]. Curves were fit to a two-phase exponential decay (solid line) and a one-phase exponential decay (dashed lines). All plots show a representative of three independent experiments with duplicate technical replicates. Error bars show standard deviation for technical replicates.

FIG. 3 shows stability of CLR-RAMP3 complexes analyzed by native PAGE. (A) Cartoon depicting detergent-solubilized heterodimer and quaternary complexes. (B and C) Membranes expressing MBP-CLR-EGFP and MBP-RAMP3 were incubated with 200 nM agonist and 50 μM miniGs to form solubilized quaternary complexes followed by challenge with 3-fold serial dilutions of AM(22-52) [S48G/Q50W] antagonist for the indicated times. (D) Heterodimer thermostability assay. The membranes were incubated at the indicated temperatures in the absence or presence of the indicated peptides (1 μM) followed by native PAGE analysis. In all panels, the heterodimer and quaternary complexes were resolved on 8% hrCNE native gels and imaged for in-gel EGFP fluorescence. All gels shown as a representative of three independent experiments.

FIG. 4 shows amino acid sequence alignment of AM(13-52) (SEQ ID NO: 2) and AM2(8-47) (SEQ ID NO: 7). Triangles indicate chimera junction points.

FIG. 5A shows results for real-time cAMP signaling kinetics using agonists and antagonists in COS-7 cells. (A) Structural depiction of agonists AM-AM2 half chimera and AM2-AM half chimera tested in B-D. (B) cAMP signaling kinetics for CLR-RAMP1 for chimeras of AM and AM2 after challenge with CGRP(8-37) [N31D/S34P/K35W/A36S] as antagonist. (C) cAMP signaling kinetics for CLR-RAMP2 for chimeras of AM and AM2 after challenge with AM(22-52) [S48G/Q50W] as antagonist. (D) cAMP signaling kinetics for CLR-RAMP3 for chimeras of AM and AM2 after challenge with AM(22-52) [S48G/Q50W] as antagonist. COS-7 cells were stimulated with 100 nM of the indicated agonist, followed by 10 μM of antagonist. In all plots, wild-type AM and AM2 control agonists are blue and orange, respectively, and the chimeras are colored according to the legend in (A). All plots are shown as mean±SD for technical replicates for a single representative of three independent experiments.

FIG. 5B shows results for real-time cAMP signaling kinetics using agonists and antagonists in COS-7 cells. (A) Structural depiction of agonists AM-AM2 ECD chimera and AM2-AM ECD chimera tested in B-D. (B) cAMP signaling kinetics for CLR-RAMP1 for chimeras of AM and AM2 ECD after challenge with CGRP(8-37) [N31D/S34P/K35W/A36S] as antagonist. (C) cAMP signaling kinetics for CLR-RAMP2 for chimeras of AM and AM2 ECD after challenge with AM(22-52) [S48G/Q50W] as antagonist. (D) cAMP signaling kinetics for CLR-RAMP3 for chimeras of AM and AM2 ECD after challenge with AM(22-52) [S48G/Q50W] as antagonist. COS-7 cells were stimulated with 100 nM of the indicated agonist, followed by 10 μM of antagonist. In all plots, wild-type AM and AM2 control agonists are blue and orange, respectively, and the chimeras are colored according to the legend in (A). All plots are shown as mean±SD for technical replicates for a single representative of three independent experiments.

FIG. 5C shows results for real-time cAMP signaling kinetics using agonists and antagonists in COS-7 cells. (A) Structural depiction of agonists AM2-AM-AM2 chimera and AM-AM2-AM chimera tested in B-D. (B) cAMP signaling kinetics for CLR-RAMP1 for chimeras of AM2-AM-AM2 and AM-AM2-AM after challenge with CGRP(8-37) [N31D/S34P/K35W/A36S] as antagonist. (C) cAMP signaling kinetics for CLR-RAMP2 for chimeras of AM2-AM-AM2 and AM-AM2-AM after challenge with AM(22-52) [S48G/Q50W] as antagonist. (D) cAMP signaling kinetics for CLR-RAMP3 for chimeras of AM2-AM-AM2 and AM-AM2-AM after challenge with AM(22-52) [S48G/Q50W] as antagonist. COS-7 cells were stimulated with 100 nM of the indicated agonist, followed by 10 μM of antagonist. In all plots, wild-type AM and AM2 control agonists are blue and orange, respectively, and the chimeras are colored according to the legend in (A). All plots are shown as mean±SD for technical replicates for a single representative of three independent experiments.

FIG. 5D shows scatter plots summarizing the decay half-lives of all the chimeric peptides compared to WT AM and WT AM2 at CLR-RAMP1 (A), CLR-RAMP2 (B), and CLR-RAMP3 (C). Statistical significance was determined by one-way ANOVA with Tukey's Post Hoc test.

FIG. 6 shows an amino acid sequence alignment of portions of RAMP2 (SEQ ID NO:54) and RAMP3 (SEQ ID NO:55) showing the chimera junction points and various regions of the sequences. Stars indicate the residues mutated in FIG. 7D, panels A and B.

FIG. 7A shows cAMP signaling kinetics results for certain RAMP2/3 chimeras. (A) shows a structural depiction of RAMP2 with RAMP3 ECD (R2wR3ECD) and RAMP3 with RAMP2 ECD (R3wR2ECD) chimeras with wild-type RAMP2 shown in yellow and RAMP3 shown in dark red. (B) cAMP signaling kinetics in COS-7 cells for the indicated wild-type CLR-RAMP2/3 or CLR-RAMP2/3 chimeras stimulated with 100 nM AM followed by 10 μM challenge with AM(22-52) [S48G/Q50W] antagonist. (C) cAMP signaling kinetics in COS-7 cells for the indicated wild-type CLR-RAMP2/3 or CLR-RAMP2/3 chimeras stimulated with 100 nM AM2 followed by 10 μM challenge with AM(22-52) [S48G/Q50W] antagonist. (D) Scatter plots summarizing decay half-lives for the receptors used in (B) and (C). WT RAMP2 is shown in light grey and RAMP3 in dark grey. RAMP2/3 chimeras are colored based on the legend in panel (A). WT AM is shown in blue and WT AM2 is shown in orange.

FIG. 7B shows cAMP signaling kinetics results for certain RAMP2/3 chimeras. (A) shows a structural depiction of RAMP2 with RAMP3 TMD (R2wR3TMD) and RAMP3 with RAMP2 TMD (R3wR2TMD) chimeras with wild-type RAMP2 shown in yellow and RAMP3 shown in dark red. (B) cAMP signaling kinetics in COS-7 cells for the indicated wild-type CLR-RAMP2/3 or CLR-RAMP2/3 chimeras stimulated with 100 nM AM followed by 10 μM challenge with AM(22-52) [S48G/Q50W] antagonist. (C) cAMP signaling kinetics in COS-7 cells for the indicated wild-type CLR-RAMP2/3 or CLR-RAMP2/3 chimeras stimulated with 100 nM AM2 followed by 10 μM challenge with AM(22-52) [S48G/Q50W] antagonist. (D) Scatter plots summarizing decay half-lives for the receptors used in (B) and (C). WT RAMP2 is shown in light grey and RAMP3 in dark grey. RAMP2/3 chimeras are colored based on the legend in panel (A). WT AM is shown in blue and WT AM2 is shown in orange.

FIG. 7C shows cAMP signaling kinetics results for certain RAMP2/3 chimeras. (A) shows a structural depiction of RAMP2 with RAMP3 C-tail (R2wR3C-tail) and RAMP3 with RAMP2 C-tail (R3wR2C-tail) chimeras with wild-type RAMP2 shown in yellow and RAMP3 shown in dark red. (B) cAMP signaling kinetics in COS-7 cells for the indicated wild-type CLR-RAMP2/3 or CLR-RAMP2/3 chimeras stimulated with 100 nM AM followed by 10 μM challenge with AM(22-52) [S48G/Q50W] antagonist. (C) cAMP signaling kinetics in COS-7 cells for the indicated wild-type CLR-RAMP2/3 or CLR-RAMP2/3 chimeras stimulated with 100 nM AM2 followed by 10 μM challenge with AM(22-52) [S48G/Q50W] antagonist. (D) Scatter plots summarizing decay half-lives for the receptors used in (B) and (C). WT RAMP2 is shown in light grey and RAMP3 in dark grey. RAMP2/3 chimeras are colored based on the legend in panel (A). WT AM is shown in blue and WT AM2 is shown in orange.

FIG. 7D shows cAMP signaling kinetics for CLR-RAMP3 wild-type and Y8G/W84F stimulated with AM, AM2, or AM-AM2-AM chimera agonist followed by antagonist challenge (A). A scatter plot summarizing decay half-life for the indicated receptor-peptide combinations in (A) is shown in (B). AM-AM2-AM half-life values at WT RAMP3 were re-plotted from FIG. 5D(C) for comparison purposes. All plots show a single representative of three independent experiments with standard deviation for duplicate technical replicates. Statistical analysis was determined with one-way ANOVA using Tukey's Post Hoc test.

FIG. 8A shows an experimental design of cAMP signaling kinetics experiments with antagonist preincubation followed by agonist co-incubation as used to produce the results shown in FIGS. 8B-8N. Truncated AM and AM2 peptide variants and chimeras were screened for antagonistic activity of cAMP signaling at each of the three CLR-RAMP complexes transiently expressed in COS-7 cells. CGRP agonist was used for CLR-RAMP1, and agonist was used for CLR-RAMP2 and CLR-RAMP3. Preincubation with 1 μM antagonist was followed by co-incubation with 10 nM agonist. Agonist-only control with no antagonist is shown in grey. X-axis is time; Y-axis is BRET ratio 475/535.

FIG. 8B shows results using the experimental procedures of FIG. 8A where the antagonist is Ac-AM2(17-47)NH₂ (SEQ ID NO:8).

FIG. 8C shows results using the experimental procedures of FIG. 8A where the antagonist is Ac-AM2(17-47)NH₂ [H45W] (SEQ ID NO: 20).

FIG. 8D shows results using the experimental procedures of FIG. 8A where the antagonist is Ac-AM2(17-33)-AM(39-52)NH₂ (SEQ ID NO:22).

FIG. 8E shows results using the experimental procedures of FIG. 8A where the antagonist is Ac-AM2(17-33)-AM(39-52)NH₂ [S45W/Q50W] (SEQ ID NO:23).

FIG. 8F shows results using the experimental procedures of FIG. 8A where the antagonist is Ac-AM2(17-33)-AM(39-52)NH₂ [S45W/Q50W/Y52F] (SEQ ID NO:24).

FIG. 8G shows results using the experimental procedures of FIG. 8A where the antagonist is Ac-AM2(17-33)-AM(39-52)NH₂ [S45W/K46L/Q50W/Y52F] (SEQ ID NO:25).

FIG. 8H shows results using the experimental procedures of FIG. 8A where the antagonist is AM(22-28)-AM2(23-33)-AM(39-52)NH₂(SEQ ID NO:26).

FIG. 8I shows results using the experimental procedures of FIG. 8A where the antagonist is AM(22-28)-AM2(23-33)-AM(39-52)NH₂ [S45W/Q50W] (SEQ ID NO:28).

FIG. 8J shows results using the experimental procedures of FIG. 8A where the antagonist is AM(22-28)-AM2(23-33)-AM(39-52)NH₂ [S45W/Q50W/Y52F] (SEQ ID NO:29).

FIG. 8K shows results using the experimental procedures of FIG. 8A where the antagonist is AM(22-28)-AM2(23-33)-AM(39-52)NH₂ [S45W/K46L/Q50W/F52Y] (SEQ ID NO:30).

FIG. 8L shows results using the experimental procedures of FIG. 8A where the antagonist is Ac-AM2(17-33)-AM(39-52)NH₂ [S45W/K46R/Q50W/Y52F] (SEQ ID NO:69).

FIG. 8M shows results using the experimental procedures of FIG. 8A where the antagonist is Ac-AM2(17-33)-AM(39-52)NH₂ [S45W/K46R/S48G/Q50W/Y52F] (SEQ ID NO:71).

FIG. 8N shows results using the experimental procedures of FIG. 8A where the antagonist is AM(22-28)-AM2(23-33)-AM(39-52)NH₂[S45W/K46R/Q50W/Y52F] (SEQ ID NO:84).

FIG. 9A shows an experimental design of cAMP signaling kinetics experiments with antagonist preincubation and washout followed by agonist addition as used to produce the results shown in FIGS. 9B-9N. Truncated AM and AM2 peptide variants and chimeras were screened for antagonistic activity of cAMP signaling at each of the three CLR-RAMP complexes transiently expressed in COS-7 cells. CGRP agonist was used for CLR-RAMP1, and AM(13-52) agonist was used for CLR-RAMP2 and CLR-RAMP3. Preincubation with 1 μM antagonist was followed by washout of antagonist and addition of 10 nM agonist. Agonist-only control with no antagonist is shown in grey. X-axis is time; Y-axis is BRET ratio 475/535.

FIG. 9B shows results using the experimental procedures of FIG. 9A where the antagonist is Ac-AM2(17-47)NH₂ (SEQ ID NO:8).

FIG. 9C shows results using the experimental procedures of FIG. 9A where the antagonist is Ac-AM2(17-47)NH₂ [H45W] (SEQ ID NO: 20).

FIG. 9D shows results using the experimental procedures of FIG. 9A where the antagonist is Ac-AM2(17-33)-AM(39-52)NH₂ (SEQ ID NO:22).

FIG. 9E shows results using the experimental procedures of FIG. 9A where the antagonist is Ac-AM2(17-33)-AM(39-52)NH₂ [S45W/Q50W] (SEQ ID NO:23).

FIG. 9F shows results using the experimental procedures of FIG. 9A where the antagonist is Ac-AM2(17-33)-AM(39-52)NH₂ [S45W/Q50W/Y52F] (SEQ ID NO:24).

FIG. 9G shows results using the experimental procedures of FIG. 9A where the antagonist is Ac-AM2(17-33)-AM(39-52)NH₂ [S45W/K46L/Q50W/Y52F] (SEQ ID NO:25).

FIG. 9H shows results using the experimental procedures of FIG. 9A where the antagonist is AM(22-28)-AM2(23-33)-AM(39-52)NH₂ (SEQ ID NO:26).

FIG. 9I shows results using the experimental procedures of FIG. 9A where the antagonist is AM(22-28)-AM2(23-33)-AM(39-52)NH₂ [S45W/Q50W] (SEQ ID NO:28).

FIG. 9J shows results using the experimental procedures of FIG. 9A where the antagonist is AM(22-28)-AM2(23-33)-AM(39-52)NH₂ [S45W/Q50W/Y52F] (SEQ ID NO:29).

FIG. 9K shows results using the experimental procedures of FIG. 9A where the antagonist is AM(22-28)-AM2(23-33)-AM(39-52)NH₂ [S45W/K46L/Q50W/F52Y] (SEQ ID NO:30).

FIG. 9L shows results using the experimental procedures of FIG. 9A where the antagonist is Ac-AM2(17-33)-AM(39-52)NH₂ [S45W/K46R/Q50W/Y52F] (SEQ ID NO:69).

FIG. 9M shows results using the experimental procedures of FIG. 9A where the antagonist is Ac-AM2(17-33)-AM(39-52)NH₂ [S45W/K46R/S48G/Q50W/Y52F] (SEQ ID NO:71).

FIG. 9N shows results using the experimental procedures of FIG. 9A where the antagonist is AM(22-28)-AM2(23-33)-AM(39-52)NH₂[S45W/K46R/Q50W/Y52F] (SEQ ID NO:84).

FIG. 10 shows results of CGRP, AM, and AM2 cAMP Signaling Kinetics. (A, B) One-phase exponential decay curve fit for CLR-RAMP3 after antagonist addition in COS-7 (A) and HEK293 (B) cells. (C) cAMP kinetics for CLR-RAMP3 in COS-7 cells at 37° C. with CGRP (green circle), AM (blue square), or AM2 (orange triangle) used as agonists and 10 μM AM(22-52) [S48G/Q50W] as the antagonist. The brown and cyan curves indicated AM2 stimulation followed by buffer addition or 10 μM forskolin control, respectively. (D) Endogenous response to 100 nM CGRP, AM, and AM2 in HEK293 cells compared to transiently transfected CLR-RAMP3 response to 100 nM AM and AM2.

FIG. 11A shows results of NanoBRET™ Binding kinetics of AM-TAMRA and AM2-TAMRA to NLuc-CLR:RAMP3 membranes. (A) cAMP accumulation assay for the indicated receptor-agonist combinations in COS-7 cells. (B) cAMP accumulation assay for the indicated peptides at CLR-RAM4P3 in COS-7 cells.

FIG. 11B shows results of NanoBRET™ Binding kinetics of AM-TAMRA and AM2-TAMRA to NLuc-CLR:RAMP3 membranes. (A) cAMP signaling kinetics at CLR-RAMP3 in COS-7 cells with 100 nM of the indicated agonist and 10 μM AM(22-52) [S48G/Q50W] antagonist. (B) Scatter plot summarizing the decay half-lives from panel B with mean±SEM of three independent replicates. AM-TAMRA had a half-life of 3.1±0.65 min and AM2-TAMRA had a half-life of 43±3.5 min. Statistical analysis done with one-way ANOVA and Tukey's post hoc test.

FIG. 11C shows results of NanoBRET™ Binding kinetics of AM-TAMRA and AM2-TAMRA to NLuc-CLR:RAMP3 membranes. (A) Equilibrium binding with 1 hour incubations time. (B) Equilibrium binding with 3 hour incubations time. (C) Equilibrium binding with 4.5 hour incubation time.

FIG. 11D shows results of NanoBRET™ Binding kinetics of AM-TAMRA and AM2-TAMRA to NLuc-CLR:RAMP3 membranes. (A,B) Association kinetics of AM-TAMRA corresponding to FIG. 2B (lower panel) with extended time (A) and AM2-TAMRA corresponding to FIG. 2C(A). (C) Raw dissociation kinetic data corresponding to FIG. 2C(B) with AM-TAMRA (blue) and AM2-TAMRA (orange) showing slight signaling decay over time.

FIG. 12 shows results of cAMP accumulation assay for AM and AM2 with CLR-RAMP3 wild-type and CLR-RAMP3 [Y83G/W84F]. CLR was co-expressed with WT RAMP3 or RAMP3 [Y83G/W84F] and stimulated with AM or AM2 in COS-7 cells. Values plotted as mean SD of technical replicates show as a representative of two independent replicates.

The following abbreviations may be used herein:
AM: Adrenomedullin,
AMY: Amylin,
AM2: Adrenomedullin 2,
AM2: Adrenomedullin 2,
AM₁R: Adrenomedullin G protein coupled receptor, a.k.a. CLR-RAMP2,
AM₂R: ‘Adrenomedullin₂ G protein coupled receptor, a.k.a. CLR-RAMP3,
ANOVA: Analysis of variance,
αCGRP: Calcitonin gene-related peptide alpha,
βCGRP: Calcitonin gene-related peptide beta,
BPEI: Branched polyethylenimine,
BRET: Bioluminescence resonance energy transfer,
cAMP: cyclic adenosine monophosphate,
CGRP: calcitonin gene-related peptide,
CLR: Calcitonin receptor-like receptor,
CLR-RAMP1: CGRP receptor,
CLR-RAMP2: AM receptor, AM₁R,
CLR-RAMP3: AM2/IMD receptor, AM₂R,
CT: Calcitonin,
CTR: Calcitonin receptor,
ECD: extracellular domain,
EGFP: Enhanced green fluorescent protein,
GPCR: G protein-coupled receptor,
IBMX: 3-isobuytl-1-methylxanthine,
IMD: Intermedin,
MD: Molecular dynamics,
MW: molecular weight,
Nluc: nanoluciferase,
PAGE: Polyacrylamide gel electrophoresis,
PEG: polyethylene glycol,
PTH: parathyroid hormone receptor,
PTHrP: parathyroid hormone related protein,
PTH1R: parathyroid hormone receptor,
RAMP1: Receptor activity modifying protein 1,
RAMP2: Receptor activity modifying protein 2,
RAMP3: Receptor activity modifying protein 3,
RMSD: root mean squared distance,
RT: room temperature,
SEM: scanning electron microscopy,
TAMRA: Tetramethylrhodamine,
TMD transmembrane domain,
WT: wild type,
7TM: 7-transmembrane.

### DETAILED DESCRIPTION

Disclosed herein are various peptides having agonistic or antagonistic effects on the activity of the CLR:RAMP1, CLR:RAMP2, and CLR:RAMP3 receptor complexes. In certain non-limiting embodiments, the peptides comprise variants of the AM, AM2 (a.k.a., IMD), CGRP, AMY, and CT peptides, the peptides which bind to the CLR:RAMP1-3 receptor complexes. In certain non-limiting embodiments, the peptides include chimeric constructs comprising portions of AM, AM2, CGRP, AMY, and CT, and variant portions of AM, AM2, CGRP, AMY, and CT. The variant and chimeric peptides can be used to treat a number of conditions and diseases involving the CLR:RAMP1, CLR:RAMP2, and CLR:RAMP3 receptor complexes.

The variant and chimeric peptides of the present disclosure have, but are not limited to, one of at least two effects: (1) an agonistic effect on the corresponding CLR:RAMP1, CLR:RAMP2, or CLR:RAMP3 receptor complex, and (2) an antagonistic effect against the corresponding CLR:RAMP1, CLR:RAMP2, or CLR:RAMP3 receptor complex. These effects lead to particular utilities. For example, variant and chimeric peptides of the present disclosure which have agonistic effects can be used therapeutically for treatment of, for example, heart failure, acute myocardial infarction, pulmonary hypertension, pre-eclampsia, lymphedema, lymphangiectasia, and fertility (e.g., implantation during in vitro fertilization), and other conditions discussed elsewhere herein. Variant and chimeric peptides of the present disclosure which have antagonistic effects can be used therapeutically for treatment of, for example, cancers, migraine headaches, and other conditions described elsewhere herein.

The variant and chimeric peptides of the present disclosure can be used to treat a number of conditions and diseases involving the CGRP, AM, and AM2 receptor complexes CLR:RAMP1, CLR:RAMP2, and CLR:RAMP3. Particular types of cancers that can be treated in a subject by the administration of the peptides of the disclosure include, but are not limited to: hepatocellular carcinoma, prostate cancer, breast cancer, adrenal tumors, glioblastoma multiforme, melanoma, acute myeloid leukemia, colon cancer, colorectal cancer, renal cancer, lung cancer, osteosarcoma, cervical cancer, ovarian cancer, and pancreatic cancer. Examples of other conditions and diseases that can be treated in a subject by the administration of variant peptides of the present disclosure include, but are not limited to: sepsis, ulcerative colitis, Crohn's disease, inflammatory bowel disease, myocardial infarction, heart failure, atherosclerotic vascular disease, tissue or organ ischemia, arteriosclerosis obliterans, Buerger's disease, pulmonary hypertension, lymphedema (primary and secondary), promotion of embryo implantation during in vitro fertilization, preeclampsia, migraine headache, hyperalgesia, menopausal hot flashes, osteoarthritis, rheumatoid arthritis, osteoporosis, pulmonary hypertension, atherosclerosis, myocardial ischemia, gut ischemia, liver ischemia, kidney ischemia, brain ischemia, and ischemic brain injury.

The present disclosure notably demonstrates that AM2 exhibits significantly longer duration cAMP signaling at the AM₂R than all other agonist-receptor pairings of this class. AM and AM2 bind the AM₂R with similar affinities, but AM2 has slower on- and off-rates resulting in a longer residence time. The regions responsible for the slow off-rate were mapped to the AM2 mid-region, which binds at the interface of the CLR ECD and TMD, and to the RAMP3 ECD; specifically, to residues that augment the CLR ECD peptide binding pocket. Swapping the mid-region of AM2 into AM surprisingly confers long-duration signaling at both AM receptors. The present results indicate that AM2 is kinetically selective for AM₂R, reveal the structural basis for its distinct kinetics, and show how the peptide agonist and RAMP accessory protein collaborate to dictate CLR pharmacology. These results advance our mechanistic understanding of RAMP modulation of GPCR function. Overall, we conclude that AM2 is the endogenous agonist of the AM₂R and that its long-acting signaling at this receptor is the distinguishing feature of AM2 biology.

Before further describing various embodiments of the peptides, peptide compounds, compositions, and methods of the present disclosure in more detail by way of exemplary description, examples, and results, it is to be understood that the peptides, peptide compounds, compositions, and methods of the present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning, and the embodiments and examples are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. Thus, while the peptides, peptide compounds, compositions, and methods of the present disclosure have been described in terms of particular embodiments, it will be apparent to those of skill in the art that variations may be applied to the peptides, peptide compounds, compositions, and/or methods and in the steps or in the sequence of steps of the methods described herein without departing from the concept, spirit, and scope of the inventive concepts.

All patents, published patent applications, and non-patent publications mentioned in the specification or referenced in any portion of this application, including U.S. Provisional Patent Applications Ser. Nos. 63/773,563 (filed Mar. 18, 2025), 63/910,353 (filed Nov. 3, 2025), 63/920,592 (filed Nov. 19, 2025), and 63/500,316 (filed May 5, 2023), PCT Application No. PCT/US2024/027970 (filed May 6, 2024), and U.S. patent application Ser. No. 19/481,729 filed Nov. 5, 2025, are hereby expressly incorporated herein by reference in their entirety to the same extent as if each individual patent application or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1). An amino acid sequence having a length in a range of 12 to 50 amino acids, for example, refers to a peptide or oligopeptide oligonucleotide having at least 12 amino acids and less than 51 amino acids, and includes any range bounded by two different integers in said range of 12, 13, 14, 15, 16, 17, 18, 19, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 amino acids, including for example 18 to 25, 20 to 24, or 20-22.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately,” where used herein when referring to a measurable value such as an amount, a temporal duration, and the like, is meant to encompass, for example, variations of ±25%, or ±20%, or ±15%, ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

Where used herein the term “active agent” refers to a presently disclosed or described variant or chimeric peptide, or a compound, complex, coacervate, and/or conjugate comprising such variant or chimeric peptide, which has either agonistic or antagonistic behavior toward at least one of the CLR:RAMP1-3 receptor complexes. By “biologically active” is meant the ability to modify the molecular, biochemical, or physiological system of a cell, organ, or organism, without reference to how the active agent has its physiological effects. In one non-limiting embodiment, biologically active refers to the ability of an active agent to target and either promote or interfere with the normal function of one or of the CLR:RAMP1-3 receptor complexes.

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Where used herein, the pronouns “we” or “us” or the possessive determiner “our” are intended to refer to all persons involved in a particular aspect of the investigation disclosed herein and as such may include non-inventor laboratory personnel, assistants, technicians, collaborators and/or contributors who worked under the supervision of the inventor(s), and thus are not intended to represent an inventorship role by said laboratory personnel, assistants, technicians, collaborators, and/or contributors in any subject matter disclosed herein.

Non-limiting examples of animals within the scope and meaning of this term include dogs, cats, rats, mice, guinea pigs, chinchillas, horses, goats, cattle, sheep, zoo animals, Old and New World monkeys, non-human primates, and humans.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention. Non-limiting examples of modes of administration include oral, topical, retrobulbar, subconjunctival, transdermal, parenteral, subcutaneous, intranasal, intramuscular, intraperitoneal, intravitreal, and intravenous routes, including both local and systemic applications. The term “topical” is used herein to define a mode of administration through an epithelial surface, such as but not limited to, the skin, eye, or internal epithelial surfaces. In addition, the compositions of the present disclosure may be designed to provide delayed, controlled, extended, and/or sustained release using formulation techniques which are well known in the art.

The terms “therapeutic composition” and “pharmaceutical composition” refer to a composition containing a peptide as described herein that may be administered to a subject by any method known in the art or otherwise contemplated herein, wherein administration of the composition brings about a therapeutic effect as described elsewhere herein.

The term “effective amount” refers to an amount of a peptide or peptide compound which is sufficient to exhibit a detectable therapeutic, amelioration, or treatment effect in a subject without excessive adverse side effects (such as substantial toxicity, irritation and allergic response) commensurate with a reasonable benefit/risk ratio when used in the manner of the present disclosure. The effective amount for a subject will depend upon the subject's type, size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition or a symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling, or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most or all adverse symptoms, complications, consequences or underlying causes associated with the condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition (e.g., stabilizing), over a short or long duration of time (e.g., seconds, minutes, hours).

As used herein, the phrase “biologically active” refers to a substance that has activity in a biological system (e.g., in a cell (e.g., isolated, in culture, in a tissue, in an organism), in a cell culture, in a tissue, in an organism, etc.). For instance, a substance that, when administered to an organism, has a biological effect on that organism, is considered to be biologically active. It will be appreciated by those skilled in the art that often only a portion or fragment of a biologically active substance is required (e.g., is necessary and sufficient) for the activity to be present; in such circumstances, that portion or fragment is considered to be a “biologically active” portion or fragment.

As used herein, the term “small molecule” means a low molecular weight organic compound that may serve as an enzyme substrate or regulator of biological processes. In general, a “small molecule” is a molecule that is less than about 5 kilodaltons (kD) in size. In some non-limiting embodiments, provided nanoparticles further include one or more small molecules. In some non-limiting embodiments, the small molecule is less than about 4 kD, 3 kD, about 2 kD, or about 1 kD. In some non-limiting embodiments, the small molecule is less than about 800 daltons (D), about 600 D, about 500 D, about 400 D, about 300 D, about 200 D, or about 100 D. In some non-limiting embodiments, a small molecule is less than about 2000 g/mol, less than about 1500 g/mol, less than about 1000 g/mol, less than about 800 g/mol, or less than about 500 g/mol. In some non-limiting embodiments, one or more small molecules are encapsulated within the nanoparticle. In some non-limiting embodiments, small molecules are non-polymeric. In some non-limiting embodiments, in accordance with the present disclosure, small molecules are not proteins, polypeptides, oligopeptides, peptides, polynucleotides, oligonucleotides, polysaccharides, glycoproteins, proteoglycans, etc. In some non-limiting embodiments, a small molecule is a therapeutic. In some non-limiting embodiments, a small molecule is an adjuvant. In some non-limiting embodiments, a small molecule is a drug.

In some non-limiting embodiments, provided agents and/or compositions comprising such agents may be provided in particles. Particles as used in this context means nanoparticles or microparticles (or in some instances larger particles) which can consist in whole or in part of provided agent(s) and/or other therapeutic agent(s) as described herein. Such particles may contain the agent(s) and/or compositions in a core surrounded by a coating, including, but not limited to, an enteric coating. The agent(s) and/or compositions also may be dispersed throughout the particles. The agent(s) and/or compositions also maybe adsorbed into the particles. The particles maybe of any order release kinetics, including zero-order release, first-order release, second-order release, delayed release, sustained release, immediate release, and any combination thereof, etc. The particle may include, in addition to the agent(s) and/or compositions, any of those materials routinely used in the art of pharmacy and medicine, including, but not limited to, erodible, nonerodible, biodegradable, or nonbiodegradable material or combinations thereof. The particles maybe microcapsules which comprise one or more provided agents in a solution or in a semi-solid state. The particles may be of virtually any shape.

According to various embodiments, both non-biodegradable and biodegradable polymeric materials can be used in the manufacture of particles for delivering provided agent(s) and/or compositions. Such polymers maybe natural or synthetic polymers. In many embodiments, a polymer is selected based on the period of time over which release is desired. Bioadhesive polymers of particular interest include bioerodible hydrogels which may comprise, for example, polyhyaluronic acids, casein, gelatin, glutin, polyanhydrides, polyacrylic acid, alginate, chitosan, poly(methylmethacrylates), poly(ethylmethacrylates), poly(butylmethacrylate), poly(isobutylmethacrylate), poly(hexylmethacrylate), poly(isodecylmethacrylate), poly(laurylmethacrylate), poly(phenylmethacrylate), poly(methylacrylate), poly(isopropylacrylate), poly(isobutylacrylate), and poly(octadecylacrylate). In some non-limiting embodiments, provided agents and/or compositions comprising such agents maybe contained in controlled release systems.

The term “controlled release” in this context is intended to refer to any drug-containing formulation in which the manner and profile of drug release from the formulation are controlled. This refers to immediate as well as non-immediate release formulations, with non-immediate release formulations including but not limited to sustained release and delayed release formulations.

The term “sustained release” (also referred to as “extended release”) is used in this context in its conventional sense to refer to a drug formulation that provides for gradual release of a drug over an extended period of time, and that in certain particular (but non-limiting) embodiments, although not necessarily, results in substantially constant blood levels of a drug over an extended time period. The term “delayed release” is used in this context its conventional sense to refer to a drug formulation in which there is a time delay between administration of the formulation and the release of the drug there from. “Delayed release” may or may not involve gradual release of drug over an extended period of time, and thus may or may not be “sustained release.” In some non-limiting embodiments, use of a long-term sustained release implant maybe particularly suitable for treatment of chronic conditions with one or more provided agents. “Long-term” release, as used in this context, means that an implant is constructed and arranged to deliver therapeutic levels of the active ingredient for at least 7 days, and in certain non-limiting embodiments, 30-60 days. Long-term sustained release implants are well-known to those of ordinary skill in the art and include some of the release systems described elsewhere herein.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function similarly to a naturally occurring amino acid. The terms “amino acid” and “amino acid residue” are used interchangeably throughout.

The term “mutant” or “variant” is intended to refer to a protein, peptide, nucleic acid or organism which has at least one amino acid or nucleotide which is different from the wild type version of the protein, peptide, nucleic acid, or organism and includes, but is not limited to, point substitutions, multiple contiguous or non-contiguous substitutions, chimeras, or fusion proteins, and the nucleic acids which encode them.

The terms “chimera” and “chimeric” when used in regard to a peptide are intended to refer to unnatural constructs comprising bioactive amino acid sequences from at least two different peptides. Non-limiting examples of chimeras include combinations of portions of AM and AM2, AM and CGRP, AM and CT, AM and AMY, AM2 and CGRP, AM2 and CT, AM2 and AMY, CGRP and AMY, CGRP and CT, and CT and AMY. The chimeric peptides may also include substitutions of the wild type amino acids in one or more of the portions of the combination.

Examples of conservative amino acid substitutions include, but are not limited to, substitutions made within the same group such as within the group of basic amino acids (such as arginine, lysine, histidine), acidic amino acids (such as glutamic acid and aspartic acid), polar amino acids (such as glutamine and asparagine), hydrophobic amino acids (such as leucine, isoleucine, and valine), aromatic amino acids (such as phenylalanine, tryptophan, tyrosine) and small amino acids (such as glycine, alanine, serine, threonine, methionine). Other examples of possible substitutions are described below.

The term “homologous” or “% identity” as used herein means a nucleic acid (or fragment thereof) or a protein (or a fragment thereof) having a degree of homology to the corresponding natural reference nucleic acid or protein that may be in excess of 70%, or in excess of 80%, or in excess of 85%, or in excess of 90%, or in excess of 91%, or in excess of 92%, or in excess of 93%, or in excess of 94%, or in excess of 95%, or in excess of 96%, or in excess of 97%, or in excess of 98%, or in excess of 99%. For example, in regard to peptides or polypeptides, the percentage of homology or identity as described herein is typically calculated as the percentage of amino acid residues found in the smaller of the two sequences which align with identical amino acid residues in the sequence being compared, when four gaps in a length of 100 amino acids may be introduced to assist in that alignment (as set forth by Dayhoff, in Atlas of Protein Sequence and Structure, Vol. 5, p. 124, National Biochemical Research Foundation, Washington, D.C. (1972)). In one non-limiting embodiment, the percentage homology as described above is calculated as the percentage of the components found in the smaller of the two sequences that may also be found in the larger of the two sequences (with the introduction of gaps), with a component being defined as a sequence of four, contiguous amino acids. Also included as substantially homologous is any protein product which may be isolated by virtue of cross-reactivity with antibodies to the native protein product. Sequence identity or homology can be determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul (Proc. Natl. Acad. Sci. USA (1990) 87:2264-2268), modified as in Karlin & Altschul (Proc. Natl. Acad. Sci. USA (1993) 90:5873-5877).

In one non-limiting embodiment, “% identity” represents the number of amino acids or nucleotides which are identical at corresponding positions in two sequences of a protein having the same activity or encoding similar proteins. For example, two amino acid sequences each having 100 residues will have 95% identity when 95 of the amino acids at corresponding positions are the same.

Another example of a mathematical algorithm used for comparison of sequences is the algorithm of Myers & Miller (CABIOS (1988) 4:11-17). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman (Proc. Natl. Acad. Sci. USA (1988) 85:2444-2448).

Another algorithm is the WU-BLAST (Washington University BLAST) version 2.0 software (WU-BLAST version 2.0 executable programs for several UNIX platforms). This program is based on WU-BLAST version 1.4, which in turn is based on the public domain NCBI-BLAST version 1.4 (Altschul & Gish, Methods in Enzymology (1996) 266:460-480; Altschul et al., J Molec Biol. (1990) 215:403-410; Gish & States, Nature Genetics (1993) 3:266-272; Karlin & Altschul, Proc. Natl. Acad. Sci. USA (1993) 90:5873-5877; all of which are incorporated by reference herein).

In addition to those otherwise mentioned herein, mention is made also of the programs BLAST, gapped BLAST, BLASTN, BLASTP, and PSI-BLAST, provided by the National Center for Biotechnology Information. These programs are widely used in the art for this purpose and can align homologous regions of two amino acid sequences. In all search programs in the suite, the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Specific amino acids may be referred to herein by the following designations: alanine: ala or A; arginine: arg or R; asparagine: asn or N; aspartic acid: asp or D; cysteine: cys or C; glutamic acid: glu or E; glutamine: gln or Q; glycine: gly or G; histidine: his or H; isoleucine: ile or I; leucine: leu or L; lysine: lys or K; methionine: met or M; phenylalanine: phe or F; proline: pro or P; serine: ser or S; threonine: thr or T; tryptophan: trp or W; tyrosine: tyr or Y; and valine: val or V.

The terms “polynucleotide sequence” or “nucleic acid,” as used herein, include any polynucleotide sequence which encodes a variant, chimeric, or mutant peptide including polynucleotides in the form of RNA, such as mRNA, or in the form of DNA, including, for instance, cDNA and genomic DNA obtained by cloning or produced by chemical synthetic techniques or by a combination thereof. The DNA may be double-stranded or single-stranded. Single-stranded DNA may be the coding strand, also known as the sense strand, or it may be the non-coding strand, also referred to as the anti-sense strand. The polynucleotide sequence encoding a mutant peptide or encoding a therapeutically-effective fragment of a mutant peptide can be substantially the same as the coding sequence of the endogenous coding sequence as long as it encodes a biologically active mutant peptide. Further, the mutant peptide, or therapeutically-effective fragment of a mutant peptide may be expressed using polynucleotide sequence(s) which differ in codon usage due to the degeneracies of the genetic code or allelic variations.

As noted above, the peptides of the present disclosure, and the nucleic acids which encode them, include peptide and nucleic acid variants which comprise additional conservative substitutions. For example, the variant peptides include, but are not limited to, variants that are not exactly the same as the sequences disclosed herein, but which have, in addition to the substitutions explicitly described for various sequences listed herein, conservative substitutions of amino acid residues which do substantially not impair the agonistic or antagonistic activity or properties of the variants described herein. Examples of such conservative amino acid substitutions include, but are not limited to, ala to gly, ser, or thr; arg to gln, his, or lys; asn to asp, gln, his, lys, ser, or thr; asp to asn or glu; cys to ser; gln to arg, asn, glu, his, lys, or met; glu to asp, gln, or lys; gly to pro or ala; his to arg, asn, gln, or tyr; ile to leu, met, or val; leu to ile, met, phe, or val; lys to arg, asn, gln, or glu; met to gln, ile, leu, or val; phe to leu, met, trp, or tyr; ser to ala, asn, met, or thr; thr to ala, asn, ser, or met; trp to phe or tyr; tyr to his, phe or trp; and val to ile, leu, or met.

Where referred to herein, the terms “variant” and “mutant” are used interchangeably. A variant peptide, peptide variant, or mutant refers to any peptide described herein, including fragments thereof, comprising at least one amino acid substitution in the wild type amino acid sequence or in a fragment thereof. Non-limiting examples of such variant peptides are described in further detail below. The peptides of the present disclosure may be combined with one or more pharmaceutically-acceptable excipients, including carriers, vehicles, diluents, and adjuvants which may improve solubility, deliverability, dispersion, stability, and/or conformational integrity of the compounds or conjugates thereof.

In certain non-limiting embodiments, the carrier molecule may be a protein, or a polymeric material such as polyethylene glycol (PEG). The protein of polymeric material may be linked to the peptide variant via PEGylation. For example, the peptides may be conjugated or otherwise attached to a suitable carrier molecule such as, but not limited to, keyhole limpet haemocyanin (KLH), ovalbumin, bovine serum albumin (BSA), or human serum albumin (HSA). Other examples of carrier proteins which may be used include, but are not limited to, those disclosed in US Published Patent Applications 2013/0072881, 2013/0209503, and 2013/0337006, the disclosures of which are expressly incorporated herein by reference. The peptides may be bound directly to the carrier molecule or linked via a linker compound or linker peptide, for example as described in the US Published Patent Applications 2013/0072881, 2013/0209503, and 2013/0337006.

In at least certain non-limiting embodiments of the present disclosure, the variant peptide is linked to a PEG molecule (also known as poly(ethylene oxide) and poly(oxyethylene)). PEG comprises repeating units of ethylene glycol, and is available in different average molecular weights (MW) based on the average number of ethylene glycol units in the PEG molecules of the particular PEG composition. For example, PEGss, a PEG molecule with 2 ethylene glycol units, has a MW of 88 Daltons (Da). PEG₄₀₀, a PEG molecule with about 8 ethylene glycol units, has a MW of 400 Daltons (Da). PEG_{60,000}, a PEG molecule with about 1364 ethylene glycol units, has a MW of about 60,000. The PEG molecule may comprise up to 30,000 ethylene glycol units, Other examples include, but are not limited to, PEG₂₀₀ having an average MW of about 200 Daltons (Da), PEG₃₀₀ having an average MW of about 300 Da, PEG₄₀₀ having an average MW of about 400 Da, PEG₅₀₀ having an average MW of about 500 Da, PEG₇₅₀ having an average MW of about 750 Da, PEG₁₀₀₀ having an average MW of about 1000 Da, PEG₁₅₀₀ having an average MW of about 1500 Da, PEG₂₀₀₀ having an average MW of about 2000 Da, PEG₃₀₀₀ having an average MW of about 3000 Da, PEG₃₃₅₀ having an average MW of about 3350 Da, PEG₃₅₀₀ having an average MW of about 3500 Da, PEG₄₀₀₀ having an average MW of about 4000 Da, PEG₅₀₀₀ having an average MW of about 5000 Da, PEG₆₀₀₀ having an average MW of about 6000 Da, PEG₇₅₀₀ having an average MW of about 7500 Da, PEG_{10,000} having an average MW of about 10,000 Da, PEG_{15,000} having an average MW of about 15,000 Da, PEG_{20,000} having an average MW of about 20,000 Da, PEG_{25,000} having an average MW of about 25,000 Da, PEG_{30,000} having an average MW of about 30,000 Da, PEG_{40,000} having an average MW of about 40,000 Da, PEG_{50,000} having an average MW of about 50,000 Da, and PEG_{60,000} having an average MW of about 60,000 Da. Where used herein the term PEG is intended to refer to any of the examples of PEG listed above, and to PEGs having MWs in the range of 88 and 60,000, unless a particular MW is specified.

In particular, non-limiting examples, the active agent of the present disclosure is delivered via a liposomal composition. In addition to other pharmaceutically acceptable carrier(s), the liposome may contain amphipathic agents such as lipids which exist in an aggregated form as micelles, insoluble monolayers, liquid crystals, or lamellar layers in aqueous solution. Suitable lipids for liposomal formulation include, but are not limited to, monoglycerides, diglycerides, sulfatides, lysolecithin, phospholipids, saponin, bile acids, combinations thereof, and the like. Preparation of such liposomal formulations is well within the level of ordinary skill in the art, as disclosed, for example, in U.S. Pat. Nos. 4,235,871; 4,501,728; 4,837,028; and 4,737,323; the entire contents of each of which are incorporated herein by reference. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior. The aqueous portion contains the active agent to be delivered. Liposomes can be made from phospholipids other than naturally-derived phosphatidylcholine. Neutral liposome compositions, for example, can be formed from dimyristoyl phosphatidylcholine (DMPC) or dipalmitoyl phosphatidylcholine (DPPC) or other similar lipids. Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example (but not by way of limitation), soybean PC, and egg PC. Another type is formed from mixtures of phospholipid and/or phosphatidylcholine and/or cholesterol.

“Pharmaceutically acceptable salts” means salts of active agent compounds disclosed herein which are pharmaceutically acceptable, as defined above, and which possess the desired pharmacological activity. Such salts include (but are not limited to) acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include (but are not limited to) base addition salts which may be formed when acidic protons present are capable of reacting with inorganic or organic bases. Acceptable inorganic bases include (but are not limited to) sodium hydroxide, sodium carbonate, potassium hydroxide, aluminum hydroxide and calcium hydroxide. Acceptable organic bases include (but are not limited to) ethanolamine, diethanolamine, triethanolamine, tromethamine, N-methylglucamine and the like. It should be recognized that the particular anion or cation forming a part of any salt of the present disclosure is not critical, so long as the salt, as a whole, is pharmacologically acceptable. Additional non-limiting examples of pharmaceutically acceptable salts and their methods of preparation and use are shown in Handbook of Pharmaceutical Salts: Properties, and Use (P. H. Stahl & C. G. Wermuth eds., Verlag Helvetica Chimica Acta, 2002).

The term “coadministration” refers to administration of two or more active agents, e.g., a cancer or cardiac-targeted peptide as described herein and another active agent. The timing of coadministration depends in part on the combination and compositions administered and can include administration at the same time, just prior to, or just after the administration of one or more additional therapies. “Coadministration” is meant to include simultaneous or sequential administration of the compound and/or composition individually or in combination. Thus, the preparations can also be combined, when desired, with other active substances (e.g., to reduce metabolic degradation). For example, the compositions described herein can be used in combination with one another, or with other active agents known to be useful in treating the targeted condition or disease.

The active agents of the present disclosure may be present in the pharmaceutical compositions (alone or in combination) at any concentration that allows the pharmaceutical composition to function in accordance with the present disclosure; for example, but not by way of limitation, the active agent (s) may be present in a carrier, diluent, or buffer solution in a wt/wt or vol/vol range having a lower level selected from 0.00001%, 0.0001%, 0.005%, 0.001%, 0.005%, 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9% and 2.0%; and an upper level selected from 3%, 3.5%, 4%, 4.5%, 5%, 5.5%, 6%, 6.5%, 7%, 7.5% 8%, 8.5%, 9%, 9.5% 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, and 95%. Non-limiting examples of particular wt/wt or vol/vol ranges include a range of from about 0.0001% to about 95%, a range of from about 0.001% to about 75%; a range of from about 0.005% to about 50%; a range of from about 0.01% to about 40%; a range of from about 0.05% to about 35%; a range of from about 0.1% to about 30%; a range of from about 0.1% to about 25%; a range of from about 0.1% to about 20%; a range of from about 1% to about 15%; a range of from about 2% to about 12%; a range of from about 5% to about 10%; and the like. Any other range that includes a lower level selected from the above-listed lower level concentrations and an upper level selected from the above-listed upper level concentrations also falls within the scope of the present disclosure. Percentages used herein may be weight percentages (wt %) or volume percentages (vol %).

Certain non-limiting embodiments of the present disclosure are directed to a method that comprises administering to a subject in need thereof any of the pharmaceutical compositions disclosed or otherwise contemplated herein.

The pharmaceutical compositions may be administered via any mechanisms disclosed herein or otherwise contemplatable by a person having ordinary skill in the art. In one non-limiting embodiment, the administration occurs via an inhaler, which aerosolizes the active agents.

The pharmaceutical compositions of the present disclosure may be administered for any purpose disclosed or otherwise contemplated herein, as well as for any purpose within the purview of a person having ordinary skill in the art. In one non-limiting embodiment, the pharmaceutical compositions are administered in a method of treating or reducing the occurrence of cancer. However, this treatment method is not to be construed as limiting of the present disclosure, and any diseases, disorders, or conditions disclosed herein or otherwise contemplatable by a person having ordinary skill in the art (given the subject application) which may derive a therapeutic effect by treatment with the compositions disclosed herein also fall within the scope of the methods of the present disclosure.

Practice of the methods of the present disclosure may comprise administering to a subject therapeutically effective amounts of the active agents in any suitable systemic and/or local formulation, in an amount effective to deliver the dosages listed herein. The dosage can be administered, for example but not by way of limitation, on a one-time basis, or administered at multiple times (for example but not by way of limitation, from one to five times per day, or once or twice per week), or continuously via a venous drip, depending on the desired therapeutic effect. In one non-limiting example of a therapeutic method of the present disclosure, the active agent is provided in an IV infusion in the range of from about 0.01 mg/kg to about 10 mg/kg of body weight once a day.

The compositions and dosage forms of the present disclosure can be administered in a single dose treatment or in multiple dose treatments on a schedule and over a time period appropriate to the age, weight, and condition of the subject, the particular composition used, and the route of administration. In one non-limiting embodiment, a single dose of the composition according to the disclosure is administered. In other non-limiting embodiments, multiple doses are administered. The frequency of administration can vary depending on any of a variety of factors, e.g., severity of the symptoms, or whether the composition is used for prophylactic or curative purposes. For example, in certain non-limiting embodiments, the composition is administered once per month, twice per month, three times per month, every other week, once per week, twice per week, three times per week, four times per week, five times per week, six times per week, every other day, daily, twice a day, or three times a day. The duration of treatment (i.e., the period of time over which the composition is administered) can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. Where used herein, unless otherwise indicated, the dosage amount refers to the amount of active pharmaceutical ingredient (API) that is administered to the subject. The total dose to be delivered can be provided in a single capsule, tablet, injection, or bolus (or other dosage form), or in multiple capsules, tablets, injections, or boluses (or other dosage forms).

The dosage of an administered active agent for humans will vary depending upon factors such as (but not limited to) the patient's age, weight, height, sex, general medical condition, and previous medical history. In certain non-limiting embodiments, an effective amount or therapeutic dosage of a pharmaceutical composition of the present disclosure contains, sufficient active agent to deliver from about 0.001 μg/kg to about 100 mg/kg (weight of active agent/body weight of the subject). For example, the composition will deliver about 0.01 μg/kg to about 50 mg/kg, and more particularly about 0.1 μg/kg to about 10 mg/kg, and more particularly about 1 μg/kg to about 1 mg/kg. Practice of a method of the present disclosure may comprise administering to a subject an effective amount of the active agent in any suitable systemic and/or local formulation, in an amount effective to deliver the therapeutic dosage of the active agent. In certain non-limiting embodiments, an effective dosage may be, in a range of about 1 μg/kg to about 1 mg/kg of the active agent.

In certain non-limiting embodiments, the recipient may be provided with a dosage of the active agent that is in the range of from about 1 mg to about 1000 mg, and it may be administered as a single infusion or multiple injections, although a lower or higher dosage also may be administered. In certain non-limiting embodiments, the dosage may be in the range of from about 25 mg to about 100 mg of the active agent per square meter (m²) of body surface area for a typical adult, although a lower or higher dosage also may be administered. Non-limiting examples of dosages of the active agent that may be administered to a human subject include, but are not limited to, those in ranges of 1 to 1000 mg, 1 to 600 mg, 1 to 500 mg, 1 to 400 mg, 1 to 300 mg, 1 to 200 mg, 100 to 600 mg, 100 to 500 mg, 100 to 400 mg, 100 to 300 mg, 100 to 200 mg, 150 to 600 mg, 150 to 500 mg, 150 to 400 mg, 150 to 300 mg, 150 to 250 mg, 150 to 200 mg, 200 to 7500 mg, 200 to 600 mg, 200 to 500 mg, 200 to 400 mg, 200 to 300 mg, and 200 to 250 mg, or any subrange within any of the aforementioned ranges. Dosages may be repeated as needed, for example (but not by way of limitation), once per week for 4-10 weeks, once per week for 8 weeks, or once per week for 4 weeks. It may also be given less frequently, such as (but not limited to) every other week for several months, or more frequently, such as twice weekly or by continuous infusion.

In certain non-limiting embodiments, the present disclosure is directed to a dosing regimen comprising multiple dosing cycles (e.g., wherein the first dosing cycle is a step-up, fractionated dosing cycle). In some non-limiting embodiments, the dose may range from 50 mg to 200 mg (e.g., from 50 mg to 175 mg, from 50 mg to 150 mg, from 50 mg to 125 mg, from 50 mg to 100 mg, from 50 mg to 75 mg, from 50 mg to 70 mg, from 52 mg to 100 mg, from 52 mg to 75 mg, from 50 mg to 180 mg, from 55 mg to 150 mg, from 55 mg to 100 mg, from 55 mg to 70 mg, from 55 mg to 65 mg, from 58 mg to 62 mg; e.g., about 60 mg, or any subrange within any of the aforementioned ranges).

In some non-limiting embodiments, the dose is from 20 mg to 200 mg (e.g., from 20 mg to 175 mg, from 20 mg to 150 mg, from 20 mg to 100 mg, from 20 mg to 75 mg, from 30 mg to 175 mg, from 40 mg to 175 mg, from 45 mg to 175 mg, from 50 mg to 175 mg, from 30 mg to 150 mg, from 40 mg to 100 mg, from 45 mg to 75 mg, from 50 mg to 70 mg, from 55 mg to 65 mg, from 58 mg to 62 mg; about 20 mg, about 30 mg, about 45 mg, or e.g., about 60 mg, or any subrange within any of the aforementioned ranges). In some non-limiting embodiments, the dose is from about 12 mg to about 48 mg (e.g., from about 12 mg to about 42 mg, from about 12 mg to about 36 mg, from about 12 mg to about 30 mg, from about 18 mg to about 48 mg, from about 18 mg to about 42 mg, from about 24 mg to about 42 mg, from about 27 mg to about 42 mg, from about 24 mg to about 36 mg, from about 27 mg to about 33 mg, from about 28 mg to about 32 mg; e.g., about 24 mg, about 27 mg, about 30 mg, about 33 mg, or about 36 mg, or any subrange within any of the aforementioned ranges).

In some non-limiting embodiments, the dosing regimen comprises administration of a dose in a range of from 100 mg to 750 mg (e.g., from 100 mg to 725 mg, from 100 mg to 700 mg, from 100 mg to 675 mg, from 100 mg to 650 mg, from 100 mg to 625 mg, from 100 mg to 600 mg, from 100 mg to 575 mg, from 100 mg to 550 mg, from 100 mg to 525 mg, from 100 mg to 500 mg, from 100 mg to 475 mg, from 100 mg to 450 mg, from 100 mg to 425 mg, from 100 mg to 400 mg, from 100 mg to 375 mg, from 100 mg to 350 mg, from 100 mg to 325 mg, from 100 mg to 300 mg, from 100 mg to 275 mg, from 100 mg to 250 mg, or from 100 mg to 225 mg, from 100 mg to 200 mg, from 100 mg to 175 mg, from 100 mg to 150 mg, or from 100 mg to 125 mg, or any subrange within any of the aforementioned ranges).

In some non-limiting embodiments, the dosing regimen comprises administration of a dose in a range of from 200 mg to 750 mg (e.g., from 200 mg to 725 mg, from 200 mg to 700 mg, from 200 mg to 675 mg, from 200 mg to 650 mg, from 200 mg to 625 mg, from 200 mg to 600 mg, from 200 mg to 575 mg, from 200 mg to 550 mg, from 200 mg to 525 mg, from 200 mg to 500 mg, from 200 mg to 475 mg, from 200 mg to 450 mg, from 200 mg to 425 mg, from 200 mg to 400 mg, from 200 mg to 375 mg, from 200 mg to 350 mg, from 200 mg to 325 mg, from 200 mg to 300 mg, from 200 mg to 275 mg, from 200 mg to 250 mg, or from 200 mg to 225 mg, or any subrange within any of the aforementioned ranges).

In some non-limiting embodiments, the dosing regimen comprises administration of a dose in a range of from 300 mg to 750 mg (e.g., from 300 mg to 725 mg, from 300 mg to 700 mg, from 300 mg to 675 mg, from 300 mg to 650 mg, from 300 mg to 625 mg, from 300 mg to 600 mg, from 300 mg to 575 mg, from 300 mg to 550 mg, from 300 mg to 525 mg, from 300 mg to 500 mg, from 300 mg to 475 mg, from 300 mg to 450 mg, from 300 mg to 425 mg, from 300 mg to 400 mg, from 300 mg to 375 mg, from 300 mg to 350 mg, or from 300 mg to 325 mg, or any subrange within any of the aforementioned ranges).

In some non-limiting embodiments, the dosing regimen comprises administration of a dose in a range of from 400 mg to 750 mg (e.g., from 400 mg to 725 mg, from 400 mg to 700 mg, from 400 mg to 675 mg, from 400 mg to 650 mg, from 400 mg to 625 mg, from 400 mg to 600 mg, from 400 mg to 575 mg, from 400 mg to 550 mg, from 400 mg to 525 mg, from 400 mg to 500 mg, from 400 mg to 475 mg, from 400 mg to 450 mg, or from 400 mg to 425 mg, or any subrange within any of the aforementioned ranges).

In some non-limiting embodiments, the dosing regimen comprises administration of a dose in a range of from 500 mg to 750 mg (e.g., from 500 mg to 725 mg, from 500 mg to 700 mg, from 500 mg to 675 mg, from 500 mg to 650 mg, from 500 mg to 625 mg, from 500 mg to 600 mg, from 500 mg to 575 mg, from 500 mg to 550 mg, or from 500 mg to 525 mg, or any subrange within any of the aforementioned ranges).

In some non-limiting embodiments, the dosing regimen comprises administration of a dose in a range of from 600 mg to 750 mg (e.g., from 600 mg to 725 mg, from 600 mg to 700 mg, from 600 mg to 675 mg, from 600 mg to 650 mg, from 600 mg to 625 mg, or any subrange within any of the ranges mentioned herein).

In some non-limiting embodiments, the active agent is provided in a concentration of about 1 nM, about 5 nM, about 10 nM, about 25 nM, about 50 nM, about 75 nM, about 100 nM, about 150 nM, about 200 nM, about 250 nM, about 300 nM, about 350 nM, about 400 nM, about 500 nM, about 550 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 60 μM, about 70 μM, about 75 μM, about 80 μM, about 90 μM, about 100 μM, about 125 μM, about 150 μM, about 175 μM, about 200 μM, about 250 μM, about 300 μM, about 350 μM, about 400 μM, about 500 μM, about 600 μM, about 700 μM, about 750 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 400 mM, about 500 mM, about 600 mM, about 700 mM, about 800 mM, about 900 mM, about 1000 mM, about 1 M, about 1.1 M, about 1.2 M, about 1.3 M, about 1.4 M, about 1.5 M, about 1.6 M, about 1.7 M, about 1.8 M, about 1.9 M, about 2 M, about 3 M, about 4 M, about 5 M, about 6 M, about 7 M, about 8 M, about 9 M, about 10 M, about 15 M, about 20 M, about 25 M, about 30 M, about 35 M, about 40 M, about 45 M, about 50 M, about 75 M, about 100 M, or any range in between any two of the aforementioned concentrations, including said two concentrations as endpoints of the range, or any number in between any two of the aforementioned concentrations.

When administered orally, the active agent composition may be protected from digestion. This can be accomplished either by complexing the active agent with a composition to render it resistant to acidic and enzymatic hydrolysis or by packaging the active agent in an appropriately resistant carrier such as (but not limited to) a liposome, e.g., such as shown in U.S. Pat. No. 5,391,377.

In certain non-limiting embodiments, the different therapeutic compounds of the disclosure can be administered within one hour of each other, within two hours of each other, within 3 hours of each other, within 6 hours of each other, within 12 hours of each other, within 24 hours of each other, within 36 hours of each other, within 48 hours of each other, within 72 hours of each other, or more. Thus, an individual who receives such treatment can benefit from a combined effect of the different therapeutic compounds. As noted elsewhere, the dosage of an administered active agent for humans will vary depending upon factors such as (but not limited to) the patient's age, weight, height, sex, general medical condition, and previous medical history. A dosage may be provided as several smaller amounts. For example, a single dosage of 500 mg may be administered as ten 50 mg tablets or capsules, or as five 100 mg tablets or capsules. The amounts of doses or dosages described herein may be provided in a single capsule, tablet, injection, infusion, or other more of delivery. Or, the amounts of drug which comprise the doses or dosages described herein may be provided in two or more (e.g., 2, 3, 4, 5, 6, 7, 8, 9, 10, 11, or 12) capsules, tablets, injections, infusions, or other modes of delivery.

The active agents of the present disclosure can be administered to a subject by any of a number of effective routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compounds may also be formulated for inhalation. In certain non-limiting embodiments, a compound may be simply dissolved or suspended in sterile water. Oral formulations may be formulated such that the active agents passes through a portion of the digestive system before being released, for example it may not be released until reaching the small intestine, or the colon. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

Tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations which are suitable for vaginal administration also include pessaries, tampons, creams, gels, pastes, foams or spray formulations containing such carriers as are known in the art to be appropriate. In certain non-limiting embodiments, the active agents of the present disclosure can be formulated into suppositories, slow-release formulations, or intrauterine delivery devices (IUDs).

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required. The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this disclosure. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A particular (but non-limiting) route of administration is local administration (e.g., topical administration, such as eye drops, or administration via an implant). The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical transdermal administration, the agents are formulated into ointments, creams, salves, powders, and gels. Transdermal delivery systems can also include (for example but not by way of limitation) patches. The present compositions can also be administered in sustained delivery or sustained release mechanisms. For example, biodegradable microspheres or capsules or other biodegradable polymer configurations capable of sustained delivery can be included herein.

The compositions of the present disclosure may be formulated as implants, in the form of either biodegradable microparticles or small squared films comprising the microparticles of the present disclosure. The following describes methods of making such implants. Microparticles (e.g., 5-100 micrometers) containing different loadings of the compounds of the present disclosure may be prepared by spray drying suspensions of the nanocrystals of the compounds and a biodegradable polymer (for example, polylactic acid of molecular weights 50,000-100,000 Da) or polylactic-co-glycolic acid copolymer (e.g., proportions 75:25 or 50:50). Microparticles may contain, e.g., 10-50% wt/wt drug:polymer and can be implanted alone, or in a biodegradable film, e.g., as an implantable chitosan-egg phosphatidylcholine (ePC) films. To make such chitosan-egg phosphatidylcholine (ePC) films, chitosan flakes and ePC can be dissolved in a 1% acetic acid at a ratio of 1:0.8 (wt/wt). Microparticles containing the drug in nanocrystal form are dispersed in the chitosan-ePC solution in different proportions (e.g., 1:3, 1:5, 1:7 and 1:10 wt/wt) to achieve the release of different drug doses. The resulting microparticle-chitosan-ePC suspension can be poured into a Teflon dish to have a 2-3 mm thickness and allowed to dry in a covered dessicator for 5 days. After the films are dry, they can be cut into small squares of 15×15 mm². The implants can be made in different forms, including but not limited to thin films, rods, and wafers. Other biodegradable polymers that can be used to make implants include, but are not limited to, poly-lactic acid, poly-lactic-co-glycolic acid copolymer, poly-caprolactone, poly-sebacic acid, poly-adipic acid, poly (3-hydroxybutyric acid), poly(3-hydroxybutyrate-co-3-hydroxyvalerate, poly-trimethylene carbonate, chitosan, chitin, gelatin, collagen, and hyaluronic acid.

In non-limiting embodiments, gels comprising the active agents of the present disclosure can be made by combining the active agents in various proportions to a sodium alginate gel base or carbomer jelly base to form a homogeneous gel suitable for topical application.

In non-limiting embodiments, ointments comprising the active agents of the present disclosure can be made by combining the active agents in various proportions to a Hydrophilic Petrolatum USP base, Lanolin, USP base or to Polyethylene glycol ointment, NF to form a homogeneous ointment suitable for topical application.

In non-limiting embodiments, creams comprising the active agents of the present disclosure can be made by combining the active agents in various proportions in suspension in water and glycerin (e.g., 20:1 parts) and emulsified in a mixture of e.g., Lanolin, Beeswax USP-NF and Cetyl alcohol, plus a Tween 80 and Span 80.

Several gel, ointment, and/or cream compositions that can be used are shown in Garcia-Contreras L, Abu-Izza K, Lu DR. “Biodegradable cisplatin microspheres for direct brain injection: Preparation and characterization.” Pharm Dev Technol (1997) 2(1): 53-65.

For inhalation, the present compositions can be delivered using any system known in the art, including (but not limited to) dry powder aerosols, liquids delivery systems, air jet nebulizers, propellant systems, and the like. For example (but not by way of limitation), the pharmaceutical formulation can be administered in the form of an aerosol or mist. For aerosol administration, the formulation can be supplied in finely divided form along with a surfactant and propellant. In another non-limiting aspect, the device for delivering the formulation to respiratory tissue is an inhaler in which the formulation vaporizes. Other liquid delivery systems include (for example but not by way of limitation) air jet nebulizers.

For inhalation, the present compositions can be delivered using any system known in the art, including (but not limited to) dry powder aerosols, liquids delivery systems, air jet nebulizers, propellant systems, and the like. For example (but not by way of limitation), the pharmaceutical formulation can be administered in the form of an aerosol or mist. For aerosol administration, the formulation can be supplied in finely divided form along with a surfactant and propellant. In another non-limiting aspect, the device for delivering the formulation to respiratory tissue is an inhaler in which the formulation vaporizes. Other liquid delivery systems include (for example, but not by way of limitation) air jet nebulizers.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, to prolong the effect of the active agent, it is desirable to slow the absorption of the active agent from subcutaneous or intramuscular injection. This may be accomplished by using a liquid suspension of crystalline or amorphous material having poor water solubility. The rate of absorption of the active agent then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the active agent(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

As noted, effective amounts of the active agents may be administered orally, in the form of a solid or liquid preparations such as capsules, pills, tablets, lozenges, melts, powders, suspensions, solutions, elixirs or emulsions. Solid unit dosage forms can be capsules of the ordinary gelatin type containing, for example, surfactants, lubricants, and inert fillers such as lactose, sucrose, and cornstarch, or the dosage forms can be sustained release preparations. The pharmaceutical composition may contain a solid carrier, such as a gelatin or an adjuvant. The tablet, capsule, and powder may contain from about 0.05 to about 95% of the active substance compound by dry weight. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol, or polyethylene glycol. When administered in liquid form, the pharmaceutical composition particularly contains from about 0.005 to about 95% by weight of the active agent(s). For example, a dose of about 10 mg to about 1000 mg once or twice a day could be administered orally.

In another non-limiting embodiment, the active agents of the present disclosure can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the active agents in an aqueous or non-aqueous pharmaceutically acceptable solvent which may also contain suspending agents, sweetening agents, flavoring agents, and preservative agents as are known in the art.

In one non-limiting aspect, the active agent is incorporated in lipid monolayers or bilayers, such as (but not limited to) liposomes. Liposomes and liposomal formulations can be prepared according to standard methods and are also well known in the art, such as (but not limited to) those disclosed in U.S. Pat. Nos. 6,110,490; 6,096,716; 5,283,185; 5,279,833; 4,235,871; 4,501,728; and 4,837,028.

In one non-limiting aspect, the compositions are prepared with carriers that will protect the active agent against rapid elimination from the body, such as (but not limited to) a controlled release formulation, including PEGylation, lipids, implants and microencapsulated delivery systems. Biodegradable, biocompatible polymers can be used, such as (but not limited to) ethylene vinyl acetate, polyanhydrides, polyglycolic acid, collagen, polyorthoesters, and polylactic acid. Methods for preparation of such formulations will be apparent to those skilled in the art.

The active agents in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active agent is combined in a mixture with one or more pharmaceutically suitable excipients. Sterile phosphate-buffered saline is one non-limiting example of a pharmaceutically suitable excipient.

The acid addition salts may include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N?-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

As noted elsewhere herein, non-limiting examples of routes of administration of the compositions described herein include parenteral injection, e.g., by subcutaneous, intramuscular, or transdermal delivery. Other forms of injection include (but are not limited to) intravenous, intraarterial, intralymphatic, intrathecal, intraocular, intranasal, intracranial, intracerebral, intraperitoneal, or intracavitary injection. In parenteral administration, the compositions will be formulated in a unit dosage injectable form such as (but not limited to) a solution, suspension, or emulsion, in association with a pharmaceutically acceptable excipient. Such excipients are inherently nontoxic and nontherapeutic. Non-limiting examples of such excipients include saline, Ringer's solution, dextrose solution, and Hanks' solution. Nonaqueous excipients such as (but not limited to) fixed oils and ethyl oleate may also be used. An alternative non-limiting excipient is 5% dextrose in saline. The excipient may contain minor amounts of additives such as (but not limited to) substances that enhance isotonicity and chemical stability, including buffers and preservatives. The active agents can be delivered or administered alone or as pharmaceutical compositions by any means known in the art, such as (but not limited to) systemically, regionally, or locally, for example by intraarterial, intrathecal (IT), intravenous (IV), parenteral, intrapleural cavity, topical, oral, or local administration, as subcutaneous, intratracheal (e.g., by aerosol) or transmucosal administration (e.g., buccal, bladder, vaginal, uterine, rectal, and/or nasal mucosa). Administration can also be localized directly into a tumor. Administration into the systemic circulation by intravenous, inhalation, mucosal, or subcutaneous administration is typical. Intravenous administration can be, for example (but not by way of limitation), by infusion over a period such as (but not limited to) 30-90 min or by a single bolus injection, or by other regimens as described elsewhere herein.

For parenteral administration, for example, the active agents may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives and buffers as are known in the art.

When an effective amount of the active agents is administered by intravenous, cutaneous, or subcutaneous injection, the compound is particularly in the form of a pyrogen-free, parenterally acceptable aqueous solution or suspension. The preparation of such parenterally acceptable solutions, having due regard to pH, isotonicity, stability, and the like, is well within the skill in the art. A particular pharmaceutical composition for intravenous, cutaneous, or subcutaneous injection may contain, in addition to the active agent, an isotonic vehicle such as Sodium Chloride Injection, Ringer's Injection, Dextrose Injection, Dextrose and Sodium Chloride Injection, Lactated Ringer's Injection, or other vehicles as known in the art. The pharmaceutical compositions of the present disclosure may also contain stabilizers, preservatives, buffers, antioxidants, or other additives known to those of skill in the art.

As noted, particular amounts and modes of administration can be determined by one skilled in the art. One skilled in the art of preparing formulations can readily select the proper form and mode of administration, depending upon the particular characteristics of the active agents selected, the condition to be treated, the stage of the condition, and other relevant circumstances using formulation technology known in the art, described, for example, in Remington: The Science and Practice of Pharmacy, 22nd ed.

Additional pharmaceutical methods may be employed to control the duration of action of the active agents. Increased half-life and/or controlled release preparations may be achieved by using proteins or polymers to conjugate, complex with, and/or absorb the active agents as discussed previously herein. The controlled delivery and/or increased half-life may be achieved by selecting appropriate macromolecules (for example but not by way of limitation, lipids, polysaccharides, polyesters, polyamino acids, homopolymers, polyvinyl pyrrolidone, ethylenevinylacetate, methylcellulose, or carboxymethylcellulose, and acrylamides such as N-(2-hydroxypropyl) methacrylamide), and the appropriate concentration of macromolecules as well as the methods of incorporation, in order to control release.

Another possible method useful in controlling the duration of action of the active agents by controlled release preparations and half-life is incorporation of the active agents or their functional derivatives into particles of a polymeric material such as polyesters, polyamides, polyamino acids, hydrogels, poly(lactic acid), ethylene vinylacetate copolymers, copolymer micelles of, for example, polyethylene glycol (PEG) and poly(1-aspartamide).

It is also possible to entrap the active agents in microcapsules prepared, for example, by coacervation techniques or by interfacial polymerization (for example, hydroxymethylcellulose or gelatine-microcapsules and poly-(methylmethacrylate) microcapsules, respectively), in colloidal drug delivery systems (for example, liposomes, albumin microspheres, microemulsions, nanoparticles, and nanocapsules), or in macroemulsions. Such techniques are well known to persons having ordinary skill in the art.

The peptides of the present disclosure can be modified by processes including, but not limited to, lipidation e.g., palmitoylation, PEGylation, lactamization, and disulfide bridge closure. Lipidation creates a circulating reservoir of peptide which reversibly associates with naturally abundant albumin in blood serum. Peptide associated with albumin effectively escapes renal ultrafiltration since the size of the associated complex is above the glomerular filtration cutoff. As the peptide dissociates from the surface of the albumin it is again free to interact with endogenous receptors. PEGylation physically shields the peptide from proteolysis and imparts significant hydrophilicity which upon hydration greatly increases the hydrodynamic radius of the therapeutic molecule to overcome renal clearance. These technologies are thus able to extend the circulatory half-life of the peptides.

The terms “lipid modified amino acid” and “lipidated amino acid” are used interchangeably herein, and refer to an amino acid, typically a lysine or cysteine, which has a lipid moiety attached. The terms “lipidated peptide,” “lipidated polypeptide,” “lipoprotein,” and the like refer to a peptide or polypeptide that includes one or more lipid-modified amino acids.

Lipidation, in the present embodiments, can include N-terminal lipidation, main-chain lipidation, and/or side-chain lipidation. In certain non-limiting embodiments, the modification is of a non-terminal main-chain amino acid residue. The peptides may be lipidated at more than one position. In certain non-limiting embodiments, improvements in protease resistance and increased potency are associated with the selective and strategic position of the lipidation of one or more main-chain amino acid residues. Methods of preparing peptides with lipid modified amino acids are known in the art.

As used herein, the term “lipid” refers to any fatty, waxy, or other nonpolar compound (e.g., cholesterol) that is readily soluble in a nonpolar solvent (e.g., 2-octanol or a hydrocarbon solvent like n-hexane), but which is substantially insoluble in a polar solvent (e.g., water). Examples of lipids include, but are not limited to, oils, fats, waxes, isoprenoids, gangliosides, and steroids. In some non-limiting embodiments, the lipid entity is selected from the group consisting of fatty acids, sterols, polyethers, pepducins, gangliosides, sphingolipids, glycosphingolipids, and ceramides. Exemplary lipids which may be used for lipidation herein include, but are not limited to, myristic acid, palmitic acid, stearic acid, palmitoleic acid, oleic acid, α-linolenic acid, eicosapentaenoic acid (EPA), docosahexaenoic acid (DHA), cholesterol, 1,2-bis(diphenylphosphino)ethane (DPPE), dipalmitoylphosphatidylcholine (DPPC), dioleoylphophatidylserine (DOPS), dioleoylphophatidylcholine (DOPC), sphingolipids such as gangliosides, ceramides, monosialotetrahexosylganglioside (GM1) and other gangliosides (e.g., GM2, GM3, etc.), fluorinated gangliosides (e.g., fluorinated-GM1, fluorinated-GM2, fluorinated-GM3, etc.). In some non-limiting embodiments, a lipid entity is palmitic acid. In some non-limiting embodiments, a lipid entity is the ganglioside GM₁. In some non-limiting embodiments, lipidation may comprise myristoylation. As used herein, “myristoylation” refers to the attachment of a myristate to an amino group of an amino acid. In some non-limiting embodiments, lipidation may comprise palmitoylation. As used herein “palmitoylation” refers to the creation of a thioester linkage of long-chain fatty acids on one or more cysteine residues present in a peptide or protein.

In certain non-limiting embodiments, a lipidated peptide comprising at least one lipidated amino acid residue is provided. In certain non-limiting embodiments, the lipidated peptide comprises at least two lipidated amino acid residues. In certain non-limiting embodiments, the lipidated peptide contains only one lipidated amino acid residue. As used herein, a peptide with one lipid or lipid moiety attached is referred to as a mono-lipidated peptide. In other non-limiting embodiments, the lipidated peptide contains two lipidated amino acid residues. As used herein, a peptide with two lipids or lipid moieties attached is referred to as a bis-lipidated peptide.

In certain non-limiting embodiments, the lipidated synthetic peptide comprises at least one substitution of an alpha-methyl functionalized amino acid for a native amino acid residue. In other non-limiting embodiments, a lipidated synthetic peptide comprises at least two, three, four, five, six, or more substitutions of alpha-methyl (α-met) functionalized amino acids for native amino acid residues. As described throughout, α-met functionalized amino acids can replace any native amino acid in a peptide. The term “native” amino acid refers to one of the standard 20 amino acids that exist in biologically generated proteins.

In certain non-limiting embodiments, a lipidated peptide as provided herein can also comprise one or more α-met functionalized amino acids corresponding to the substituted native amino acids in a corresponding wild-type protein. For example, the amino acid in the original, wild-type peptide sequence can be substituted with an α-met functionalized amino acid that has the same side chain, e.g., Phe, Trp, Tyr, Ser, Arg, Ala, Val, Leu, His, or Lys, can be substituted with α-MePhe, α-MeTrp, α-MeTyr, α-MeSer, α-MeArg, α-MeAla, α-MeVal, α-MeLeu, α-MeHis, or α-MeLys, respectively.

In certain non-limiting embodiments, an α-met functionalized amino acid in a lipidated peptide as provided herein can correspond to the same class as the substituted native amino acids. For example, aliphatic α-met functionalized amino acids can be substituted for aliphatic native amino acids; hydroxyl α-met functionalized amino acids can be substituted for hydroxyl native amino acids; sulfur-containing α-met functionalized amino acids can be substituted for sulfur-containing native amino acids; cyclic α-met functionalized amino acids can be substituted for cyclic native amino acids; aromatic α-met functionalized amino acids can be substituted for aromatic native amino acids; basic α-met functionalized amino acids can be substituted for basic native amino acids; and/or acidic α-met functionalized amino acids can be substituted for acidic native amino acids.

In certain non-limiting embodiments, at least one α-met functionalized amino acid in a lipidated synthetic peptide described herein is α-met phenylalanine. In certain non-limiting embodiments, at least one α-met functionalized amino acid in a synthetic lipidated peptide described herein is selected from α-met functionalized Histidine, α-met functionalized Alanine, α-met functionalized Isoleucine, α-met functionalized Arginine, α-met functionalized Leucine, α-met functionalized Asparagine, α-met functionalized Lysine, α-met functionalized Aspartic acid, α-met functionalized Methionine, α-met functionalized Cysteine, α-met functionalized Phenylalanine, α-met functionalized Glutamic acid, α-met functionalized Threonine, α-met functionalized Glutamine, α-met functionalized Tryptophan, α-met functionalized Glycine, α-met functionalized Valine, α-met functionalized Ornithine, α-met functionalized Praline, α-met functionalized Selenocysteine, α-met functionalized Serine, and α-met functionalized Tyrosine.

In some non-limiting embodiments, lipidation comprises GPI-anchor addition. As used herein “GPI-anchor addition” refers to the linkage of glycosyl-phosphatidylinositol (GPI) to the C-terminus of a peptide. In some non-limiting embodiments, lipidation comprises prenylation. As used herein “prenylation” refers to the creation of a thioether linkage of an isoprenoid lipid (e.g., farnesyl (C-15) or geranylgeranyl (C-20)) to a cysteine present in a peptide or protein. In some non-limiting embodiments, lipidation comprises geranylation. In some non-limiting embodiments, lipidation comprises the association of a ligand entity with any compound that is soluble in a cellular membrane.

When the active agents are to be used as an injectable material, they can be formulated into a conventional injectable carrier. Suitable carriers include biocompatible and pharmaceutically acceptable phosphate buffered saline solutions, which are particularly isotonic.

For reconstitution of a lyophilized product in accordance with the present disclosure, one may employ a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which are available to those in the field. The pharmaceutical composition may also be in the form of an aqueous solution containing many of the same substances as described above for the reconstitution of a lyophilized product.

The active agents can also be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, tauric acid, maleic acid, and fumaric acid, or by reaction with an inorganic base such as sodium hydroxide, ammonium hydroxide, potassium hydroxide, and organic bases such as monoalkyl, dialkyl, trialkyl and aryl amines, and substituted ethanolamines.

As described elsewhere herein, in certain non-limiting embodiments, the present disclosure includes an active agent composition wherein at least one of the active agents is coupled (e.g., by covalent bond) directly or indirectly to a carrier molecule.

Formulated compositions comprising the active agents of the present disclosure can be provided in unit dosage form, e.g., in ampoules or in multi-dose containers, with an added preservative. Compositions can also take such forms as suspensions, solutions, or emulsions in oily or aqueous vehicles, and can contain formulatory agents such as suspending, stabilizing, and/or dispersing agents.

In some non-limiting methods, the patient is administered the active agent every one, two, three, or four weeks, for example. The dosage depends on the frequency of administration, condition of the patient, response to prior treatment (if any), whether the treatment is prophylactic or therapeutic, and whether the disorder is acute or chronic, among other factors.

The number of dosages administered may depends on the severity and temporal nature of the disorder (e.g., whether presenting acute or chronic symptoms) and the response of the disorder to the treatment. For acute disorders or acute exacerbations of a chronic disorder, between 1 and 10 doses may be used. Sometimes a single bolus dose, optionally in divided form, is sufficient for an acute disorder or acute exacerbation of a chronic disorder. Treatment can be repeated for recurrence of an acute disorder or acute exacerbation. For chronic disorders, the active agent may be administered at regular intervals, such as (but not limited to) weekly, fortnightly, monthly, quarterly, every six months for at least 1, 5, or 10 years, or for the life of the patient.

In at least certain non-limiting embodiments, when the active agent is provided in the form of a capsule or tablet, the capsule or tablet should disintegrate within about 5-10 minutes, and in the gastrointestinal (GI) tract the active agent should dissolve in about 30 minutes.

Where used herein, the term CLR:RAMP1 refers to the CGRP receptor, CLR:RAMP2 refers to the AM₁ (AM1) receptor, and CLR:RAMP3 refers to the AM₂ (AM2) receptor. The AM₁ receptor may also be referred to herein as AMIR or AM₁R. The AM₂ receptor may also be referred to herein as AM₂R or AM₂R. As noted above, AM2 is the same as IMD.

Where used herein the term “fragment” refers to a portion of a complete amino acid sequence or nucleic acid sequence. For example, a fragment of wild type AM refers, in at least certain non-limiting embodiments, to a peptide having 5 or more contiguous amino acids of wild type AM, for example, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 amino acids.

A fragment of wild type AM2 refers, in at least certain non-limiting embodiments, to a peptide having 5 or more contiguous amino acids of wild type AM2, for example, 5, 6, 7, 8, 9, 10, 11, 12 13, 14, 15, 16, 17, 18, 19, 20, 21, 22, 23, 24, 25, 26, 27, 28, 29, 30, 31, or 32 amino acids.

A fragment of wild type CGRP refers, in at least certain non-limiting embodiments, to a peptide having 4 or more contiguous amino acids of wild type α-CGRP or wild type j-CGRP, for example, 4, 5, 6, 7, 8, 9, 10, 11, 12 13, 14, 15, 16, 17, 18, 19, 20, 21, 22, 23, 24, 25, 26, 27, 28, 29, 30, 31, 32, or 33 amino acids.

Non-limiting embodiments of variant peptides of the present disclosure are included in specific examples shown below, which comprise just a small subset of the variant peptides supported and enabled herein. Unless otherwise specified, the C-terminal residue of the variant peptides is amidated (i.e., the C-terminal OH group is replaced by NH₂). In certain non-limiting embodiments, a blocking group, such as an acetyl or a lipid, is linked to the N-terminal residue amino acid. Three wild-type forms of AM2 have been isolated in vivo, a 53-residue form, a 47-residue form, and a 40-residue form (described below). In non-limiting embodiments, a variety of various peptide sequences of the present disclosure are shown below and in Tables 1-6 and 12-20.

Wild-type human adrenomedullin, referred to herein in some non-limiting embodiments as AM(1-52)NH₂, is an agonistic 52-amino acid, C-terminally-amidated peptide having the sequence:

(SEQ ID NO: 1)
YRQSMNNFQGLRSFGCRFGTCTVQKLAHQIYQFTDKDKDNVAPRSKISPQ
GY-NH₂,

with a disulfide bond between C16 and C21. Amino acids 1-21 and 1-22 of SEQ ID NO:1 are shown as underlined and boldfaced, respectively.

AM(13-52)NH₂ is an agonistic truncated fragment of SEQ ID NO:1 having the sequence:

(SEQ ID NO: 2)
SFGCRFGTCTVQKLAHQIYQFTDKDKDNVAPRSKISPQGY-NH₂,

disulfide bond between the two cysteines. Amino acids 1-9 or 1-10 of SEQ ID NO:2 are shown as underlined and boldfaced, respectively.

A variant of AM(13-52) referred to herein as AM(13-52)NH₂[S48G, Q50W], has the sequence SFGCRFGTCTVQKLAHQIYQFTDKDKDNVAPRSKIGPWGY-NH₂ (SEQ ID NO:52).

AM(22-52)NH₂ is an antagonistic truncated fragment of SEQ ID NO:1 having the sequence: TVQKLAHQIYQFTDKDKDNVAPRSKISPQGY-NH₂ (SEQ ID NO:3).

AM(37-52)NH₂ is an antagonistic truncated fragment of SEQ ID NO:1 having the sequence: DKDNVAPRSKISPQGY-NH₂ (SEQ ID NO:4).

Where substitutions are made in AM, or in any portion thereof (such as AM(37-52)) sequence to form variants, in the notation used to refer to the substitutions in the variants, such as S45W, K46L, S48G, Q50W, and Y52F, the positions refer to the positions (45, 46, 48, 50, 52) in the wild-type AM, not the number of the actual position in the variant peptide. For example in the 40-amino acid variant called AM(13-52)NH₂[S48G, Q50W], the substitution referred to as “S48G” is actually made in position 38 of the variant, and the substitution referred to as “Q50W” is actually made in position 40 of the variant.

As noted above, wild-type human AM2 can be found in three forms, as described below.

The first human AM2, referred to herein in some non-limiting embodiments as 2(1-53)NH₂, is an agonistic, 53-amino acid C-terminally-amidated peptide having the sequence:

(SEQ ID NO: 5)
HSGPRRTQAQLLRVGCVLGTCQVQNLSHRLWQLMGPAGRQDSAPVDPS
SPHSY-NH₂,

with a disulfide bond between the two cysteines. Amino acids 1-21 or 1-22 of SEQ ID NO:5 are shown as underlined and boldfaced, respectively.

The second human AM2, referred to herein in some non-limiting embodiments as AM2(1-47)NH₂ is an agonistic, 47-amino acid, C-terminally-amidated peptide having the sequence:

(SEQ ID NO: 6)
TQAQLLRVGCVLGTCQVQNLSHRLWQLMGPAGRQDSAPVDPSSPHSY-
NH₂,

with a disulfide bond between the two cysteines. Amino acids 1-15 or 1-16 of SEQ ID NO:6 are shown as underlined and boldfaced, respectively.

The third human AM2, referred to herein in some non-limiting embodiments as AM2 (8-47)NH₂, is an agonistic, 40-amino acid, C-terminally-amidated peptide having the sequence:

(SEQ ID NO: 7)
VGCVLGTCQVQNLSHRLWQLMGPAGRQDSAPVDPSSPHSY-NH₂,

with a disulfide bond between the two cysteines. Amino acids 1-8 or 1-9 of SEQ ID NO:7 are shown as underlined and boldfaced, respectively.

An AM2 fragment, AM2(17-47)NH₂ is an antagonistic, 31-amino acid, N-terminally acetylated and C-terminally amidated peptide fragment of SEQ ID NO:6 having the sequence:

(SEQ ID NO: 8)
Ac-VQNLSHRLWQLMGPAGRQDSAPVDPSSPHSY-NH₂.

Wild-type human Calcitonin gene-related peptide, referred to herein in some non-limiting embodiments as ha-CGRP(1-37)NH₂, is an agonistic, 37-amino acid, C-terminally-amidated peptide having the sequence:

(SEQ ID NO: 9)
ACDTATCVTHRLAGLLSRSGGVVKNNFVPTNVGSKAF-NH₂,

with a disulfide bond between the two cysteines. Amino acids 1-7 or 1-8 of SEQ ID NO:9 are shown as underlined and boldfaced, respectively.

Truncation of the first 7 amino acids of hα-CGRP(1-37)NH₂ provides α-CGRP(8-37)NH₂, an antagonistic 30-amino acid peptide having the sequence:

(SEQ ID NO: 10)
VTHRLAGLLSRSGGVVKNNFVPTNVGSKAF-NH₂.

Truncation of the first 26 amino acids of hα-CGRP(1-37)N1H2 provides α-CGRP(27-37)NH₂, an antagonistic 11-amino acid peptide having the sequence: FVPTNVGSKAF-NH₂ (SEQ ID NO:11), which was previously published in Rist et al. (J Med Chem. (1998) 41(1):117-23).

Wild-type human Calcitonin gene-related peptide, referred to herein in some non-limiting embodiments as hβ-CGRP(1-37)NH₂, is an agonistic 37-amino acid, C-terminally-amidated peptide having the sequence:

(SEQ ID NO: 12)
ACNTATCVTHRLAGLLSRSGGMVKSNFVPTNVGSKAF-NH₂,

with a disulfide bond between the two cysteines. Amino acids 1-7 or 1-8 of SEQ ID NO:12 are shown as underlined and boldfaced, respectively.

Wild type human AMY, referred to herein in some non-limiting embodiments as hAMY(1-37)NH₂, is an agonistic 37-amino acid, C-terminally-amidated peptide having the sequence:

(SEQ ID NO: 13)
KCNTATCATQRLANFLVHSSNNFGAILSSTNVGSNTY-NH₂,

with a disulfide bond between the two cysteines. Amino acids 1-7 or 1-8 of SEQ ID NO:13 are shown as underlined and boldfaced, respectively.

Truncation of the first 7 amino acids of hAMY(1-37)NH₂ provides hAMY(8-37)NH₂, an antagonistic 30-amino acid peptide having the sequence:

(SEQ ID NO: 14)
ATQRLANFLVHSSNNFGAILSSTNVGSNTY-NH₂.

Wild-type human CT, referred to herein in some non-limiting embodiments as hCT(1-32)NH₂, is an agonistic, 32-amino acid, C-terminally-amidated peptide having the sequence:

(SEQ ID NO: 15)
CGNLSTCMLGTYTQDFNKFHTFPQTAIGVGAP-NH₂,

with a disulfide bond between the two cysteines. Amino acids 1-7 or 1-8 of SEQ ID NO: 15 are shown as underlined and boldfaced, respectively.

Truncation of the first 7 amino acids of hCT(1-32)NH₂ provides hCT(8-32)NH₂, an antagonistic 25-amino acid peptide having the sequence:

(SEQ ID NO: 16)
MLGTYTQDFNKFHTFPQTAIGVGAP-NH₂.

sCT(1-32)NH₂ is an agonistic, 32-amino acid, C-terminally-amidated peptide having the sequence:

(SEQ ID NO: 17)
CSNLSTCVLGKLSQELHKLQTYPRTNTGSGTP-NH₂,

with a disulfide bond between the two cysteines. Amino acids 1-7 or 1-8 of SEQ ID NO:17 are shown as underlined and boldfaced, respectively.

Truncation of the first 7 amino acids of sCT(1-32)NH₂ provides sCT(8-32)NH₂, an antagonistic 25-amino acid peptide having the sequence:

(SEQ ID NO: 18)
VLGKLSQELHKLQTYPRTNTGSGTP-NH₂.

In certain non-limiting embodiments, the peptide is derived from AM2 and comprises the amino acid sequence X₁QNLSHX₇LWQLMGPAG X₁₇QDSAPVDPSSP X₂₉SX₃₁ (SEQ ID NO:19), wherein
X₁ is V, wherein the V optionally comprises a first blocking group,
X₇ is R, K, Q, L, A, or S,
X₁₇ is R, K, A, S, or N,
X₂₉ is W, H, Q, Y, F, R, K, I, L, or M, and
X₃₁ is Y or F, wherein the Y or F comprises a second blocking group. In certain non-limiting embodiments, the first blocking group is an acetyl, such that the V is acetylated, or a lipid, such that the V is lipidated, and the second blocking group is an amine such that X₃₁ is amidated.

In one version of SEQ ID NO: 19, wherein X₇ is R, X₁₇ is R, X₂₉ is W, and X₃₁ is Y, the peptide is designated herein as AM2(17-47)NH₂ [H45W], and comprises the sequence:

(SEQ ID NO: 20)
VQNLSHRLWQLMGPAGRQDSAPVDPSSPWSY

In certain non-limiting embodiments, the peptide is derived from portions of AM2 and AM and comprises the amino acid sequence:
X₁X₂X₃LX₅X₆RLWQLMGPAGRDNVAPRX₂₄X₂₅IX₂₇PX₂₉GX₃₁ (SEQ ID NO:21), wherein
X₁ is V, T, or L, and wherein the V, T, or L optionally comprises a first blocking group,
X₂ is Q, H, or G,
X₃ is N, K, R, or T,
X₅ is S, A, or T,
X₆ is H, G, N, or Q,
X₂₄ is S, W, F, Y, or R,
X₂₅ is K, L, R, Q, Y, I, V, M, A, or G,
X₂₇ is S or G,
X₂₉ is Q, W, Y, F, R, K, I, L, or M, and
X₃₁ is Y or F, and wherein the Y or F comprises a second blocking group.

Non-limiting embodiments of variant peptides of SEQ ID NO:21 comprise the amino acid sequences SEQ ID NOS:22-25, and 57-71 (see Table 3).

Further non-limiting examples of variants of SEQ ID NO:21 to which the present disclosure is directed are shown in Tables 12-20.

In certain non-limiting embodiments, the peptide is derived from portions of AM and AM2 and comprises the amino acid sequence:
X₁X₂X₃ X₄LX₆X₇RLWQLMGPAGRDNVAPRX₂₅X₂₆IX₂₈PX₃₀GX₃₂ (SEQ ID NO:26), wherein
X₁ is T, V, A or M, and wherein the T, V, A or M optionally comprises a first blocking group,
X₂ is V, T, or L,
X₃ is Q, H, or G,
X₄ is K, N, R, or T,
X₆ is A, S, or T,
X₇ is H, N, G, or Q,
X₂₅ is S, W, F, Y, or R,
X₂₆ is K, L, R, Q, Y, I, V, M, A, or G,
X₂₈ is S or G,
X₃₀ is Q, W, F, Y, R, K, I, L, or M, and
X₃₂ is Y or F, and wherein the Y or F comprises a second blocking group. In certain non-limiting embodiments, the first blocking group is an acetyl, such that the V is acetylated, or a lipid, such that the V is lipidated, and the second blocking group is an amine such that X₃₁ is amidated.

Non-limiting embodiments of variant peptides of SEQ ID NO:26 comprise the amino acid sequences SEQ ID NOS:27-30, and 72-86 (see Table 4).

In certain non-limiting embodiments, the peptide is derived from portions of CGRP, AM and AM2 and comprises the amino acid sequence:
X₁THRLAGRLWQLMGPAGRDNVAPRX₂₅X₂₆IX₂₈PX₃₀GX₃₂ (SEQ ID NO:31), wherein
X₁ is V, and optionally comprises a first blocking group,
X₂₅ is S, W, F, Y, or R,
X₂₆ is K, L, R, Q, Y, I, V, M, A, or G,
X₂₈ is S or G,
X₃₀ is Q, W, F, Y, R, K, I, L, or M, and
X₃₂ is Y or F, and wherein the Y or F comprises a second blocking group. In certain non-limiting embodiments, the first blocking group is an acetyl, such that the V is acetylated, or a lipid, such that the V is lipidated, and the second blocking group is an amine such that X₃₁ is amidated.

In one non-limiting embodiment of SEQ ID NO:31, wherein X₂₅ is S, X₂₆ is K, X₂₈ is S, X₃₀ is Q, and X₃₂ is Y, the peptide comprises the amino acid sequence: VTHRLAGRLWQLMGPAGRDNVAPRSKISPQGY (SEQ ID NO:32) and is designated herein as CGRP(8-14)-AM2(23-33)-AM(39-52)NH₂.

In certain non-limiting embodiments, the peptide is derived from portions of AMY, AM and AM2 and comprises the amino acid sequence:
X₁TQRLANRLWQLMGPAGRDNVAPRX₂₅X₂₆IX₂₈PX₃₀GX₃₂ (SEQ ID NO:33), wherein
X₁ is A, and optionally is linked to a first blocking group,
X₂₅ is S, W, F, Y, or R,
X₂₆ is K, L, R, Q, Y, I, V, M, A, or G,
X₂₈ is S or G,
X₃₀ is Q, W, F, Y, R, K, I, L, or M, and
X₃₂ is Y or F, and wherein the Y or F comprises a second blocking group. In certain non-limiting embodiments, the first blocking group is an acetyl, such that the V is acetylated, or a lipid, such that the V is lipidated, and the second blocking group is an amine such that X₃₁ is amidated.

In one non-limiting embodiment of SEQ ID NO:33, wherein X₂₅ is S, X₂₆ is K, X₂₈ is S, X₃₀ is Q, and X₃₂ is Y, the peptide comprises the amino acid sequence:
ATQRLANRLWQLMGPAGRDNVAPRSKISPQGY (SEQ ID NO:34) and is designated herein as AMY(8-14)-AM2(23-33)-AM(39-52)NH₂.

In certain non-limiting embodiments, the peptide is derived from portions of hCT, AM and AM2 and comprises the amino acid sequence:
X₁LGTYTQRLWQLMGPAGRDNVAPRX₂₅X₂₆IX₂₈PX₃₀GX₃₂ (SEQ ID NO:35), wherein
X₁ is M, and optionally is linked to a first blocking group,
X₂₅ is S, W, F, Y, or R,
X₂₆ is K, L, R, Q, Y, I, V, M, A, or G,
X₂₈ is S or G,
X₃₀ is Q, W, F, Y, R, K, I, L, or M, and
X₃₂ is Y or F, and wherein the Y or F comprises a second blocking group. In certain non-limiting embodiments, the first blocking group is an acetyl, such that the V is acetylated, or a lipid, such that the V is lipidated, and the second blocking group is an amine such that X₃₁ is amidated.

In one non-limiting embodiment of SEQ ID NO:35, wherein X₂₅ is S, X₂₆ is K, X₂₈ is S, X₃₀ is Q, and X₃₂ is Y, the peptide comprises the amino acid sequence:
MLGTYTQRLWQLMGPAGRDNVAPRSKISPQGY (SEQ ID NO:36) and is designated herein as hCT(8-14)-AM2(23-33)-AM(39-52)NH₂.

In certain non-limiting embodiments, the peptide is derived from portions of sCT, AM and AM2 and comprises the amino acid sequence:
X₁LGTYTQRLWQLMGPAGRDNVAPRX₂₅X₂₆IX₂₈PX₃₀GX₃₂ (SEQ ID NO:37), wherein
X₁ is V, and optionally is linked to a first blocking group,
X₂₅ is S, W, F, Y, or R,
X₂₆ is K, L, R, Q, Y, I, V, M, A, or G,
X₂₈ is S or G,
X₃₀ is Q, W, F, Y, R, K, I, L, or M, and
X₃₂ is Y or F, and wherein the Y or F comprises a second blocking group. In certain non-limiting embodiments, the first blocking group is an acetyl, such that the V is acetylated, or a lipid, such that the V is lipidated, and the second blocking group is an amine such that X₃₁ is amidated.

In one non-limiting embodiment of SEQ ID NO:37, wherein X₂₅ is S, X₂₆ is K, X₂₈ is S, X₃₀ is Q, and X₃₂ is Y, the peptide comprises the amino acid sequence:
VLGTYTQRLWQLMGPAGRDNVAPRSKISPQGY (SEQ ID NO:38) and is designated herein as sCT(8-14)-AM2(23-33)-AM(39-52)NH₂.

Other examples of agonistic peptides include, but are not limited to, SEQ ID NOS: 8, 19, 20 21, 22, 23, 24, and 25 which have been extended at the (unblocked)N-terminal end by an amino acid sequence selected from amino acids 1-22 of SEQ ID NO:1, or by amino acids 1-10 of SEQ ID NO:2, or by amino acids 1-22 of SEQ ID NO:5, or by amino acids 1-16 of SEQ ID NO:6, or by amino acids 1-9 of SEQ ID NO:7, or by amino acids 1-8 of SEQ ID NO:9, or by amino acids 1-8 of SEQ ID NO:12, or by amino acids 1-8 of SEQ ID NO:13, or by amino acids 1-8 of SEQ ID NO:15, or by amino acids 1-8 of SEQ ID NO:17. Any of these combined sequences optionally comprises a blocking group (e.g., a lipid or an acetyl) linked to the N-terminal amino acid.

For example, SEQ ID NO:8 when N-terminally extended by amino acids 1-22 of SEQ ID NO:1 has the amino acid sequence:
YRQSMNNFQGLRSFGCRFGTCTVQNLSHRLWQLMGPAGRQDSAPVDPSSPHSY-NH₂ (SEQ ID NO: 39), wherein the sequence in boldface is amino acids 1-22 of SEQ ID NO:1.

Other examples of agonistic peptides include, but are not limited to, SEQ ID NOS: 26-38 which have been extended at the (unblocked)N-terminal end by an amino acid sequence selected from amino acids 1-21 of SEQ ID NO:1, amino acids 1-9 of SEQ ID NO:2, amino acids 1-21 of SEQ ID NO:5, amino acids 1-15 of SEQ ID NO:6, amino acids 1-8 of SEQ ID NO:7, amino acids 1-7 of SEQ ID NO:9, amino acids 1-7 of SEQ ID NO:12, amino acids 1-7 of SEQ ID NO:13, amino acids 1-7 of SEQ ID NO: 15, and amino acids 1-7 of SEQ ID NO: 17. Any of these combined sequences optionally comprises a blocking group (e.g., a lipid or an acetyl) linked to the N-terminal amino acid.

For example, SEQ ID NO:27 when N-terminally extended by amino acids 1-21 of SEQ ID NO:1 has the amino acid sequence:

(SEQ ID NO: 40)
YRQSMNNFQGLRSFGCRFGTCTVQKLAHRLWQLMGPAGRDNVAPRSKISPQ
GY,

wherein the underlined sequence is amino acids 1-21 of SEQ ID NO:1.

Other specific variant peptides which may be used in the methods of the present disclosure include, but are not limited to, those shown in Table 5 (variants of peptide AM(13-28)-AM2(23-33)-AM(39-52)) and Table 6 (variants of peptide AM2(8-33)-AM(39-52)).

TABLE 1
Peptides

| Identifier | Amino acid sequence (SEQ ID NO) |
|---|---|
| AM(1-52)NH₂ | YRQSMNNFQGLRSFGCRFGTCTVQKLAHQIYQFTDKDKDNV |
| | APRSKISPQGY-NH₂ (SEQ ID NO: 1) |
| AM(13-52)NH₂ | SFGCRFGTCTVQKLAHQIYQFTDKDKDNVAPRSKISPQGY- |
| | NH₂ (SEQ ID NO: 2) |
| AM(22-52)NH₂ | TVQKLAHQIYQFTDKDKDNVAPRSKISPQGY-NH₂ |
| | (SEQ ID NO: 3) |
| AM(37-52)NH₂ | DKDNVAPRSKISPQGY-NH₂ (SEQ ID NO: 4) |
| AM2(1-53)NH₂ | HSGPRRTQAQLLRVGCVLGTCQVQNLSHRLWQLMGPAGRQ |
| | DSAPVDPSSPHSY-NH₂ (SEQ ID NO: 5) |
| AM2(1-47)NH₂ | TQAQLLRVGCVLGTCQVQNLSHRLWQLMGPAGRQDSAPVD |
| | PSSPHSY-NH₂ (SEQ ID NO: 6) |
| AM2(8-47)NH₂ | VGCVLGTCQVQNLSHRLWQLMGPAGRQDSAPVDPSSPHSY- |
| | NH₂ (SEQ ID NO: 7) |
| AM2(17-47)NH₂ | VQNLSHRLWQLMGPAGRQDSAPVDPSSPHSY-NH₂ |
| | (SEQ ID NO: 8) |
| hα-CGRP(1-37)NH₂ | ACDTATCVTHRLAGLLSRSGGVVKNNFVPTNVGSKAF-NH₂ |
| | (SEQ ID NO: 9) |
| α-CGRP(8-37)NH₂ | VTHRLAGLLSRSGGVVKNNFVPTNVGSKAF-NH₂ |
| | (SEQ ID NO: 10) |
| α-CGRP(27-37)NH₂ | FVPTNVGSKAF-NH₂ |
| | (SEQ ID NO: 11) |
| hβ-CGRP(1-37)NH₂ | ACNTATCVTHRLAGLLSRSGGMVKSNFVPTNVGSKAF-NH₂ |
| | (SEQ ID NO: 12) |
| hAMY(1-37)NH₂ | KCNTATCATQRLANFLVHSSNNFGAILSSTNVGSNTY-NH₂ |
| | (SEQ ID NO: 13) |
| hAMY(8-37)NH₂ | ATQRLANFLVHSSNNFGAILSSTNVGSNTY-NH₂ |
| | (SEQ ID NO: 14) |
| hCT(1-32)NH₂ | CGNLSTCMLGTYTQDFNKFHTFPQTAIGVGAP-NH₂ |
| | (SEQ ID NO: 15) |
| hCT(8-32)NH₂ | MLGTYTQDFNKFHTFPQTAIGVGAP-NH₂ |
| | (SEQ ID NO: 16) |
| SCT(1-32)NH₂ | CSNLSTCVLGKLSQELHKLQTYPRTNTGSGTP-NH₂ |
| | (SEQ ID NO: 17) |
| SCT(8-32)NH₂ | VLGKLSQELHKLQTYPRTNTGSGTP-NH₂ (SEQ ID NO: 18) |
| | X₁QNLSHX₇LWQLMGPAGX₁₇QDSAPVDPSSP X₂₉SX₃₁ |
| | (SEQ ID NO: 19) |
| AM2(17-47)NH₂ [H45W] | VQNLSHRLWQLMGPAGRQDSAPVDPSSPWSY-NH₂ |
| | (SEQ ID NO: 20) |
| | X₁X₂X₃LX₅X₆RLWQLMGPAGRDNVAPRX₂₄X₂₅IX₂₇PX₂₉GX₃₁ |
| | (SEQ ID NO: 21) |
| AM2(17-33)-AM(39- | VQNLSHRLWQLMGPAGRDNVAPRSKISPQGY-NH₂ |
| 52)NH₂ | (SEQ ID NO: 22) |
| AM2(17-33)-AM(39- | VQNLSHRLWQLMGPAGRDNVAPRWKISPWGY-NH₂ |
| 52)NH₂ [S45W/Q50W] | (SEQ ID NO: 23) |
| AM2(17-33)-AM(39-52) | VQNLSHRLWQLMGPAGRDNVAPRWKISPWGF-NH₂ |
| NH₂ [S45W/Q50W/Y52F] | (SEQ ID NO: 24) |
| AM2(17-33)-AM(39-52 NH₂ | VQNLSHRLWQLMGPAGRDNVAPRWLISPWGF-NH₂ |
| [S45W/K46L/Q50W/Y52F] | (SEQ ID NO: 25) |
| | X₁X₂X₃X₄LX₆X₇RLWQLMGPAGRDNVAPRX₂₅X₂₆IX₂₈PX₃₀GX₃₂ |
| | (SEQ ID NO: 26) |
| AM(22-28)-AM2(23-33)- | TVQKLAHRLWQLMGPAGRDNVAPRSKISPQGY-NH₂ |
| AM(39-52)NH₂ | (SEQ ID NO: 27) |
| AM(22-28)-AM2(23-33)- | TVQKLAHRLWQLMGPAGRDNVAPRWKISPWGY-NH₂ |
| AM(39-52)NH₂ | (SEQ ID NO: 28) |
| [S45W/Q50W] | |
| AM(22-28)-AM2(23-33)- | TVQKLAHRLWQLMGPAGRDNVAPRWKISPWGF-NH₂ |
| AM(39-52)NH₂ | (SEQ ID NO: 29) |
| [S45W/Q50W/Y52F] | |
| AM(22-28)-AM2(23-33)- | TVQKLAHRLWQLMGPAGRDNVAPRWLISPWGF-NH₂ |
| AM(39-52)NH₂ | (SEQ ID NO: 30) |
| [S45W/K46L/Q50W/Y52F] | |
| | X₁THRLAGRLWQLMGPAGRDNVAPRX₂₅X₂₆IX₂₈PX₃₀GX₃₂ |
| | (SEQ ID NO: 31) |
| CGRP(8-14)-AM2(23-33)- | VTHRLAGRLWQLMGPAGRDNVAPRSKISPQGY-NH₂ |
| AM(39-52)NH₂ | (SEQ ID NO: 32) |
| | X₁TQRLANRLWQLMGPAGRDNVAPRX₂₅X₂₆IX₂₈PX₃₀GX₃₂ |
| | (SEQ ID NO: 33) |
| AMY(8-14)-AM2(23-33)- | ATQRLANRLWQLMGPAGRDNVAPRSKISPQGY-NH₂ |
| AM(39-52)NH₂ | (SEQ ID NO: 34) |
| | X₁LGTYTQRLWQLMGPAGRDNVAPRX₂₅X₂₆IX₂₈PX₃₀GX₃₂ |
| | (SEQ ID NO: 35) |
| hCT(8-14)-AM2(23-33)- | MLGTYTQRLWQLMGPAGRDNVAPRSKISPQGY-NH₂ |
| AM(39-52)NH₂ | (SEQ ID NO: 36) |
| | X₁LGTYTQRLWQLMGPAGRDNVAPRX₂₅X₂₆IX₂₈PX₃₀GX₃₂ |
| | (SEQ ID NO: 37) |
| SCT(8-14)-AM2(23-33)- | VLGTYTQRLWQLMGPAGRDNVAPRSKISPQGY-NH₂ |
| AM(39-52)NH₂ | (SEQ ID NO: 38) |
| | YRQSMNNFQGLRSFGCRFGTCTVQNLSHRLWQLMGPAGRQ |
| | DSAPVDPSSPHSY-NH₂ (SEQ ID NO: 39) |
| | YRQSMNNFQGLRSFGCRFGTCTVQKLAHRLWQLMGPAGRD |
| | NVAPRSKISPQGY-NH₂ (SEQ ID NO: 40) |

In certain non-limiting embodiments, the variant and chimeric peptides of the present disclosure have agonistic activity for at least one receptor complex described herein. In certain other non-limiting embodiments, the variant and chimeric peptides of the present disclosure have antagonistic activity for at least one receptor complex described herein. The variant or chimeric peptide may be bound by linkage, conjugation, complexing, coupling, or otherwise attached to a carrier molecule. The linkage, conjugation, complexing, or coupling may be direct, or indirect via linker molecules known in the art to effect the binding or complexing of a carrier to a peptide.

As noted above, the peptides of the present disclosure can be used to treat a number of conditions and diseases involving the CGRP and AM receptor complexes CLR:RAMP1, CLR:RAMP2, and CLR:RAMP3. Particular examples of cancers that can be treated in a subject by the administration of a peptide of the present disclosure include, but are not limited to: hepatocellular carcinoma, prostate cancer, breast cancer, adrenal tumors, glioblastoma multiforme, melanoma, acute myeloid leukemia, colon cancer, colorectal cancer, renal cancer, lung cancer, osteosarcoma, cervical cancer, ovarian cancer, and pancreatic cancer.

Other particular examples of conditions and diseases that can be treated in a subject by the administration of peptides of the present disclosure include, but are not limited to: sepsis, ulcerative colitis, Crohn's disease, inflammatory bowel disease, myocardial infarction, heart failure, atherosclerotic vascular disease, tissue or organ ischemia, arteriosclerosis obliterans, Buerger's disease, pulmonary hypertension, lymphedema (primary and secondary), promotion of embryo implantation during in vitro fertilization, and preeclampsia. Other examples of conditions and diseases that can be treated in a subject by the administration of peptides of the present disclosure include, but are not limited to: migraine headache, hyperalgesia, menopausal hot flashes, osteoarthritis, rheumatoid arthritis, osteoporosis, pulmonary hypertension, atherosclerosis, myocardial ischemia, gut ischemia, liver ischemia, kidney ischemia, brain ischemia, and ischemic brain injury.

The embodiments of the present disclosure will be more readily understood by reference to the following examples and embodiments, which are included merely for purposes of illustration of certain aspects and embodiments of the inventive concepts and are not intended to be limiting. The following detailed examples and methods describe how to make and use the various variant and chimeric peptides of the present disclosure and are to be construed, as noted above, only as illustrative, and not limitations of the disclosure in any way whatsoever.

### EXPERIMENTAL

### I.

### Methods

### Cell Culture

COS-7 (CRL 1651) and HEK293 (CRL 1573) cells were from American Type Culture Collection (Manassas, VA, USA). Cells were cultured in Dulbecco's modified Eagle's medium (DMEM with 4.5 g/L glucose and L-glutamine) from Lonza (Basel, Switzerland) with 10% v/v fetal bovine serum (Gibco 16000-044). Cells were grown at 37° C., 5% CO₂ in a humidified incubator and passaged twice per week.

### Plasmid Constructs

N-terminally Nanoluciferase (NLuc)-tagged CLR was constructed in the pcDNA3.1(+) backbone using standard PCR and restriction enzyme cloning methods. The fusion construct was inserted between the EcoRI and KpnI sites and included a secretory signal peptide taken from the pHLsec vector followed by NLuc, a short linker, and residues 23-461 of CLR. The RAMP2/3 chimeras and the RAMP3 point mutants were ordered as synthetic GeneArt strings (Thermofisher) using the unoptimized human gene sequences including their natural signal sequences and with addition of EcoRI and XhoI restriction sites. The RAMP2/3 chimeras and RAMP3 mutants were inserted into the pcDNA3.1(+) vector using either restriction enzyme or Gibson assembly methods. All coding sequences were verified by DNA sequencing at the OUHSC laboratory of molecular biology and cytometry core facility. Protein sequences for the coding regions of each of the plasmids are provided in U.S. Provisional Application Ser. No. 63/500,316, filed May 5, 2023. The wild-type CLR and RAMP1-3 expression constructs in the pcDNA3.1(+) vector were from the cDNA resource center. The CAMYEL biosensor plasmid was a kind gift from Drs. Denise Wootten and Patrick Sexton.

### Synthetic Peptides

Synthetic human peptides based on αCGRP(1-37), AM(13-52), and AM2(1-47) were purchased from Bachem (Bubendorf, Switzerland). Custom peptides were synthesized and HPLC-purified by Vivitide (Gardner, MA) or RS Synthesis (Louisville, KY). The lyophilized powders were resuspended at 5-10 mg/ml in sterile ultrapure water. DMSO was included at 9.5% v/v for AM(13-38)-AM2(34-47), 12.5% v/v for AM-TAMRA, and 10% v/v for AM2-TAMRA to improve solubility. Concentrations of the unlabeled peptides were determined by UV absorbance at 280 nm with dilutions in 10 mM Tris-HCl, 1 mM EDTA at pH 8.0. Extinction coefficients were calculated from Tyr, Trp, and cystine content. The concentration of CGRP was determined by the peptide content reported from Bachem. For the TAMIRA-labeled peptides, concentrations were determined by visible absorbance at 560 nm with dilutions in 8M urea, 10 mM Tris-HCL, 1 mM EDTA, pH 8.0 and using the extinction coefficient of TAMIRA (95,000 M⁻¹m⁻¹). Peptides were stored as multiple aliquots at −80° C. to limit the number of freeze-thaws. Tables 2-4 show the amino acid sequences of a non-limiting selection of variant peptides of the present disclosure.

TABLE 2
Additional peptides

| Identifier | Amino acid sequence (SEQ ID NO) |
|---|---|
| CGRP(1-37) | ACDTATCVTHRLAGLLSRSGGVVKNNFVPTNVGSKAF- |
| | NH₂ (SEQ ID NO: 9) |
| AM(13-52) | SFGCRFGTCTVQKLAHQIYQFTDKDKDNVAPRSKISPQ |
| | GY-NH₂ (SEQ ID NO: 2) |
| AM2(1-47) | TQAQLLRVGCVLGTCQVQNLSHRLWQLMGPAGRQDS |
| | APVDPSSPHSY-NH₂ (SEQ ID NO: 6) |
| CGRP(8-37) | VTHRLAGLLSRSGGVVKNNFVPTDVGPWSF-NH₂ |
| N31D/S34P/K35W/A36S] | (SEQ ID NO: 41) |
| AM(22-52) [S48G/Q50W] | TVQKLAHQIYQFTDKDKDNVAPRSKIGPWGY-NH₂ |
| | (SEQ ID NO: 42) |
| AM(13-33)-AM2(28-47) | SFGCRFGTCTVQKLAHQIYQFMGPAGRQDSAPVDPSSP |
| AM-AM2 half | HSY-NH₂ (SEQ ID NO: 43) |
| AM2(8-27)-AM(34-52) | VGCVLGTCQVQNLSHRLWQLTDKDKDNVAPRSKISPQ |
| AM2-AM half | GY-NH₂ (SEQ ID NO: 44) |
| AM(13-38)-AM2(34-47) | SFGCRFGTCTVQKLAHQIYQFTDKDKQDSAPVDPSSPH |
| AM-AM2 ECD | SY-NH₂ (SEQ ID NO: 45) |
| AM2(8-33)-AM(39-52) | VGCVLGTCQVQNLSHRLWQLMGPAGRDNVAPRSKISP |
| AM2-AM | QGY-NH₂ (SEQ ID NO: 46) |
| AM(13-38)-AM2(23-33)-AM(39-52) | SFGCRFGTCTVQKLAHQIYQFTDKDKRLWQLMGPAGR |
| AM-AM2-AM | DNVAPRSKISPQGY-NH₂ (SEQ ID NO: 47) |
| AM2(8-22)-AM(29-38)-AM2(34-47) | VGCVLGTCQVQNLSHQIYQFTDKDKQDSAPVDPSSPHS |
| AM2-AM-AM2 | Y-NH₂ (SEQ ID NO: 48) |
| AM(13-52) N40K-TAMRA | SFGCRFGTCTVQKLAHQIYQFTDKDKD(K-TAMRA) |
| AM-TAMRA | VAPRSKISPQGY-NH₂ (SEQ ID NO: 49) |
| AM2(8-47) D35K-TAMRA | VGCVLGTCQVQNLSHRLWQLMGPAGRQ(K- |
| AM2-TAMRA | TAMRA)SAPVDPSSPHSY-NH₂ (SEQ ID NO: 50) |
| AM(13-52)-NH₂ | SFGCRFGTCTVQKLAHQIYQFTDKDKDNVAPRSKIGPW |
| [S48G, Q50W] | GY-NH₂ (SEQ ID NO: 52) |

TABLE 3
Examples of variants of SEQ ID NO: 21

| Identifier | Amino acid sequence (SEQ ID NO) |
|---|---|
| AM2(17-33)-AM(39-52)-NH₂ | VQNLSHRLWQLMGPAGRDNVAPRSKISPQGY-NH₂ |
| | (SEQ ID NO: 22) |
| AM2(17-33)-AM(39-52)-NH₂ | VQNLSHRLWQLMGPAGRDNVAPRWKISPWGY-NH₂ |
| [S45W/Q50W] | (SEQ ID NO: 23) |
| AM2(17-33)-AM(39-52)-NH₂ | VQNLSHRLWQLMGPAGRDNVAPRSKIGPWGY-NH₂ |
| [S48G/Q50W] | (SEQ ID NO: 57) |
| AM2(17-33)-AM(39-52)-NH₂ | VQNLSHRLWQLMGPAGRDNVAPRWKIGPWGY-NH₂ |
| [S45W/S48G/Q50W] | (SEQ ID NO: 58) |
| AM2(17-33)-AM(39-52)-NH₂ | VQNLSHRLWQLMGPAGRDNVAPRWLISPWGY-NH₂ |
| [S45W/K46L/Q50W] | (SEQ ID NO: 59) |
| AM2(17-33)-AM(39-52)-NH₂ | VQNLSHRLWQLMGPAGRDNVAPRWRISPWGY-NH₂ |
| S45W/K46R/Q50W | (SEQ ID NO: 60) |
| AM2(17-33)-AM(39-52)-NH₂ | VQNLSHRLWQLMGPAGRDNVAPRSLIGPWGY-NH₂ |
| [K46L/S48G/Q50W] | (SEQ ID NO: 61) |
| AM2(17-33)-AM(39-52)-NH₂ | VQNLSHRLWQLMGPAGRDNVAPRSRIGPWGY-NH₂ |
| K46R/S48G/Q50W] | (SEQ ID NO: 62) |
| AM2(17-33)-AM(39-52)-NH₂ | VQNLSHRLWQLMGPAGRDNVAPRWKISPWGF-NH₂ |
| [S45W/Q50W/Y52F] | (SEQ ID NO: 24) |
| AM2(17-33)-AM(39-52)-NH₂ | VQNLSHRLWQLMGPAGRDNVAPRSKIGPWGF-NH₂ |
| [S48G/Q50W/Y52F] | (SEQ ID NO: 63) |
| AM2(17-33)-AM(39-52)-NH₂ | VQNLSHRLWQLMGPAGRDNVAPRWKIGPWGF-NH₂ |
| [S45W/S48G/Q50W/Y52F] | (SEQ ID NO: 64) |
| AM2(17-33)-AM(39-52)-NH₂ | VQNLSHRLWQLMGPAGRDNVAPRWLIGPWGY-NH₂ |
| [S45W/K46L/S48G/Q50W] | (SEQ ID NO: 65) |
| AM2(17-33)-AM(39-52)-NH₂ | VQNLSHRLWQLMGPAGRDNVAPRWRIGPWGY-NH₂ |
| [S45W/K46R/S48G/Q50W] | (SEQ ID NO: 66) |
| AM2(17-33)-AM(39-52)-NH₂ | VQNLSHRLWQLMGPAGRDNVAPRSLIGPWGF-NH₂ |
| [K46L/S48G/Q50W/Y52F] | (SEQ ID NO: 67) |
| AM2(17-33)-AM(39-52)-NH₂ | VQNLSHRLWQLMGPAGRDNVAPRSRIGPWGF-NH₂ |
| [K46R/S48G/Q50W/Y52F] | (SEQ ID NO: 68) |
| AM2(17-33)-AM(39-52)-NH₂ | VQNLSHRLWQLMGPAGRDNVAPRWRISPWGF-NH₂ |
| S45W/K46L/Q50W/Y52F] | (SEQ ID NO: 25) |
| AM2(17-33)-AM(39-52)-NH₂ | VQNLSHRLWQLMGPAGRDNVAPRWRISPWGF-NH₂ |
| [S45W/K46R/Q50W/Y52F] | (SEQ ID NO: 69) |
| AM2(17-33)-AM(39-52)-NH₂ | VQNLSHRLWQLMGPAGRDNVAPRWLIGPWGF-NH₂ |
| [S45W/K46L/S48G/Q50W/Y52F] | (SEQ ID NO: 70) |
| AM2(17-33)-AM(39-52)-NH₂ | VQNLSHRLWQLMGPAGRDNVAPRWRIGPWGF-NH₂ |
| [S45W/K46R/S48G/Q50W/Y52F] | (SEQ ID NO: 71) |

TABLE 4
Examples of variants of SEQ ID NO: 26

| Identifier | Amino acid sequence (SEQ ID NO) |
|---|---|
| AM(22-28)-AM2(23-33)-AM(39-52)- | TVQKLAHRLWQLMGPAGRDNVAPRSKISPQGY-NH₂ |
| NH₂ | (SEQ ID NO: 27) |
| AM(22-28)-AM2(23-33)-AM(39-52)- | TVQKLAHRLWQLMGPAGRDNVAPRWKISPWGY-NH₂ |
| NH₂ [S45W/Q50W] | (SEQ ID NO: 28) |
| AM(22-28)-AM2(23-33)-AM(39-52)- | TVQKLAHRLWQLMGPAGRDNVAPRSKIGPWGY-NH₂ |
| NH₂ [S48G/Q50W] | (SEQ ID NO: 72) |
| AM(22-28)-AM2(23-33)-AM(39-52)- | TVQKLAHRLWQLMGPAGRDNVAPRSKIGPWGF-NH₂ |
| NH₂ [S48G/Q50W/Y52F] | (SEQ ID NO: 73) |
| AM(22-28)-AM2(23-33)-AM(39-52)- | TVQKLAHRLWQLMGPAGRDNVAPRWLISPWGY-NH₂ |
| NH₂ [S45W/K46L/Q50W] | (SEQ ID NO: 74) |
| AM(22-28)-AM2(23-33)-AM(39-52)- | TVQKLAHRLWQLMGPAGRDNVAPRWRISPWGY-NH₂ |
| NH₂ [S45W/K46R/Q50W] | (SEQ ID NO: 75) |
| AM(22-28)-AM2(23-33)-AM(39-52)- | TVQKLAHRLWQLMGPAGRDNVAPRWKIGPWGY-NH₂ |
| NH₂ [S45W/S48G/Q50W] | (SEQ ID NO: 76) |
| AM(22-28)-AM2(23-33)-AM(39-52)- | TVQKLAHRLWQLMGPAGRDNVAPRWKISPWGF-NH₂ |
| NH₂ [S45W/Q50W/Y52F] | (SEQ ID NO: 29) |
| AM(22-28)-AM2(23-33)-AM(39-52)- | TVQKLAHRLWQLMGPAGRDNVAPRSLIGPWGY-NH₂ |
| NH₂ [K46L/S48G/Q50W] | (SEQ ID NO: 77) |
| AM(22-28)-AM2(23-33)-AM(39-52)- | TVQKLAHRLWQLMGPAGRDNVAPRSRIGPWGY-NH₂ |
| NH₂ [K46R/S48G/Q50W] | (SEQ ID NO: 78) |
| AM(22-28)-AM2(23-33)-AM(39-52)- | TVQKLAHRLWQLMGPAGRDNVAPRWLIGPWGY-NH₂ |
| NH₂ [S45W/ K46L/S48G/Q50W] | (SEQ ID NO: 79) |
| AM(22-28)-AM2(23-33)-AM(39-52)- | TVQKLAHRLWQLMGPAGRDNVAPRWRIGPWGY-NH₂ |
| NH₂ [S45W/ K46R/S48G/Q50W] | (SEQ ID NO: 80) |
| AM(22-28)-AM2(23-33)-AM(39-52)- | TVQKLAHRLWQLMGPAGRDNVAPRSLIGPWGF-NH₂ |
| NH₂ [K46L/S48G/Q50W/Y52F] | (SEQ ID NO: 81) |
| AM(22-28)-AM2(23-33)-AM(39-52)- | TVQKLAHRLWQLMGPAGRDNVAPRSRIGPWGF-NH₂ |
| NH₂ [K46R/S48G/Q50W/Y52F] | (SEQ ID NO: 82) |
| AM(22-28)-AM2(23-33)-AM(39-52)- | TVQKLAHRLWQLMGPAGRDNVAPRWKIGPWGF-NH₂ |
| NH₂ [S45W/S48G/Q50W/Y52F] | (SEQ ID NO: 83) |
| AM(22-28)-AM2(23-33)-AM(39-52) | TVQKLAHRLWQLMGPAGRDNVAPRWRISPWGF-NH₂ |
| [S45W/K46L/Q50W/Y52F] | (SEQ ID NO: 30) |
| AM(22-28)-AM2(23-33)-AM(39-52)- | TVQKLAHRLWQLMGPAGRDNVAPRWRISPWGF-NH₂ |
| NH₂ [S45W/K46R/Q50W/Y52F] | (SEQ ID NO: 84) |
| AM(22-28)-AM2(23-33)-AM(39-52)- | TVQKLAHRLWQLMGPAGRDNVAPRWLIGPWGF-NH₂ |
| NH₂ [S45W/K46L/S48G/Q50W/Y52F] | (SEQ ID NO: 85) |
| AM(22-28)-AM2(23-33)-AM(39-52)- | TVQKLAHRLWQLMGPAGRDNVAPRWRIGPWGF-NH₂ |
| NH₂ [S45W/K46R/S48G/Q50W/Y52F] | (SEQ ID NO: 86) |

TABLE 5
Variants of peptide AM(13-28)-AM2(23-33)-AM(39-52)

| Identifier | Amino acid sequence (SEQ ID NO) |
|---|---|
| AM(13-28)-AM2(23-33)-AM(39-52)- | SFGCRFGTCTVQKLAHRLWQLMGPAGRDNVAPRSKIG |
| NH₂ [S48G/Q50W] | PWGY-NH₂ (SEQ ID NO: 51) |
| AM(13-28)-AM2(23-33)-AM(39-52)- | SFGCRFGTCTVQKLAHRLWQLMGPAGRDNVAPRWKIS |
| NH₂ [S45W/Q50W | PWGY-NH₂ (SEQ ID NO: 87) |
| AM(13-28)-AM2(23-33)-AM(39-52)- | SFGCRFGTCTVQKLAHRLWQLMGPAGRDNVAPRSKIG |
| NH₂ [S48G/Q50W/Y52F] | PWGF-NH₂ (SEQ ID NO: 88) |
| AM(13-28)-AM2(23-33)-AM(39-52)- | SFGCRFGTCTVQKLAHRLWQLMGPAGRDNVAPRWLIS |
| NH₂ [S45W/K46L/Q50W] | PWGY-NH₂ (SEQ ID NO: 89) |
| AM(13-28)-AM2(23-33)-AM(39-52)- | SFGCRFGTCTVQKLAHRLWQLMGPAGRDNVAPRWRIS |
| NH₂ [S45W/K46R/Q50W] | PWGY-NH₂ (SEQ ID NO: 90) |
| AM(13-28)-AM2(23-33)-AM(39-52)- | SFGCRFGTCTVQKLAHRLWQLMGPAGRDNVAPRWKIG |
| NH₂ [S45W/S48G/Q50W] | PWGY-NH₂ (SEQ ID NO: 91) |
| AM(13-28)-AM2(23-33)-AM(39-52)- | SFGCRFGTCTVQKLAHRLWQLMGPAGRDNVAPRWKIS |
| NH₂ [S45W/Q50W/Y52F] | PWGF-NH₂ (SEQ ID NO: 92) |
| AM(13-28)-AM2(23-33)-AM(39-52)- | SFGCRFGTCTVQKLAHRLWQLMGPAGRDNVAPRSLIG |
| NH₂ [K46L/S48G/Q50W] | PWGY-NH₂ (SEQ ID NO: 93) |
| AM(13-28)-AM2(23-33)-AM(39-52)- | SFGCRFGTCTVQKLAHRLWQLMGPAGRDNVAPRSRIG |
| NH₂ [K46R/S48G/Q50W] | PWGY-NH₂ (SEQ ID NO: 94) |
| AM(13-28)-AM2(23-33)-AM(39-52)- | SFGCRFGTCTVQKLAHRLWQLMGPAGRDNVAPRWLIG |
| NH₂ [S45W/K46L/S48G/Q50W] | PWGY-NH₂ (SEQ ID NO: 95) |
| AM(13-28)-AM2(23-33)-AM(39-52)- | SFGCRFGTCTVQKLAHRLWQLMGPAGRDNVAPRWRIG |
| NH₂ [S45W/K46R/S48G/Q50W] | PWGY-NH₂ (SEQ ID NO: 96) |
| AM(13-28)-AM2(23-33)-AM(39-52)- | SFGCRFGTCTVQKLAHRLWQLMGPAGRDNVAPRSLIG |
| NH₂ [K46L/S48G/Q50W/Y52F] | PWGF-NH₂ (SEQ ID NO: 97) |
| AM(13-28)-AM2(23-33)-AM(39-52)- | SFGCRFGTCTVQKLAHRLWQLMGPAGRDNVAPRSRIG |
| NH₂ [K46R/S48G/Q50W/Y52F] | PWGF-NH₂ (SEQ ID NO: 98) |
| AM(13-28)-AM2(23-33)-AM(39-52)- | SFGCRFGTCTVQKLAHRLWQLMGPAGRDNVAPRWKIG |
| NH₂ [S45W/S48G/Q50W/Y52F | PWGF-NH₂ (SEQ ID NO: 99) |
| AM(13-28)-AM2(23-33)-AM(39-52)- | SFGCRFGTCTVQKLAHRLWQLMGPAGRDNVAPRWRIS |
| NH₂ [S45W/K46L/Q50W/Y52F] | PWGF-NH₂ (SEQ ID NO: 100) |
| AM(13-28)-AM2(23-33)-AM(39-52)- | SFGCRFGTCTVQKLAHRLWQLMGPAGRDNVAPRWRIS |
| NH₂ [S45W/K46R/Q50W/Y52F] | PWGF-NH₂ (SEQ ID NO: 101) |
| AM(13-28)-AM2(23-33)-AM(39-52) | SFGCRFGTCTVQKLAHRLWQLMGPAGRDNVAPRWLIG |
| NH₂ [S45W/K46L/S48G/Q50W/Y52F] | PWGF-NH₂ (SEQ ID NO: 102) |
| AM(13-28)-AM2(23-33)-AM(39-52)- | SFGCRFGTCTVQKLAHRLWQLMGPAGRDNVAPRWRIG |
| NH₂ [S45W/K46R/S48G/Q50W/Y52F] | PWGF-NH₂ (SEQ ID NO: 103) |

TABLE 6
Variants of peptide AM2(8-33) - AM(39-52)

| Identifier | Amino acid sequence (SEQ ID NO) |
|---|---|
| AM2(8-33)-AM(39-52)-NH₂ | VGCVLGTCQVQNLSHRLWQLMGPAGRDNVAPRSKIGP |
| [S48G/Q50W] | WGY-NH₂ (SEQ ID NO: 53) |
| AM2(8-33)-AM(39-52)-NH₂ | VGCVLGTCQVQNLSHRLWQLMGPAGRDNVAPRWKIS |
| [S45W/Q50W] | PWGY-NH₂ (SEQ ID NO: 104) |
| AM2(8-33)-AM(39-52)-NH₂ | VGCVLGTCQVQNLSHRLWQLMGPAGRDNVAPRWKIG |
| [S45W/S48G/Q50W] | PWGY-NH₂ (SEQ ID NO: 105) |
| AM2(8-33)-AM(39-52)-NH₂ | VGCVLGTCQVQNLSHRLWQLMGPAGRDNVAPRWLISP |
| [S45W/K46L/Q50W] | WGY-NH₂ (SEQ ID NO: 106) |
| AM2(8-33)-AM(39-52)-NH₂ | VGCVLGTCQVQNLSHRLWQLMGPAGRDNVAPRWRISP |
| [S45W/K46R/Q50W] | WGY-NH₂ (SEQ ID NO: 107) |
| AM2(8-33)-AM(39-52)-NH₂ | VGCVLGTCQVQNLSHRLWQLMGPAGRDNVAPRSLIGP |
| [K46L/S48G/Q50W] | WGY-NH₂ (SEQ ID NO: 108) |
| AM2(8-33)-AM(39-52)-NH₂ | VGCVLGTCQVQNLSHRLWQLMGPAGRDNVAPRSRIGP |
| K46R/S48G/Q50W] | WGY-NH₂ (SEQ ID NO: 109) |
| AM2(8-33)-AM(39-52)-NH₂ | VGCVLGTCQVQNLSHRLWQLMGPAGRDNVAPRWKIS |
| S45W/Q50W/Y52F] | PWGF-NH₂ (SEQ ID NO: 110) |
| AM2(8-33)-AM(39-52)-NH₂ | VGCVLGTCQVQNLSHRLWQLMGPAGRDNVAPRSKIGP |
| S48G/Q50W/Y52F | WGF-NH₂ (SEQ ID NO: 111) |
| AM2(8-33)-AM(39-52)-NH₂ | VGCVLGTCQVQNLSHRLWQLMGPAGRDNVAPRWKIG |
| [S45W/S48G/Q50W/Y52F] | PWGF-NH₂ (SEQ ID NO: 112) |
| AM2(8-33)-AM(39-52)-NH₂ | VGCVLGTCQVQNLSHRLWQLMGPAGRDNVAPRWLIG |
| [S45W/K46L/S48G/Q50W] | PWGY-NH₂ (SEQ ID NO: 113) |
| AM2(8-33)-AM(39-52)-NH₂ | VGCVLGTCQVQNLSHRLWQLMGPAGRDNVAPRWRIG |
| [S45W/K46R/S48G/Q50W] | PWGY-NH₂ (SEQ ID NO: 114) |
| AM2(8-33)-AM(39-52)-NH₂ | VGCVLGTCQVQNLSHRLWQLMGPAGRDNVAPRSLIGP |
| [K46L/S48G/Q50W/Y52F | WGF-NH₂ (SEQ ID NO: 115) |
| AM2(8-33)-AM(39-52)-NH₂ | VGCVLGTCQVQNLSHRLWQLMGPAGRDNVAPRSRIGP |
| [K46R/S48G/Q50W/Y52F] | WGF-NH₂ (SEQ ID NO: 116) |
| AM2(8-33)-AM(39-52)-NH₂ | VGCVLGTCQVQNLSHRLWQLMGPAGRDNVAPRWRISP |
| [S45W/K46L/Q50W/Y52F] | WGF-NH₂ (SEQ ID NO: 117) |
| AM2(8-33)-AM(39-52)-NH₂ | VGCVLGTCQVQNLSHRLWQLMGPAGRDNVAPRWRISP |
| [S45W/K46R/Q50W/Y52F] | WGF-NH₂ (SEQ ID NO: 118) |
| AM2(8-33)-AM(39-52)-NH₂ | VGCVLGTCQVQNLSHRLWQLMGPAGRDNVAPRWLIG |
| [S45W/K46L/S48G/Q50W/Y52F] | PWGF-NH₂ (SEQ ID NO: 119) |
| AM2(8-33)-AM(39-52)-NH₂ | VGCVLGTCQVQNLSHRLWQLMGPAGRDNVAPRWRIG |
| [S45W/K46R/S48G/Q50W/Y52F] | PWGF-NH₂ (SEQ ID NO: 56) |

Real-Time BRET cAMP Biosensor Assay

COS-7 or HEK293 cells were seeded at 20,000 cells/well and 100 μL/well in a 96-well white plate and incubated for 24 hrs. The cells were transiently transfected with 250 ng total DNA and 375 ng of BPEI per well. For COS-7, 25 ng receptor, 25 ng RAMP, 125 ng CAMYEL biosensor, and 75 ng empty pcDNA3.1(+) was used. For HEK293, 10 ng receptor, 10 ng RAMP, 175 ng CAMYEL biosensor, and 55 ng empty vector was used. Two days after transfection the cells were washed with PBS and incubated for 30 min at room temperature in sterile filtered 25 mM NaHEPES pH 7.4, 104 mM NaCl, 5 mM KCl, 1 mM KH₂PO₄, 1.2 mM MgSO₄, 2 mM CaCl₂, 1 mg/mL fatty-acid-free bovine serum albumin (FAF-BSA), and 5 mM glucose. Coelenterazine h was added at 10 μM and incubated for 5 min at room temperature. The emissions at 475 and 535 nm were read in a PolarSTAR Omega plate reader (BMG Labtech, Ortenberg, Germany) using a dual luminescence optic for 5 min to establish the baseline. Agonist was manually added to 100 nM with reading for 15 min followed by manual addition of antagonist (10 μM) or forskolin (10 μM) or buffer with an additional 40 min reading. The antagonists used were αCGRP(8-37)[N31D/S34P/K35W/A36S] (SEQ ID NO:41) for CLR-RAMP1 and AM(22-52)[S48G/Q50W] (SEQ ID NO:42) for CLR-RAMP2/3. For assays performed at 37° C., the cells were incubated in a 37° C. incubator, and the plate reader was heated to 37° C. The 475/535 BRET ratio was used to plot the data over time with buffer control subtracted from each curve. The decay phase after antagonist addition was fit to a one-phase exponential decay model in GraphPad Prism v9.4.1 (GraphPad Software).

LANCE cAMP Accumulation Assay

cAMP accumulation assays were performed. In brief, COS-7 cells were seeded in a 96-well plate on day one. Cells were transiently transfected with receptors on day two and on day four the cells were stimulated with agonist for 15 min at 37° C. in the presence of IBMX. The cells were lysed and the cAMP was measured using a LANCE ultra cAMP Detection Kit (Perkin Elmer) according to the manufacturer's instructions.

### NLuc-CLR-RAMP3 Membranes Preparation

COS-7 cells were seeded at 2.5 million cells/dish into ten 150 mm² plastic culture dishes and grown for 2 days. The cells were transiently transfected with 50 μg DNA (2 μg NLuc-CLR, 2 μg RAMP3, and 46 μg empty pcDNA3.1) and 75 μg PEI/dish. After two days the media was aspirated and the cells were washed with PBS and harvested with ice-cold PBS and 5 mM EDTA with a cell scraper and pelleted in a centrifuge at 1000×g for 5 min at 4° C. All remaining steps were on ice or at 4° C. The pellets were resuspended in hypotonic buffer (25 mM NaHEPES pH 7.5, 2 mM MgCl₂, 1 mM EDTA with 1×EDTA-free PIERCE protease inhibitor tablet (PI)). The cells were homogenized with an Ultra Turrax for 30 s at 10k rpm followed by a 10 min incubation. Cell debris was pelleted at 800×g for 10 min. The supernatant was transferred to ultracentrifuge tubes and centrifuged at 100k×g for 1 hr. The cell pellets were resuspended in wash buffer (25 mM NaHEPES pH 7.5, 250 mM NaCl, 2 mM MgCl₂, 1 mM EDTA, and 1×PI). The membranes were homogenized and spun in the ultracentrifuge as before. The membrane pellets were combined in storage buffer (25 mM NaHEPES pH 7.5, 25 mM NaCl, 2 mM MgCl₂, 10% v/v glycerol, and 1×PI), homogenized, aliquoted, and flash-frozen in liquid nitrogen for storage at −80° C. The protein concentration was determined using the DC protein assay (BioRad) per the manufacturer's instructions.

### NanoBRET™ Ligand Binding Assays

Each binding assay format used NLuc-CLR-RAMP3 membranes at a concentration of 0.0075 mg/mL in a binding buffer of 25 mM NaHEPES pH 7.4, 104 mM NaCl, 5 mM KCl, 1 mM KH₂PO₄, 3 mM MgSO₄, 2 mM CaCl₂, 1 mg/mL FAF-BSA, 50 μM GTPTS, and 50 μg/mL saponin. The equilibrium assays were performed at room temperature and the kinetic assays were at 25° C. For equilibrium binding, 3-fold serial dilutions of AM-TAMRA and AM2-TAMRA were incubated with the membranes for 3 hr at room temperature in a 96-well white plate. Furimazine substrate (Promega) was added at 1× and incubated for 5 min. Emission was read at 460 and 610LP nm in the PolarSTAR Omega plate reader. Agonist concentration was plotted against the BRET ratio of 610/460 and fit to a one site specific binding model in GraphPad Prism after subtracting the buffer control.

For association kinetics, 2-fold serial dilutions of AM-TAMRA and AM2-TAMRA in binding buffer were added to a 96 well white plate at 2×. The membranes at 2× were incubated for 15 min in binding buffer followed by addition of furimazine at 2× with a further 5 min incubation and then loaded into the plate reader injector. The membranes were injected into the peptide serial dilutions in the plate with reading every 16 s for 40 min. The BRET ratio 610/460 was plotted against time with background subtraction and each curve was fit to a one-phase association exponential model in GraphPad Prism using the first 10 min of data to minimize the effects of signal decay.

For dissociation kinetics, the membranes and AM-TAMRA or AM2-TAMRA (10 nM) in binding buffer were incubated in a 96-well white plate for 15 min followed by furimazine addition at 1× for 5 min. Emission at 460 and 610 nm were read every 13 sec for 5 min to establish a baseline prior to injection of buffer or antagonist to initiate dissociation. Binding buffer or AM(22-52)[S48G/Q50W] (SEQ ID NO:42) antagonist (1 μM) loaded into the injectors were injected into the plate, which was read for 1 hr. The BRET ratio 610/460 was plotted against time with background subtraction. The curves were normalized to their corresponding buffer control injections as 100% to account for the signal decay with time. The normalized dissociation curves were fit to a two-phase exponential decay model in GraphPad Prism constraining the AM-TAMRA and AM2-TAMRA curves to have the same plateau.

### Native PAGE Assays for Agonist-GPCR-miniGs Coupling and Thermostability

The coupling assays were performed up to the point of formation of quaternary agonist-CLR-RAMP3-miniGs complexes, and they used the previous MBP-CLR-EGFP:MBP-RAMP3 membrane preparation and H6-sumo-miniGs freshly purified. Three-fold serial dilutions of AM(22-52)[S48G/Q50W] (SEQ ID NO:42) antagonist were added to the pre-formed quaternary complexes and incubated for an additional 2 or 19 hours at 4° C. on a rocking shaker. The samples were centrifuged, and the supernatants were analyzed by native PAGE with EGFP fluorescence imaging. The thermostability assays were performed using LMNG/CHS, except with the inclusion of ligands and using finer temperature increments.

### Rebuilding and Refinement of Cryo-EM Structures in Preparation for MD

AlphaFold2 was used via Colabfold to predict the structures of the AM- and AM2-bound CLR-RAMP3 complexes. Using Pymol (Schrodinger, LLC) and/or Coot, the receptor ECD complexes from the AlphaFold models were extracted and the peptides were modeled with the AM and AM2 conformations from high-resolution crystal structures of their complexes with CLR-RAMP2 ECD (4RWF) and CLR-RAMP1 ECD (6D1U), respectively. These peptide-bound CLR-RAMP3 ECD complex models were used to replace the corresponding complexes in 6UUS (AM) and 6UVA (AM2) as starting points for rebuilding. The models were manually rebuilt in Coot guided by fitting to the multiple cryo-EM density maps deposited in the EMDB for the AM (20901) and AM2 (20906) structures. The “combine focused maps” tool in Phenix was used to generate a single best composite map from the multiple maps for each structure and the rebuilt models were refined to their respective composite maps using Phenix real-space refinement.

### MD Simulations

The rebuilt structures were prepared for MD simulation by rebuilding two missing CLR loops with MODELLER: 351-362 and 323-329. The membrane-solvated system was then constructed in CHARMM-GUI, retaining the two resolved cholesterol molecules. In the CHARMM-GUI system preparation tool, GlcNAc glycosylations were added to N66, N118 and N123 of CLR, as well as to N29, N58, N71 and N103 of RAMP3. The C-terminal residue of each peptide was amidated using the CT2 patch. The membranes were constructed using a roughly 3:2 ratio of POPC:cholesterol, with a 10 Å clearance of the protein towards the edge of the box in the x and y dimensions. This resulted in 27 cholesterol and 39 POPC molecules in the lower (intracellular) leaflet and 26 cholesterol and 39 POPC molecules in the upper (extracellular) leaflet for the AM-AM₂R system. The AM2-AM₂R system was slightly smaller in the x and y dimensions, resulting in 26 cholesterol and 36 POPC in the lower leaflet and 24 cholesterol and 36 POPC in the upper leaflet.

The systems were solvated using a 10 Å clearance with TIP3 water molecules, resulting in 16029 and 15559 waters for the AM-AM₂R and AM2-AM₂R systems, respectively. NaCl was added to each system at a concentration of 0.15 M. The equilibration and heating steps were run as specified in the scripts provided by CHARMM-GUI, with the addition of an auxiliary restraint force acting on all non-hydrogen CLR atoms with a z-coordinate greater than 9.5 nm. This force was meant to mimic the stabilizing effect of the G protein, even though it was omitted from the simulation to minimize the computational cost. The restraint force was implemented using a CustomExternalForce function in OpenMM, that restrained the positions of the atoms to their original positions using a harmonic restraint energy with a force constant of 5000 kJ/mol/nm². This force acted on the entire C-terminal helix of CLR (residues 389 to 402), as well as residues from the other three intracellular loops (residues 165-172, 240-251, 318-328). The equilibration included 5000 steps of energy minimization followed by a six-step minimization protocol (125000 or 250000 steps each, 1 fs or 2 fs timestep), where positional restraints on the protein backbone, protein side-chain and lipids, were gradually reduced. Dihedral restraints on lipids and carbohydrates were similarly reduced over the course of the equilibration. An OpenMM MonteCarloMembraneBarostat function was employed, with a pressure coupling frequency of 100 steps and a reference pressure of 1 bar. Following equilibration, all restraints besides the G protein CLR restraints were removed and the system was allowed to relax for 10 ns at a 2 fs timestep.

The final frames of each system were used as initial structures for a set of weighted ensemble simulations with the REVO (Resampling Ensembles by Variation Optimization) method using the wepy software. Three independent replicates, with 48 trajectories each, were used for each system. Each ensemble was run for 2000 cycles of resampling, with 20 ps of dynamics in each cycle. The combined sampling time across all replicates was 5.76 μs per system, or 11.52 μs in total. The resampling procedure following each cycle used the REVO resampling function, with a minimum probability of 10⁻¹², a maximum probability of 0.1 and a weight-based novelty function with a distance exponent of 4. The distance metric used for the trajectory variation function was the RMSD of the peptide following alignment to the entire CLR protein. This distance is calculated between the trajectories in the ensemble and is used to identify “outliers” that are preferentially chosen for cloning, as well as quasi-redundant trajectories that are preferentially chosen for merging. Merging operations could only be performed for trajectories that were within 8 Å of each other, according to the distance metric described above. The vast majority of the trajectory pairs met this criterion.

All probability-based analyses of these datasets, such as the RMSD probability distributions and distance probability distributions, employed the weights of the trajectories. Water density isosurfaces were computed using the volmap tool of VMD.

### Statistical Analysis

For all assays, duplicate technical replicates were used, except for the native PAGE assays, and reported as mean±SD in the representative plots. All experiments were done with three independent replicates on different days and reported as mean±SEM. Statistical analysis was performed using GraphPad Prism on the log form of the values. One-way ANOVA with the Tukey's post hoc test was used at a confidence interval of 99.9% reaching a statistical significance of p<0.001, comparing the mean of the three independent replicates. Only the most important comparisons that reached statistical significance were shown in figures.

### Results

AM2 Exhibits Long-Duration cAMP Signaling at the AM₂R (CLR-RAMP3)

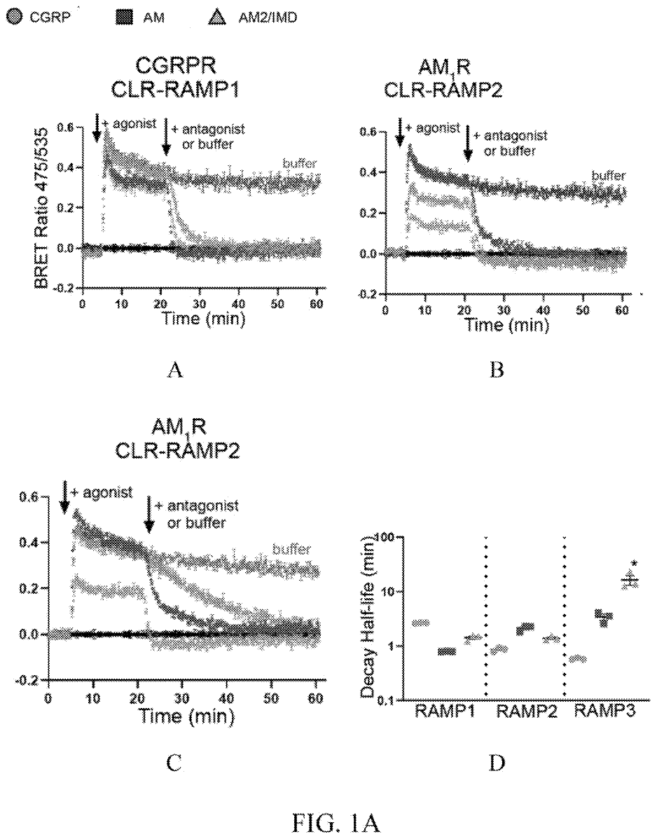

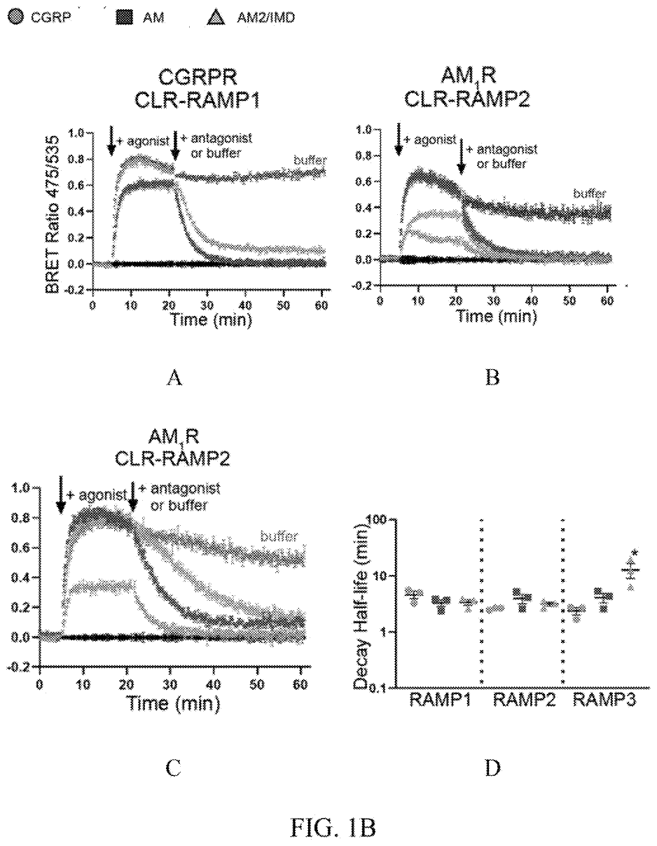

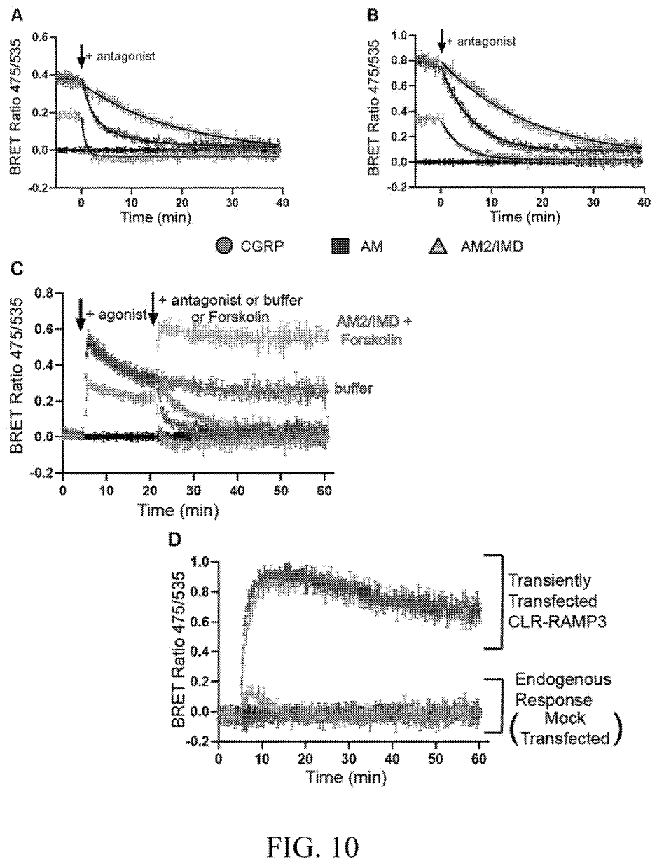

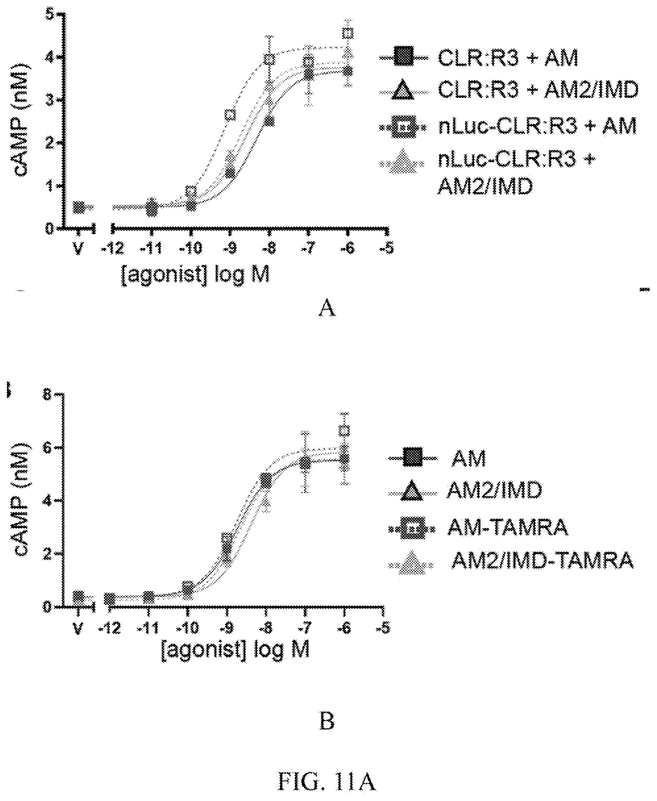

We measured cAMP in real-time upon activation of each CLR-RAMP complex transiently expressed in COS-7 cells using the BRET cAMP biosensor CAMYEL. COS-7 cells were used because they lack endogenous expression of CLR and RAMP accessory proteins. The cells were stimulated at RT with 100 nM CGRP, AM, or AM2 for 15 min followed by challenge with recently developed high affinity CGRP or AM variant antagonist peptides (10 μM) (J. M. Booe, M. L. Warner, A. A. Pioszak, Picomolar Affinity Antagonist and Sustained Signaling Agonist Peptide Ligands for the Adrenomedullin and Calcitonin Gene-Related Peptide Receptors. ACS pharmacology & translational science 3, 759-772 (2020)). The signal decay after antagonist addition provided a measure of signal duration and we reasoned that it would also act as a proxy for agonist dissociation from the receptor. Each agonist gave a rise and fall to steady-state curve at each receptor in the absence of antagonist, and the BRET signal levels reflected the expected agonist potency rank orders (FIG. 1A(A-C)). Upon antagonist challenge, the cAMP BRET signal decayed relatively quickly to baseline for each agonist at the CLR-RAMP1 and CLR-RAMP2 complexes and for CGRP and AM at the CLR-RAMP3 complex ((FIG. 1A(A-C)). In stark contrast, the cAMP signal observed for AM2 at the CLR-RAMP3 complex decayed substantially slower (FIG. 1A(C)). The decay phase of each curve was best fit by a one-phase exponential decay model (FIG. 10(A,B). The decay rates, half-lives, and time constants are summarized in Table 7 and the half-lives are shown in a scatter plot (FIG. 1B(A)). The half-lives for most of the agonist-receptor combinations were within 0.6-3.5 min, whereas AM2 at AM₂R exhibited a decay half-life of ~17 min (FIG. 1B(A)). A slower decay rate for AM2-AM₂R was also observed in experiments performed at the physiological temperature of 37° C. (FIG. 10(C)).

We extended these experiments to the receptors transiently expressed in HEK293 cells to test the robustness of the finding. The HEK293 cells exhibited a small endogenous response to CGRP in the absence of transfected receptors, but this was not large enough to confound the results with transfected receptors (FIG. 10(D)). No endogenous response was observed upon AM or AM2 stimulation. The cAMP signaling kinetic profiles in HEK293 cells were similar to those observed in COS-7 cells ((FIG. 1B(A-D)). Importantly, the slower decay of the AM2 signal at AM₂R was reproduced in the second cell line (t_{1/2}~13 min) (Table 7). The cAMP signaling kinetics results indicated that the AM2-AM₂R pairing is unique in exhibiting a long-duration cAMP signaling capability. AM2 is a slow off-rate, long residence time ligand of the AM₂R.

TABLE 7
Summary of cAMP signaling decay kinetics for CGRP, AM, and
AM2 in COS-7 and HEK293 cells at each receptor heterodimer

| | COS-7 | | | HEK293 | | |
|---|---|---|---|---|---|---|
| | Observed | Time | | Observed | Time | |
| | decay rate | Constant τ | Half-life | decay rate | Constant τ | Half-life |
| | (min⁻¹ ± SEM) | (min ± SEM)* | (min ± SEM)^{†} | (min⁻¹ ± SEM) | (min ± SEM) | (min ± SEM) |
| CLR-RAMP1 | | | | | | |
| CGRP | 0.26 ± 0.0024 | 3.80 ± 0.035 | 2.6 ± 0.024 | 0.16 ± 0.028 | 6.7 ± 1.0 | 4.6 ± 0.70 |
| AM | 0.87 ± 0.0055 | 1.2 ± 0.0073 | 0.80 ± 0.0051 | 0.22 ± 0.035 | 4.8 ± 0.67 | 3.3 ± 0.46 |
| AM2 | 0.49 ± 0.038 | 2.1 ± 0.15 | 1.4 ± 0.10 | 0.21 ± 0.027 | 4.9 ± 0.57 | 3.4 ± 0.39 |
| CLR-RAMP2 | | | | | | |
| CGRP | 0.80 ± 0.042 | 1.3 ± 0.065 | 0.87 ± 0.045 | 0.27 ± 0.0077 | 3.8 ± 0.11 | 2.6 ± 0.073 |
| AM | 0.33 ± 0.025 | 3.1 ± 0.22 | 2.1 ± 0.15 | 0.19 ± 0.040 | 5.8 ± 1.1 | 4.0 ± 0.76 |
| AM2 | 0.50 ± 0.025 | 2.0 ± 0.11 | 1.4 ± 0.074 | 0.22 ± 0.018 | 4.6 ± 0.37 | 3.2 ± 0.25 |
| CLR-RAMP3 | | | | | | |
| CGRP | 1.2 ± 0.029 | 0.85 ± 0.022 | 0.59 ± 0.015 | 0.31 ± 0.053 | 3.5 ± 0.51 | 2.4 ± 0.35 |
| AM | 0.21 ± 0.031 | 4.9 ± 0.67 | 3.4 ± 0.46 | 0.19 ± 0.042 | 5.9 ± 1.2 | 4.1 ± 0.80 |
| AM2 | 0.045 ± 0.0075 | 24 ± 4.7 | 17 ± 3.3 | 0.066 ± 0.022 | 19 ± 5.5 | 13 ± 3.8 |

*Time constant calculated as the inverse of decay rate
*Half-life calculated as ln 2 divided by the decay rate

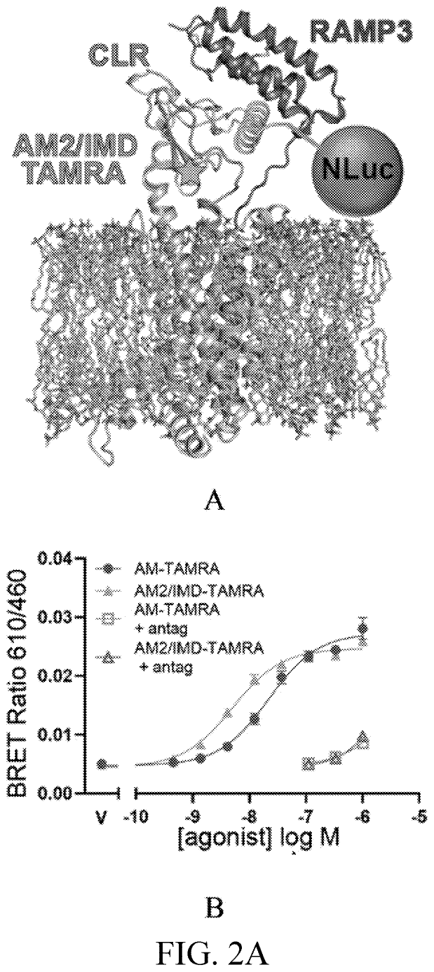

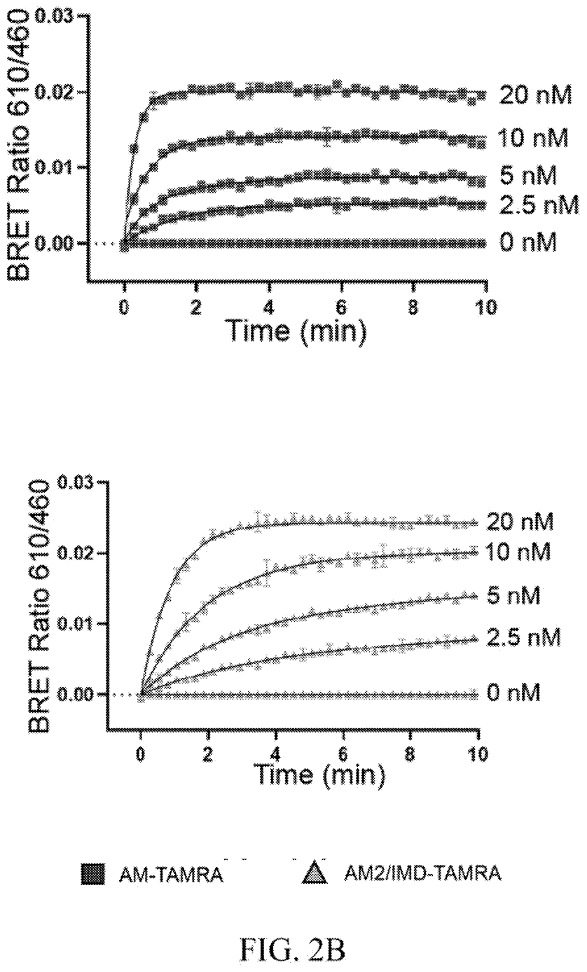

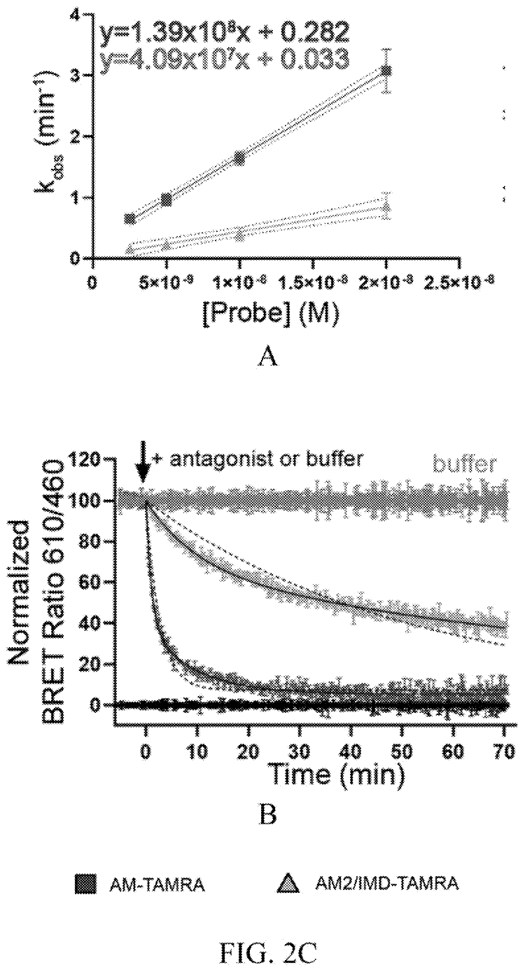

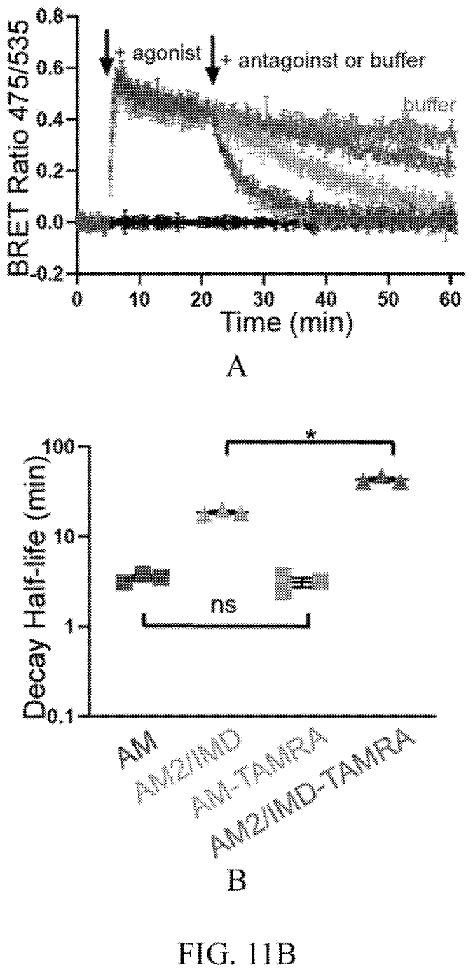

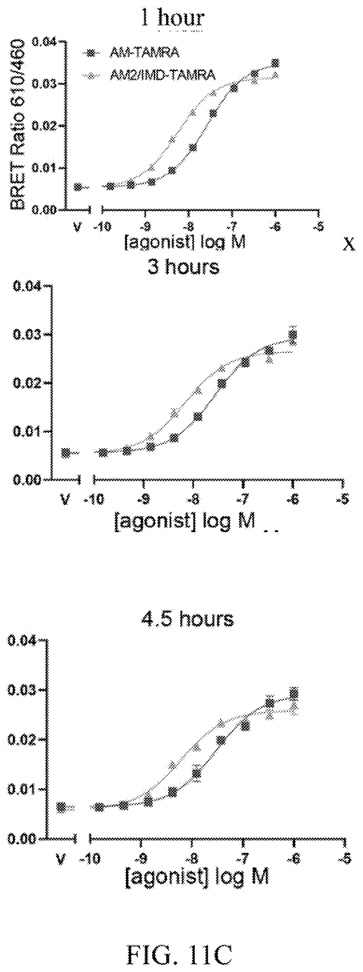

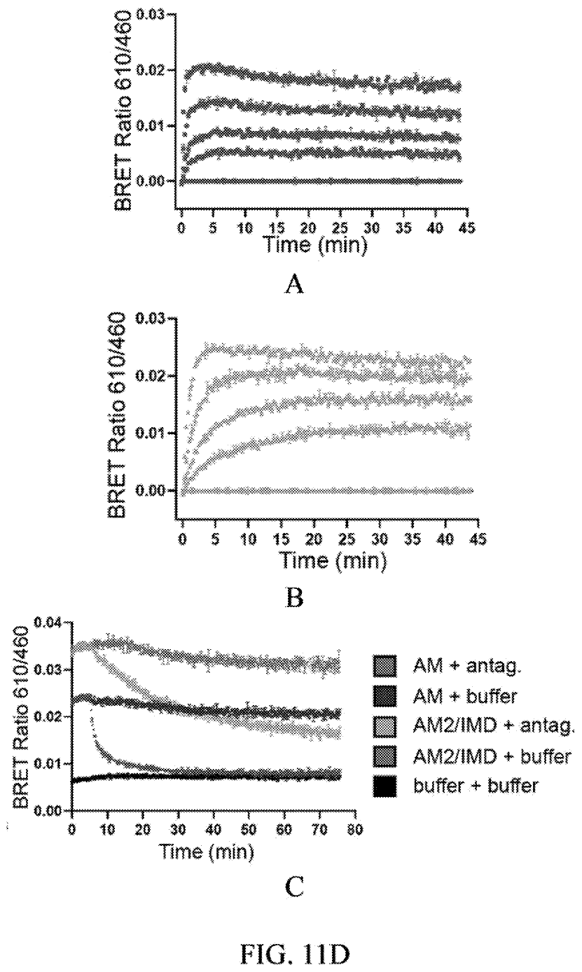

To test if AM2 receptor binding kinetics were responsible for its long-acting signaling phenotype, we used NanoBRET™ technology to compare the binding of AM and AM2 to the AM₂R. CLR was tagged with the BRET donor nanoluciferase (Nluc) at its N-terminus and we designed and ordered custom synthetic AM and AM2 peptides labeled with the acceptor fluorophore TAMRA on a lysine residue substituted at equivalent positions in AM (N40) and AM2 (D35) (FIG. 2A(A)). These residues were chosen based on their solvent-exposed locations in the crystal and cryo-EM structures and prior mutagenesis studies, which indicated that their substitution did not alter receptor ECD binding affinity. In concentration-response cAMP accumulation assays, wild-type pharmacology was observed for the Nluc-CLR-RAMP3 receptor with wild-type peptides (FIG. 11A(A)) and for the TAMRA-labeled peptides at untagged CLR-RAMP3 (FIG. 11A(B)). We further tested the labeled peptides in the real-time CAMYEL cAMP assay in COS-7 cells, which revealed wild-type behavior for AM-TAMRA (t_{1/2}~3 min) and a gain-of-function (slower decay) for AM2-TAMRA (t_{1/2}~43 min) (FIG. 11B(A,B)). These experiments indicated that the tagged receptor and peptides exhibited wild-type or near wild-type pharmacology and were thus suitable for NanoBRET™ binding studies.

Nluc-CLR was co-expressed with RAMP3 in COS-7 cells and membranes were prepared for the binding studies, which were conducted at 25° C. and in the presence of GTPγS to uncouple the receptor from G protein. Equilibrium binding experiments revealed saturable binding of AM-TAMRA and AM2-TAMRA that was blocked by excess unlabeled antagonist peptide, and the two agonists had binding affinities of 26 and 7 nM, respectively (FIG. 2A(B)), Table 8). Varying the incubation time indicated that 3 hr was sufficient to reach equilibrium (FIG. 11C(A)). In real-time association kinetics experiments AM-TAMRA reached equilibrium levels quicker than AM2-TAMRA (FIG. 2B(upper,lower)). Extended incubation revealed signal decay that we were unable to eliminate or correct for (FIG. 11D(A,B)), so we limited analysis of the association data to the first 10 min to minimize decay effects. The individual curves were best fit by a one-phase association exponential model and plots of the observed rates vs. probe concentration showed a linear relationship consistent with a single-step binding model ((FIG. 2C(A)). The on- and off-rates were determined as the slope and y-intercept, respectively. This revealed slower on- and off-rates and a longer half-life for AM2-TAMRA (t_{1/2}~41 min) as compared to AM-TAMRA (t_{1/2}~2.6 min) (Table 8). The K_{D} values calculated from the on- and off-rates were ~10-fold lower than the equilibrium K_{D} values. These discrepancies may reflect inaccuracies in fitting the kinetic data due to the signal decay issue and/or indicate that a single-step binding model does not appropriately describe these interactions. Nonetheless, the association data clearly indicated that AM2-TAMRA had slower binding kinetics than AM-TAMRA.

Next, we turned to dissociation kinetics experiments. The membranes were incubated with 10 nM of each probe for 25 min to reach equilibrium followed by injection of either buffer or 1 μM high-affinity unlabeled AM variant antagonist peptide to initiate dissociation. Signal decay was also evident in these experiments (FIG. 11D(C)), but the decay could be corrected for by normalization to the buffer control injection. These data revealed much slower dissociation of AM2-TAMRA than AM-TAMRA and the dissociation curves were best fit by a two-phase exponential decay model (FIG. 2C(B)). AM-TAMRA exhibited fast and slow component half-lives of 0.75 and 6.9 min, respectively, whereas AM2-TAMRA had fast and slow component half-lives of 7 and 76 min, respectively (Table 8). The fast components for AM-TAMRA and AM2-TAMRA accounted for 67% and 35% of the dissociation curves, respectively. These data are consistent with a two-step (un)binding process for both ligands as proposed for other class B GPCR peptide ligands, and indicate that AM2-TAMRA has a substantially longer AM₂R residence time than AM-TAMRA.

AM2 Promotes More Stable CLR-RAMP3 Complexes than AM

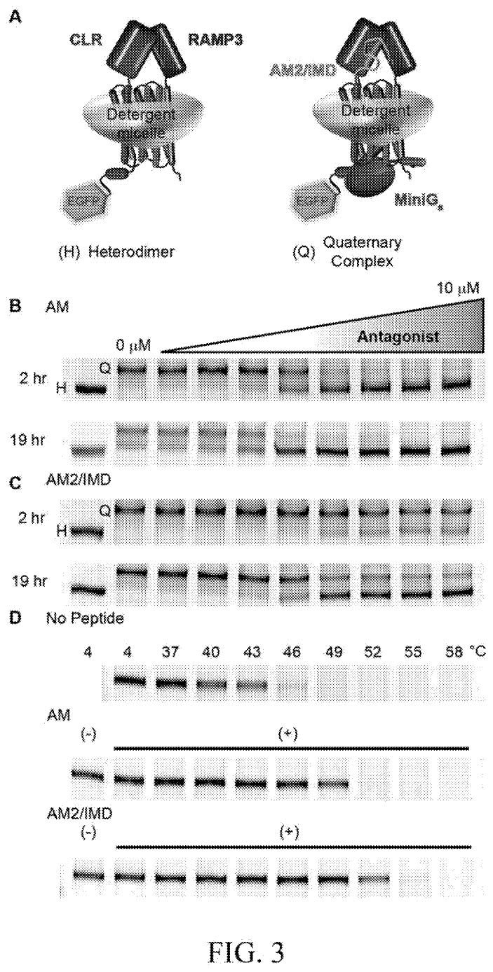

We previously reported a novel membrane protein native PAGE mobility shift assay for the biochemical characterization of agonist-dependent coupling of CLR-RAMP1-3 complexes to the G protein surrogate mini-Gs. Membranes co-expressing EGFP-tagged CLR and a RAMP are incubated with agonist and purified mini-Gs, solubilized with the gentle LMNG/CHS detergent system, and analyzed by native PAGE with in-gel fluorescence imaging. The heterodimeric CLR-RAMP and quaternary agonist-CLR-RAMP-miniGs complexes exhibit different mobilities due to their different sizes (FIG. 3(A)). AM and AM2 promoted formation of CLR-RAMP3-mini-Gs complexes with equal potencies in this assay. Here, we compared the stabilities of the AM- and AM2-promoted CLR-RAMP3-mini-Gs complexes to disruption by antagonist at 4° C. The membranes were incubated with AM or AM2 (200 nM) and excess mini-Gs for 30 min followed by a two-hour solubilization to form the quaternary complexes. The complexes were then challenged with increasing concentrations of a high-affinity AM variant peptide antagonist for 2 or 19 hrs followed by native PAGE analysis. The antagonist dose-dependently disrupted the AM quaternary complex with similar results observed with 2 or 19 hr antagonist exposure (FIG. 3(B)). Some breakdown of the AM quaternary complex was evident even in the absence of antagonist with the 19 hr incubation, indicating complex instability with extended incubation time. In contrast, the AM2 quaternary complex was much more resistant to breakdown in the presence of the antagonist (FIG. 3(C)). Remarkably, even after 19 hr exposure to 10 μM antagonist there was some AM2 quaternary complex remaining. These results indicated that AM2 promotes an AM₂R quaternary complex that is more resistant to disruption by antagonist than AM, which is consistent with AM2 having a slower off-rate from the G protein-coupled state of the receptor.

TABLE 8
Summary of NanoBRET ™ binding kinetic values.

| | | AM-TAMRA | AM2/IMD-TAMRA |
|---|---|---|---|
| Equilibrium | K_{D} (nM) | 26 ± 1.6 | 6.8 ± 0.81 |
| Binding | kₒₙ (M⁻¹min⁻¹ ± SEM)ⁱ | 1.4 × 10⁸ ± 1.1 × 10⁷ | 4.1 × 10⁷ ± 5.4 × 10⁶ |
| | k_{off} (min⁻¹ ± SEM)ⁱⁱ | 0.28 ± 0.045 | 0.032 ± 0.018 |
| Association | Calculated K_{D} | 2.1 ± 0.44 | 0.76 ± 0.33 |
| Kinetics | (nM ± SEM)ⁱⁱⁱ | | |
| | Time Constant τ | 3.8 ± 0.66 | 59 ± 30 |
| | (min ± SEM)^{iv} | | |
| | Half-life (min ± SEM)^{v} | 2.6 ± 0.46 | 41 ± 21 |
| | k_{off} fast (min⁻¹ ± SEM) | 0.93 ± 0.020 | 0.099 ± 0.0020 |
| | Time Constant τ fast | 1.1 ± 0.023 | 10 ± 0.20 |
| | (min ± SEM) | | |
| | Half-life fast (min ± SEM) | 0.75 ± 0.016 | 7.0 ± 0.14 |
| Dissociation | k_{off} slow (min⁻¹ ± SEM) | 0.10 ± 0.0031 | 0.0091 ± 0.00020 |
| Kinetics | Time Constant τ slow | 9.9 ± 0.31 | 110 ± 2.4 |
| | (min ± SEM) | | |
| | Half-life slow | 6.9 ± 0.21 | 76 ± 1.7 |
| | (min ± SEM) | | |
| | Percent Fast (% ± SEM) | 66.5 ± 1.2 | 34.7 ± 0.87 |

ⁱValues obtained from the slope in linear regression shown in FIG. 11D(B)
ⁱⁱValues obtained from the y-intercept in linear regression shown in FIG. 11D(B)
ⁱⁱⁱCalculated as off-rate/on-rate
^{iv}Calculated as the inverse of the off-rate
^{v}Calculated by In 2 divided by the off-rate

Next, we tested the thermostability of CLR-RAMP3 when bound to AM or AM2 in the absence of mini-Gs. The membranes were incubated in the absence or presence of 1 μM AM or AM2 for 30 min followed by detergent solubilization. The solubilized complexes were then incubated an additional 30 min at various temperatures followed by centrifugation and analysis of the supernatants by native PAGE. The agonists alone are too small to induce a mobility shift, but their binding was evidenced by the increased thermostability of the CLR-RAMP3 complex in their presence (FIG. 3(D)). The ligand-free AM₂R was stable up to ~43° C., whereas the AM- and AM2-bound AM₂R were stable to ~49° C. and ~52° C., respectively. The greater thermal stability in the presence of AM2 is consistent with it having a slower off-rate/longer residence time at the uncoupled AM₂R.

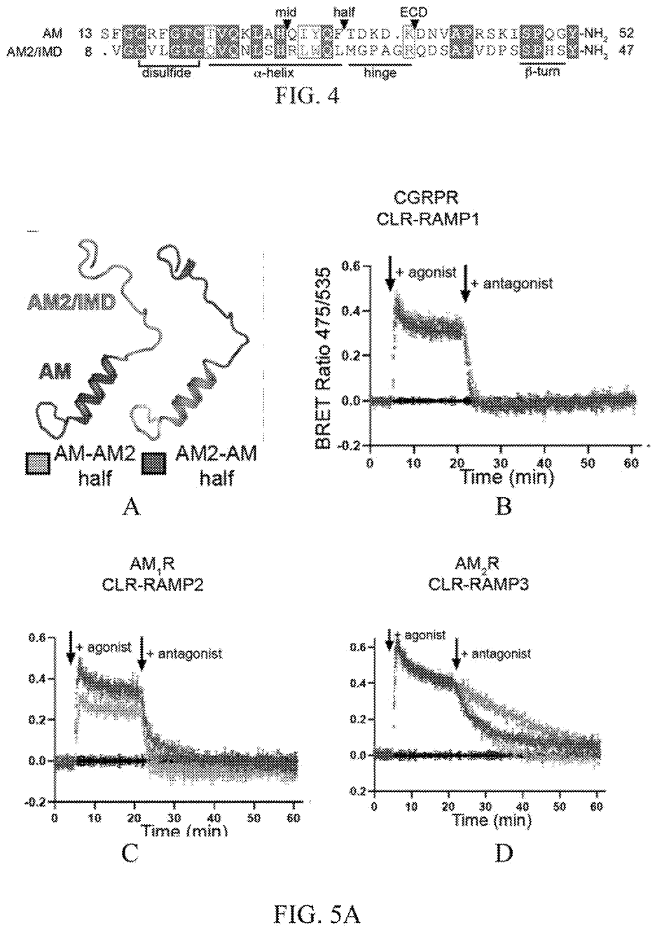

To determine the region of AM2 responsible for its slow off-rate and long-acting signaling behavior at AM₂R, we examined chimeras of AM and AM2 in the real-time cAMP signaling kinetics assay in COS-7 cells to assess their signal decay rates after antagonist challenge. The chimeras were compared to AM and AM2 controls within each experiment. First, we tested two previously described chimeras (A. M. Roehrkasse, et al., J. Biol. Chem. 295, 9736-9751 (2020)) with the junction at the half-way point at the end of the N-terminal α-helix (FIG. 4).

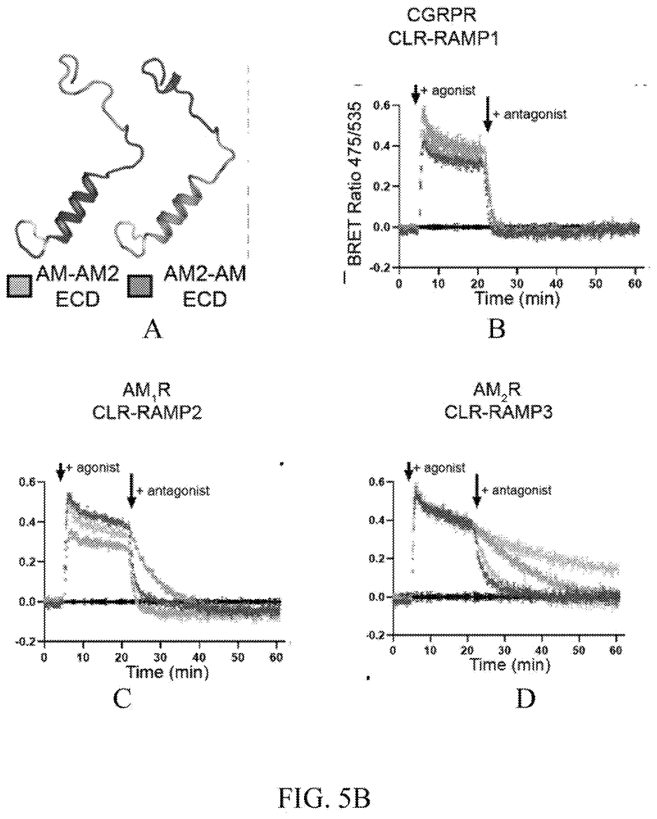

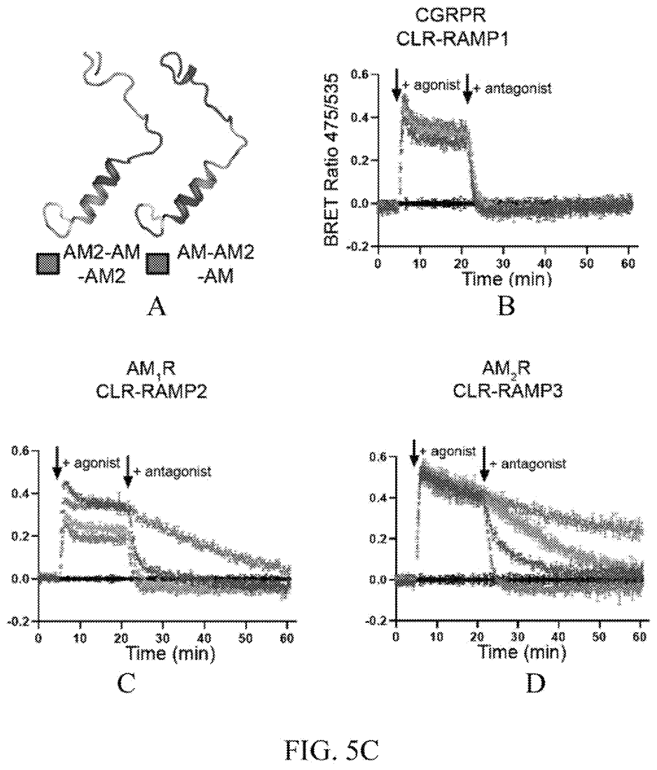

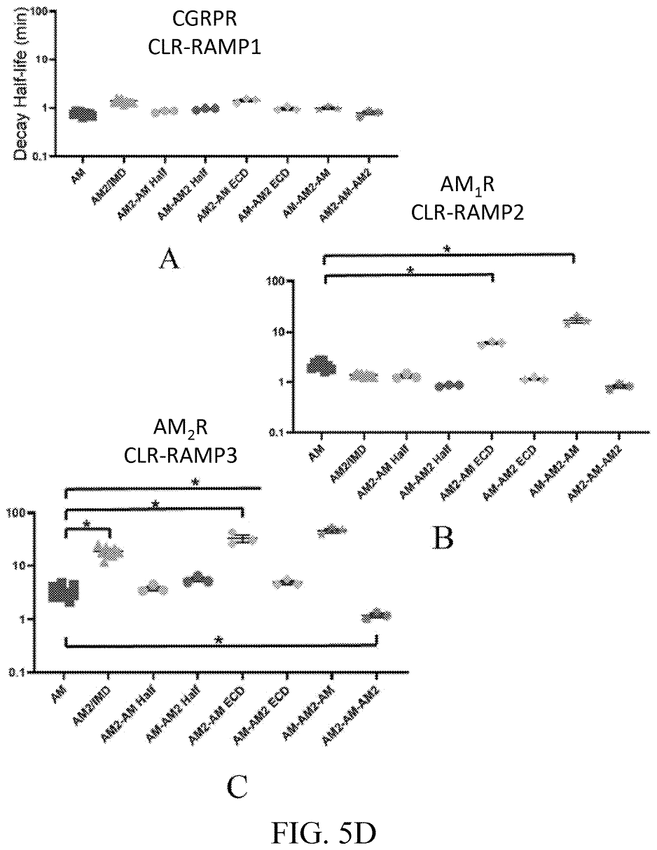

We refer to these as AM-AM2 half and AM2-AM half (FIG. 5A(A)). These exhibited decay half-lives similar to AM and AM2 at the CGRPR and AM₁R, and similar to AM at the AM₂R (FIGS. 5A(B-D), 5D(A-C); Table 9). Their lack of slow decay at the AM₂R indicated that the N- and C-terminal halves of AM2 were insufficient to confer the slow decay rate. Next, we tested new chimeras with a junction at the end of the central “hinge” region that connects the TMD and ECD binding portions (FIG. 4). We refer to these as AM-AM2 ECD and AM2-AM ECD because they have swapped ECD-binding regions (FIG. 5B(A)). AM-AM2 ECD exhibited decay half-lives similar to AM at each receptor, whereas AM2-AM ECD behaved like AM2 at the CGRPR and exhibited a gain-of-function slow decay at both AM₁R (t_{1/2}~6 min) and AM₂R (t_{1/2}~33 min) (FIGS. 5B(B-D), 5D(A-C); Table 9). These results indicated that the slow decay required the AM2 hinge region and additional residues prior to the hinge that were lacking in AM-AM2 half.

To define the central AM2 segment responsible for the slow decay, we tested peptide chimeras with swapped mid-regions. The N-terminal junction was after a conserved His in the α-helix (“mid” in FIG. 4) and the C-terminal junction was at the end of the hinge. The “mid” junction point was chosen because AM2 R23 forms a salt bridge with D96 at the “bottom” of the CLR ECD and we hypothesized that this interaction contributes to the slow decay. We refer to these chimeras as AM2-AM-AM2 and AM-AM2-AM (FIG. 5C(A)). AM2-AM-AM2 exhibited decay similar to AM at the CGRPR, and loss-of-function faster decay (as compared to AM) at both AM receptors (FIGS. 5C(B-D), 5D(A-C); Table 9). AM-AM2-AM had fast decay at the CGRPR and striking gain-of-function slower decay as compared to AM at AM₁R (t_{1/2}~17 min) and as compared to AM2 at AM₂R (t_{1/2}~46 min) (FIGS. 5C(B-D), 5D(A-C); Table 9). These results indicated that the AM2 central 11 residue segment from R23 to R33 is responsible for its slow off-rate at AM₂R. Remarkably, transfer of this segment into AM bestows upon it a slow off-rate, long duration signaling at both AM₁R and AM₂R.

TABLE 9
Summary of chimeric peptide cAMP signaling decay kinetic values

| | | Observed decay rate | Time Constant τ | Half-life |
|---|---|---|---|---|
| | | (min⁻¹ ± SEM) | (min ± SEM)* | (min ± SEM)** |
| CLR- | AM | 0.92 ± 0.033 | 1.1 ± 0.039 | 0.76 ± 0.027 |
| RAMP1 | AM2 | 0.50 ± 0.018 | 2.0 ± 0.075 | 1.4 ± 0.052 |
| | AM-AM2 half | 0.73 ± 0.021 | 1.4 ± 0.038 | 0.96 ± 0.027 |
| | AM2-AM half | 0.82 ± 0.027 | 1.2 ± 0.039 | 0.85 ± 0.027 |
| | AM-AM2 ECD | 0.72 ± 0.041 | 1.4 ± 0.086 | 0.98 ± 0.060 |
| | AM2-AM ECD | 0.48 ± 0.031 | 2.1 ± 0.13 | 1.4 ± 0.089 |
| | AM-AM2-AM | 0.70 ± 0.034 | 1.4 ± 0.074 | 1.0 ± 0.051 |
| | AM2-AM-AM2 | 0.89 ± 0.081 | 1.2 ± 0.099 | 0.80 ± 0.069 |
| CLR- | AM | 0.33 ± 0.020 | 3.1 ± 0.19 | 2.2 ± 0.13 |
| RAMP2 | AM2 | 0.51 ± 0.011 | 2.0 ± 0.042 | 1.4 ± 0.029 |
| | AM-AM2 half | 0.81 ± 0.021 | 1.2 ± 0.031 | 0.86 ± 0.021 |
| | AM2-AM half | 0.53 ± 0.035 | 1.9 ± 0.13 | 1.3 ± 0.092 |
| | AM-AM2 ECD | 0.60 ± 0.025 | 1.7 ± 0.074 | 1.2 ± 0.051 |
| | AM2-AM ECD | 0.12 ± 0.0056 | 8.7 ± 0.41 | 6.0 ± 0.29 |
| | AM-AM2-AM | 0.042 ± 0.0048 | 25 ± 3.0 | 17 ± 2.1 |
| | AM2-AM-AM2 | 0.86 ± 0.065 | 1.2 ± 0.089 | 0.82 ± 0.061 |
| CLR- | AM | 0.22 ± 0.019 | 4.8 ± 0.42 | 3.3 ± 0.29 |
| RAMP3 | AM2 | 0.039 ± 0.0030 | 27 ± 1.8 | 19 ± 1.3 |
| | AM-AM2 half | 0.13 ± 0.011 | 8.0 ± 0.72 | 5.6 ± 0.50 |
| | AM2-AM half | 0.19 ± 0.016 | 5.5 ± 0.50 | 3.8 ± 0.35 |
| | AM-AM2 ECD | 0.14 ± 0.011 | 7.0 ± 0.56 | 4.9 ± 0.39 |
| | AM2-AM ECD | 0.022 ± 0.0031 | 48 ± 7.3 | 33 ± 5.0 |
| | AM-AM2-AM | 0.015 ± 0.0015 | 66 ± 6.0 | 46 ± 4.2 |
| | AM2-AM-AM2 | 0.60 ± 0.049 | 1.7 ± 0.14 | 1.2 ± 0.099 |

*Time constant calculated as inverse of decay rate
**Half-life calculated as In 2 divided by decay rate

### The RAMP3 ECD Enables the Long-Acting Behavior of AM2

To identify the RAMP3 region that enables the AM2 slow off-rate and long-acting signaling, we constructed six domain swap chimeras of RAMP2 and RAMP3 and examined their properties in the real-time cAMP signaling kinetics assay in COS-7 cells. We swapped the ECD, the transmembrane helix domain (TMD), or the C-terminal cytoplasmic tail (“C-tail”) using the previously determined junction points. The chimeras were co-expressed with CLR and the AM and AM2 signaling kinetics were assessed as compared to AM₁R (RAMP2) and AM₂R (RAMP3) controls within each experiment. Strikingly, swapping the RAMP2/3 ECD completely swapped the AM2 slow decay phenotype, while having little to no effect on AM signaling kinetics (Table 8). In contrast, swapping the RAMP2/3 TMD (Table 8) or C-terminal tail (Table 8) had little to no effect on the signal decay observed for both agonists. The chimera results indicated that the slow decay of AM2 signaling at the AM₂R is dictated by the RAMP3 ECD.

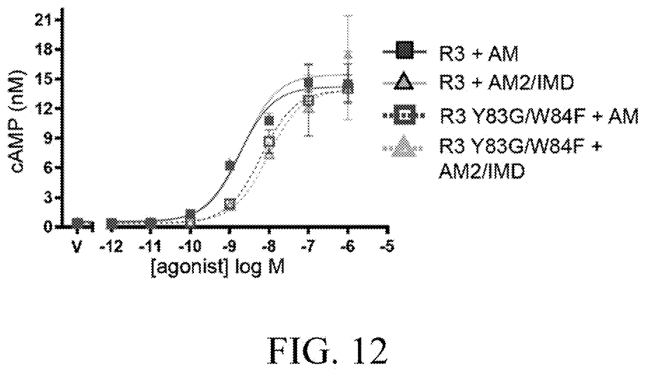

Next, we considered distinctions among the RAMP2/3 ECD residues that augment the CLR ECD binding pocket occupied by the peptide C-terminus. We previously showed that AM bound the purified RAMP2-CLR and RAMP3-CLR ECD complexes with nearly equal equilibrium affinities (K_{I~}5 μM), whereas AM2 bound the RAMP3-CLR ECD with an affinity (K_{I}~2 μM) 7-fold stronger than the RAMP2-CLR ECD (K_{I}~14 μM). These differences likely reflect, at least in part, the distinct RAMP2/3 residues that augment the binding site including RAMP2 G110, F111 and RAMP3 Y83, W84, which confer different contours to the binding pocket occupied by the peptide C-terminus. Notably, RAMP2/3 share E101/E74, which are important for binding AM. In the RAMP2 structure E101 directly contacts AM K46 and Y52. We reasoned that a RAMP3 Y83G/W84F double swap mutant would negatively affect AM2 more than AM, so we constructed and characterized this mutant. AM and AM2 were equipotent at the AM₂R with RAMP3 Y83G/W84F in a concentration-response cAMP accumulation assay, albeit with slight potency reductions as compared to wild type receptor (FIG. 12). In the CAMYEL cAMP signaling kinetics assay, AM kinetics were nearly wild type at the mutant receptor, whereas AM2 exhibited dramatically faster signal decay (t_{1/2}~3 min) that was nearly equal to that of AM (Table 10). In contrast, the AM-AM2-AM chimera, which contains the AM ECD binding region, retained its slow signal decay phenotype at the RAMP3 mutant AM₂R (t_{1/2}~37 min). These results strongly indicated that the RAMP3 ECD enabled the AM2 slow off-rate and long-acting signaling primarily via its role in forming the binding site for the peptide C-terminus.

TABLE 10
Summary of cAMP signaling decay kinetic values for RAMP2/3 chimeras and RAMP3 mutants.

| | AM | | | AM2 | | |
|---|---|---|---|---|---|---|
| | Observed | Time | | Observed | Time | |
| | decay rate | Constant τ | Half-life | decay rate | Constant τ | Half-life |
| | (min⁻¹ ± SEM) | (min ± SEM)^{‡} | (min ± SEM)^{§} | (min⁻¹ ± SEM) | (min ± SEM) | (min ± SEM) |
| RAMP Chimeras | | | | | | |
| RAMP2 | 0.34 ± 0.020 | 3.1 ± 0.13 | 2.1 ± 0.11 | 0.49 ± 0.036 | 2.1 ± 0.079 | 1.4 ± 0.097 |
| RAMP3 | 0.18 ± 0.011 | 5.5 ± 0.17 | 3.9 ± 0.25 | 0.035 ± 0.0047 | 29 ± 3.0 | 21 ± 3.3 |
| R2wR3 ECD | 0.22 ± 0.28 | 4.7 ± 0.53 | 3.2 ± 0.37 | 0.031 ± 0.0017 | 33 ± 1.7 | 23 ± 1.2 |
| R3wR2 ECD | 0.23 ± 0.016 | 4.3 ± 0.28 | 3.0 ± 0.19 | 0.36 ± 0.0038 | 2.8 ± 0.030 | 2.0 ± 0.021 |
| R2wR3 TMD | 0.27 ± 0.011 | 3.7 ± 0.16 | 2.6 ± 0.11 | 0.41 ± 0.015 | 2.4 ± 0.083 | 1.7 ± 0.058 |
| R3wR2 TMD | 0.16 ± 0.0066 | 6.3 ± 0.27 | 4.3 ± 0.19 | 0.037 ± 0.0084 | 30 ± 6.9 | 21 ± 4.8 |
| R2wR3 C-tail | 0.28 ± 0.026 | 3.7 ± 0.32 | 2.6 ± 0.22 | 0.54 ± 0.013 | 1.9 ± 0.044 | 1.3 ± 0.030 |
| R3wR2 C-tail | 0.22 ± 0.014 | 4.6 ± 0.30 | 3.2 ± 0.21 | 0.034 ± 0.0020 | 30 ± 1.8 | 21 ± 1.3 |
| RAMP3 Mutants | | | | | | |
| RAMP3 | 0.25 ± 0.013 | 4.0 ± 0.23 | 2.8 ± 0.16 | 0.048 ± 0.0038 | 22 ± 1.9 | 15 ± 1.3 |
| RAMP3 N58D | 0.22 ± 0.017 | 4.6 ± 0.35 | 3.2 ± 0.24 | 0.056 ± 0.0074 | 18 ± 2.2 | 13 ± 1.5 |
| RAMP3 N71S | 0.31 ± 0.015 | 3.3 ± 0.16 | 2.3 ± 0.11 | 0.075 ± 0.013 | 14 ± 2.2 | 9.7 ± 1.5 |
| RAMP3 | 0.28 ± 0.016 | 3.6 ± 0.19 | 2.5 ± 0.14 | 0.097 ± 0.0076 | 11 ± 0.77 | 7.3 ± 0.53 |
| N29D/N58D/N71S | | | | | | |
| RAMP3 | 0.40 ± 0.012 | 2.5 ± 0.070 | 1.7 ± 0.049 | 0.23 ± 0.0081 | 4.3 ± 0.16 | 3.0 ± 0.11 |
| Y83G/W84F | | | | | | |

^{‡}Time constant calculated as inverse decay rate
^{§}Half-life calculated as ln 2 divided by decay rate

### MD Simulations Reveal the Structural Basis for the AM2 Slow Off-Rate

To reveal the structural basis for the distinct AM2 binding kinetics at the AM₂R we sought to perform MD simulations to compare the AM- and AM2-bound AM₂R structures. This required resolving the issue of the different ECD-binding conformations of AM and AM2 in the cryo-EM and crystal structures. As described in Materials and Methods we rebuilt and re-refined the AM (6UUS) and AM2 (6UVA) AM₂R cryo-EM structures by using AlphaFold2 to obtain a RAMP3 ECD model combined with manual rebuilding as guided by fitting to the deposited cryo-EM density maps. This yielded structures with improved fits to the maps and showed that the AM and AM2 ECD-binding conformations in the cryo-EM structures were the same as those observed in the prior crystal structures. These improved structures served as the starting points for the MD simulations.

A set of three parallel molecular simulations were run for each of the AM-AM₂R and AM2-AM₂R systems. Each system included the full CLR and RAMP3 proteins and the bound peptide. The complex was embedded in an explicit membrane (40% cholesterol, 60% POPC) and included restraints on the intracellular CLR residues to mimic the presence of the G protein. To achieve better sampling of molecular conformations, we used a variant of the weighted ensemble algorithm called REVO (“Resampling Ensembles by Variation Optimization”) that periodically merges and clones trajectories in order to achieve a more diverse set of molecular structures. The criteria we chose to measure diversity—also referred to as the “distance metric”—was the RMSD of the peptide after alignment to the entire receptor dimer, which has been previously used to generate ligand unbinding trajectories. In the simulations here, both the TM and EC regions of the peptide remained associated with the receptor throughout, although the linker regions were flexible, allowing the ECD region of the receptor to move significantly with respect to the TM region in both cases.

We found that despite the significant motion of the ECD with respect to the TM domain, the hinge region of the peptide maintained stable hydrogen bonding interactions with residues in the CLR ECD loop region (residues 90 to 97) and the CLR N-terminal helix region (residues 36 to 38). The most stable interactions for each peptide were analyzed and it was found that AM2 maintains these interactions with higher probability. In particular, R33 played a central role in coordinating interactions with D90 of the CLR ECD loop region, as well as intramolecular hydrogen bonds with the backbone atoms of P30 and M28. As noted above, R23 also formed a strong salt-bridge interaction with D96 at the bottom of the CLR ECD; no analogous residue is present in AM. These interactions in AM2-AM₂R work together to stabilize the hinge region. After alignment to the hinge region, AM2-AM₂R showed lower RMSDs of both the CLR N-terminal helix and the CLR ECD loop region than AM-AM₂R. This reveals a more stable bound complex which could contribute to the longer observed residence time of AM2.

The flexibility and molecular interactions in the ECD complexes were analyzed. By aligning the set of 144 final structures from the REVO-MD simulations using the CLR ECD, we observed a slight increase in stability of the C-terminal end of AM2 in comparison to AM. The entire ensemble of AM and AM2 conformations was analyzed quantitatively using different RMSD measurements and alignments. For each domain, with each possible alignment, we found larger conformational changes in the AM-bound system in comparison to the AM2-bound system. The stabilizing interactions observed in the molecular dynamics simulations were further investigated, showing representative high-weight frames from the end of the REVO-MD simulation. We also computed the density of water molecules after alignment to a set of residues surrounding the terminal tyrosine in the peptide and W84 of RAMP3. The AM2-bound system was stabilized by a set of water-mediated hydrogen bonds involving G71 (CLR), Y47 (AM2), E74 (RAMP3) and the backbone nitrogen of W84 (RAMP3). This involved three labeled water molecules, which all reside in the highest-density solvation pockets. K46 of AM, while introducing a powerful salt-bridge interaction, disrupts this water-mediated H-bond network. Significantly, we also observed a stable hydrogen bond between the side chains of W84 and T73. While this is present in both systems, we found this is more stable in the AM2 system, likely due to the stabilizing interactions afforded by the water-mediated hydrogen bond network. This is consistent with the effects of the RAMP3 W84F mutation described above, which would eliminate this H-bond and destabilize the water-mediated H-bond network that is specific to the AM2 system.

Given the results from our chimeric peptide agonist signaling kinetics data and our elucidation of the mechanism of the AM2 slow off-rate at the AM₂R, we sought to engineer high-affinity peptide antagonists with kinetic selectivity for the AM₂R. A kinetically selective antagonist would have a slow off-rate/long residence time only at the AM₂R such that with extended time it would selectively antagonize only the AM₂R and not the CGRPR or AM₁R. Antagonist versions of the CGRP family peptides were created by truncating the N-terminal disulfide loop region, which is required for receptor activation. Novel antagonist designs began with the truncated peptide backbones AM2(17-47)NH₂ (SEQ ID NO:8), AM2(17-33)-AM(39-52)NH₂ (SEQ ID NO:22) chimera, or AM(22-28)-AM2(23-33)-AM(39-52)NH₂ (SEQ ID NO:27) chimera. Various amino acid substitutions were added which further increased affinities for the RAMP3-CLR ECD complex (AM₂R).

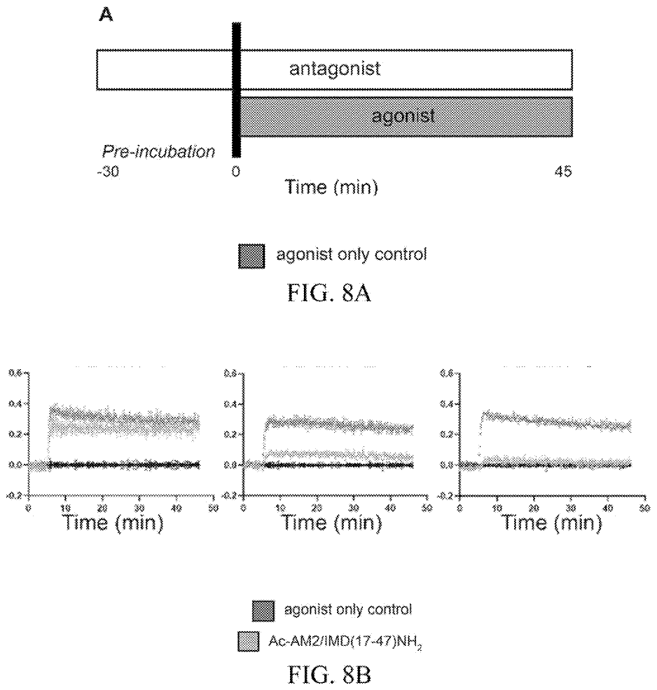

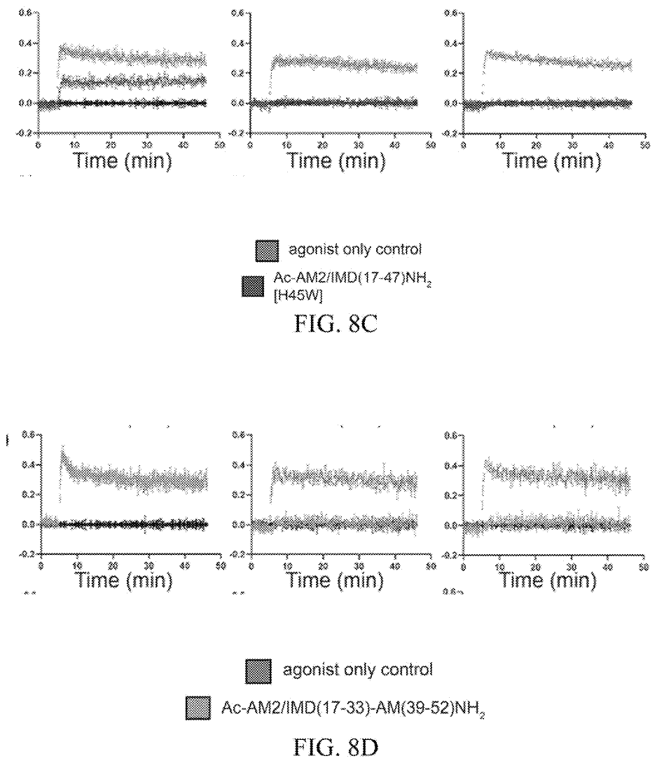

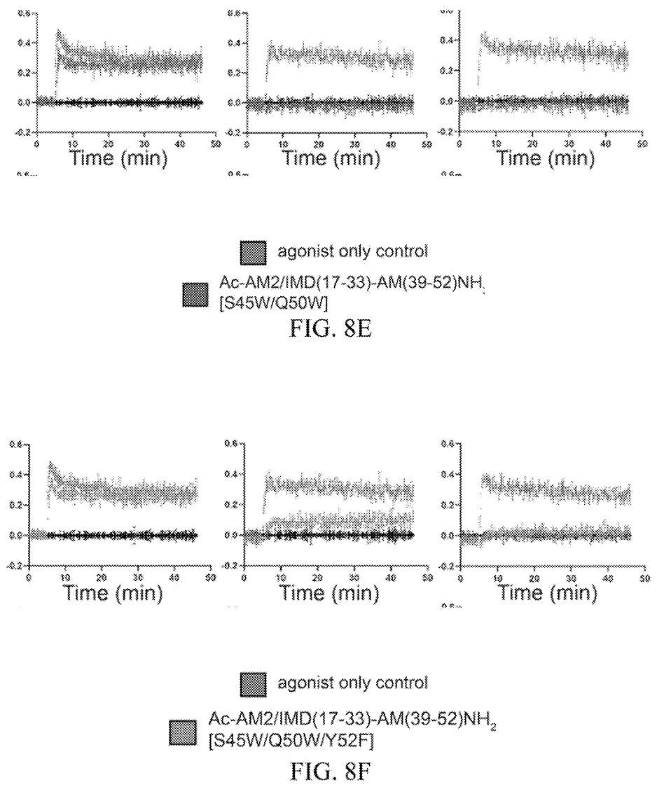

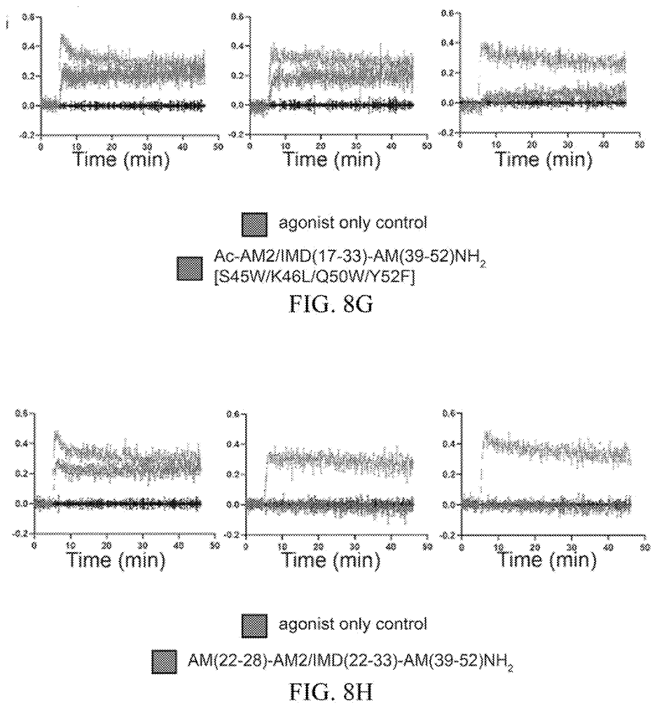

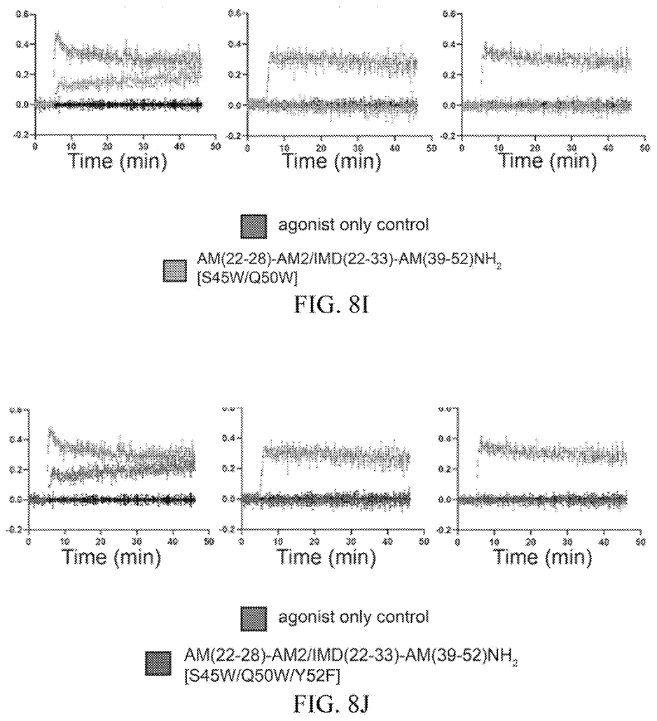

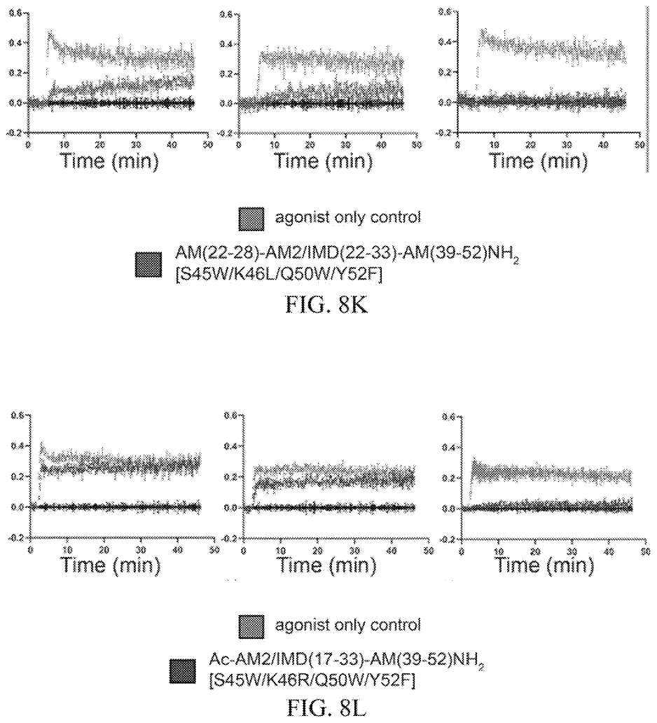

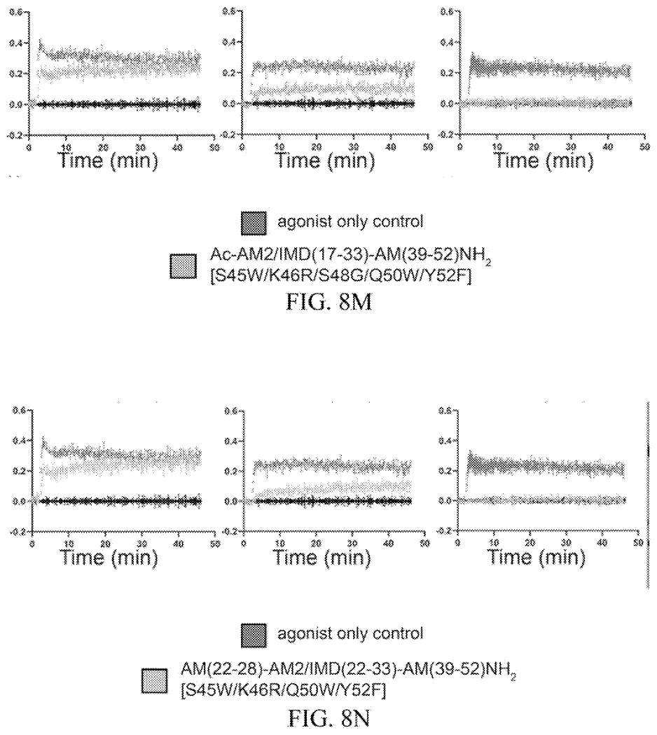

First, we tested the ability of each antagonist to antagonize cAMP signaling at each CLR-RAMP complex using the CAMYEL cAMP signaling kinetics assay in which cells expressing the receptors were pre-incubated with the antagonists followed by agonist co-addition (FIG. 8A). In this assay format, most of the antagonists had weak antagonist activity at CLR-RAMP1, while antagonizing both the CLR-RAMP2 and -RAMP3 complexes (FIGS. 8B-8N).

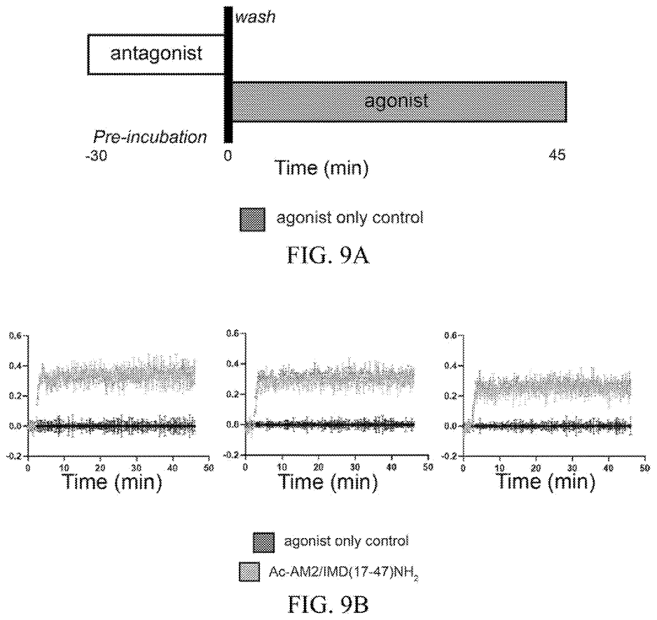

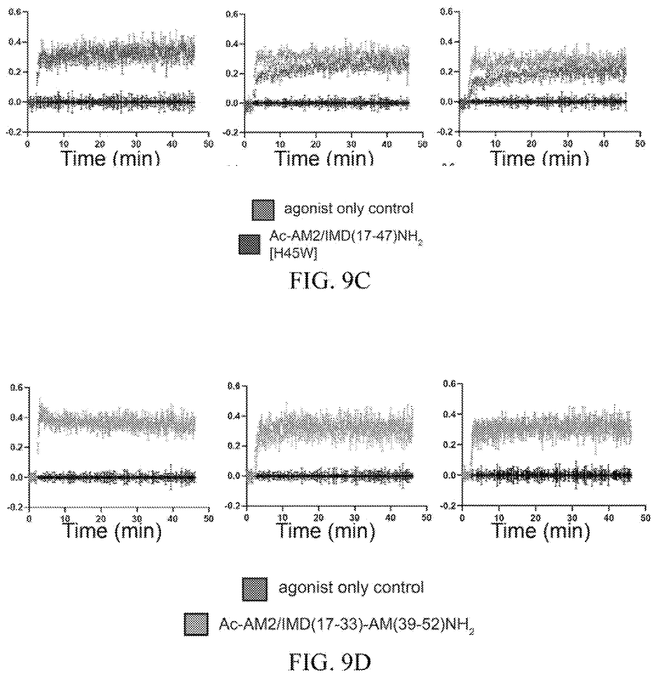

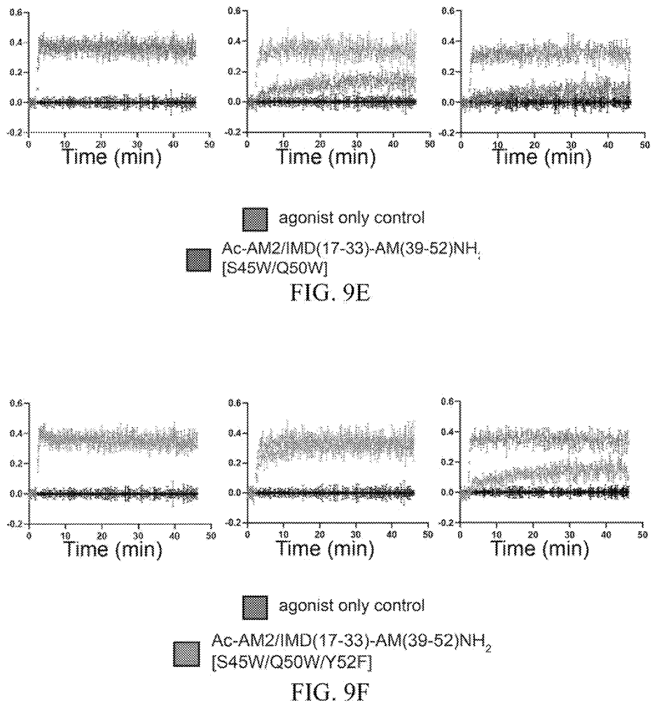

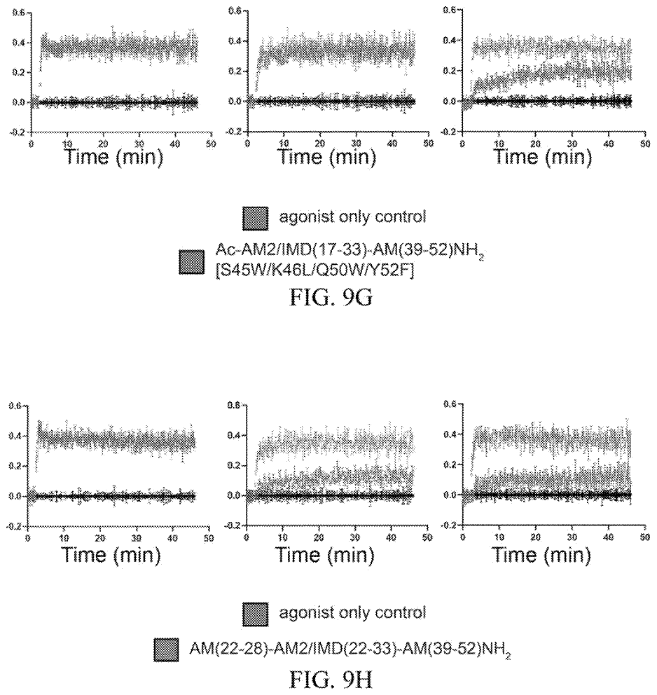

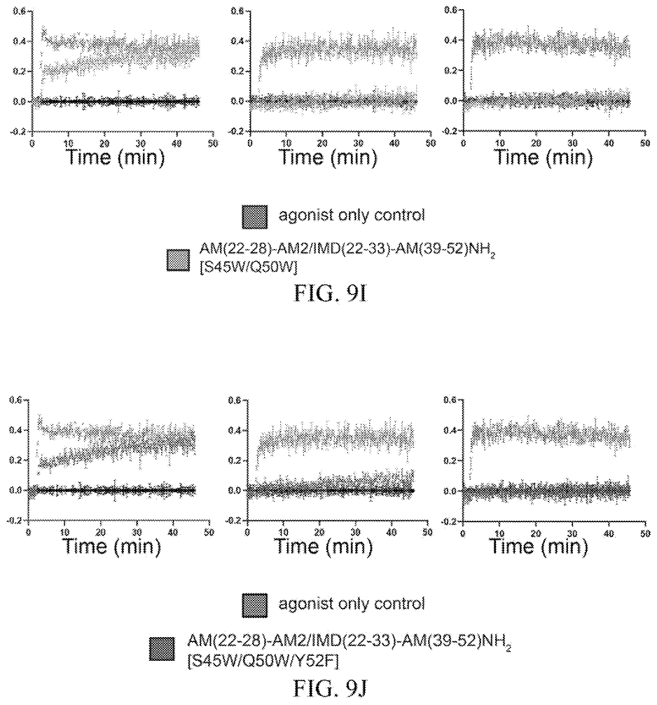

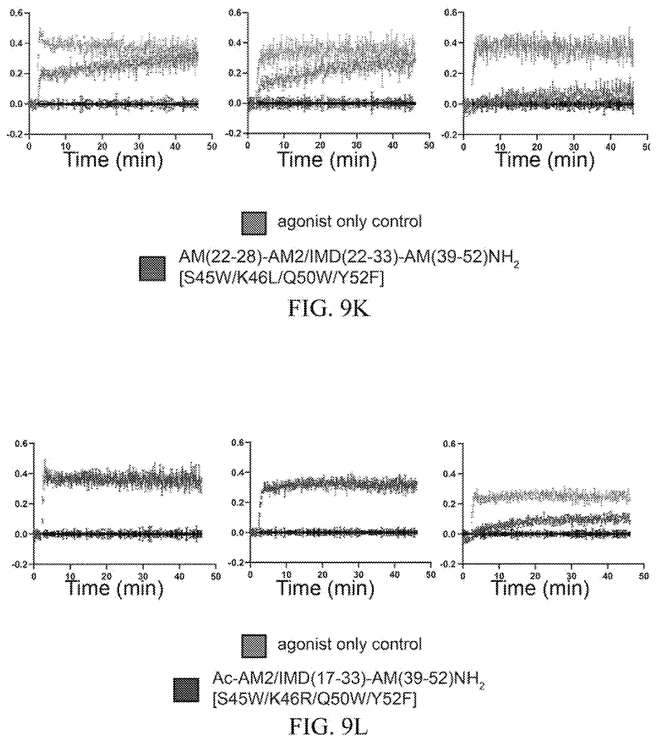

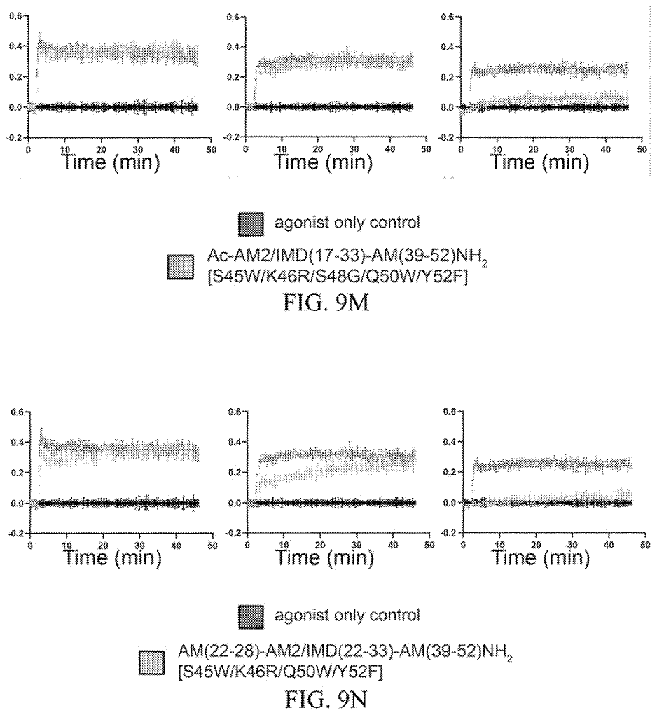

We then tested these antagonists in a wash-out assay format in which the cells were pre-incubated with antagonists, followed by antagonist washout and then agonist addition (FIG. 9A). Results are shown in FIGS. 9B-9N. In this assay format only antagonists with a slow off-rate/long residence time will continue to antagonize the receptor after washout. This revealed that the N-terminally acetylated peptides AM2(17-33)-AM(39-52)NH₂[S45W/Q50W/Y52F] (SEQ ID NO:24) (FIG. 9F), AM2(17-33)-AM(39-52)NH₂ [S45W/K46L/Q50W/Y52F] (SEQ ID NO:25) (FIG. 9G), and AM(22-28)-AM2(23-33)-AM(39-52)NH₂ [S45W/K46L/Q50W/Y52F](FIG. 9K) (SEQ ID NO:30), AM2(17-33)-AM(39-52)NH₂ [S45W/K46R/Q50W/Y52F](FIG. 9L) (SEQ ID NO:69), AM2(17-33)-AM(39-52)NH₂ [S45W/K46R/S48G/Q50W/Y52F](FIG. 9M) (SEQ ID NO:71), and AM(22-28)-AM2(23-33)-AM(39-52)NH₂ [S45W/K46R/Q50W/Y52F] (SEQ ID NO:84) (FIG. 9N) selectively antagonized only the CLR-RAMP3 complex (AM₂R) after washout. Thus, these peptides are non-limiting examples of novel high-affinity antagonists that are kinetically selective for the AM₂R.

### II.

### Methods

### Plasmids

The wild-type human CLR, RAMP1, RAMP2, and RAMP3 expression plasmids in the pcDNA3.1 vector backbone were obtained from the cDNA Resource Center. The wild-type mouse CLR, RAMP1, RAMP2, and RAMP3 expression plasmids were constructed by using the Gibson assembly method to insert a synthetic DNA fragment encoding the desired mouse protein into the pcDNA3.1 expression vector. The synthetic genes were ordered from Thermofisher geneart and were not codon optimized. The coding sequences of these plasmids were confirmed by automated DNA sequencing at the OUHSC sequencing core facility. The CAMYEL biosensor plasmid was previously described. Purified plasmids for cell transfections were prepared with the Machery-Nagel midiprep kit according to the manufacturer's directions.

### Cell Culture

COS-7 cells were obtained from ATCC and were cultured in DMEM+10% FBS in a humidified CO₂ incubator at 37° C. Cells were passaged twice per week.

COS-7 cells were seeded at 20,000 cells/well in a 96-well plate and transiently transfected the next day with the indicated CLR, RAMP, and CAMYEL plasmids using branched PEI transfection reagent. Two days after transfection the cells were stimulated with the indicated agonist peptides for 30 min at 37° C. followed by measurement of the CAMYEL BRET signals in a BMG Labtech polarstar omega or pherastar plate reader.

Real-Time cAMP Signaling Duration Assay Using CAMYEL cAMP Biosensor

COS-7 cells were seeded at 20,000 cells/well in a 96-well plate and transiently transfected the next day with the indicated CLR, RAMP, and CAMYEL plasmids using branched PEI transfection reagent. Two days after transfection the assay was conducted at room temperature. After establishing a baseline cAMP signal, agonist (100 nM unless otherwise noted) was manually added with continued reading of the BRET signal for ~15 minutes, followed by manual addition of buffer control or excess peptide antagonist (as indicated) to initiate the cAMP decay phase, which was monitored for ~80 min. The BRET signals were measured in a BMG Labtech polarstar omega or pherastar plate reader.

### Data Analysis

The cAMP BRET ratio was calculated as the 475 nm signal/535 nm signal so that an increase in BRET ratio corresponded to an increase in cAMP concentration. Background from buffer control samples was subtracted. The dose-response data were fitted by nonlinear regression to a 3-parameter dose-response stimulation equation in GraphPad Prism to determine the potency (pEC₅₀). The cAMP decay phase portion of the real-time cAMP signaling duration assay data were fitted by nonlinear regression to a one-phase exponential decay equation in GraphPad Prism to determine the decay rate, half-life, and time constant. The reported values represent mean+/−SEM from at least 3 independent biological replicates performed on different days.

### Results

cAMP Signaling Potency of the Engineered AM-AM2 Chimera Variants.

The cAMP signaling potencies of the engineered AM-AM2 chimera variants were determined for the human and mouse CLR-RAMP receptor complexes heterologously expressed in COS-7 cells (Table 10). The three engineered variants, ssAM, a.k.a. AM(13-52)NH₂[S48G/Q50W](SEQ ID NO:52), ssAM-AM2-AM, a.k.a., AM(13-27)-AM2(23-33)-AM(39-52)NH₂ [S48G/Q50W](SEQ ID NO:51), and ssAM2-AMECD, a.k.a., AM2(8-33)-AM-ECD(39-52)NH₂ [S48G/Q50W](SEQ ID NO:53), exhibited potencies that differed from their wild-type counterparts by ~10-fold change or less. ssAM-AM2-AM and ssAM2-AMECD were slightly less selective towards the two AM receptors (CLR-RAMP2/3) than wild-type AM. The peptides behaved similarly at the human and mouse receptors. Overall, these data indicated that the engineered variants retained reasonably strong cAMP signaling potencies at both human and mouse CLR-RAMP complexes, albeit with slightly reduced receptor selectivity, at least as assessed in these endpoint equilibrium assays at a single 30-minute timepoint.

cAMP Signal Duration of the Engineered AM-AM2 Chimera Variants

The cAMP signaling durations of the engineered AM-AM2 chimera variants were determined for the human and mouse CLR-RAMP receptor complexes heterologously expressed in COS-7 cells (Table 11). These were compared to the wild type AM peptide, which is short-acting at each CLR-RAMP complex, and to wild type AM2, which is a naturally long-acting agonist of the CLR-RAMP3 complex (AM₂R). For the human CLR-RAMP complexes, ssAM was long-acting at the CLR-RAMP2/3 (AM₁/2R) complexes (half-lives ~20-30 min) and short-acting at the CLR-RAMP1 (CGRPR) complex (half-life ~1 min). ssAM-AM2-AM and ssAM2-AMECD behaved similar to each other in these assays, with both being extremely long-acting at the two AM receptors (CLR-RAMP2/3). Indeed, these peptides were so long-acting that we could not determine their cAMP signal duration half-lives because little to no decay was observed over the time course of the decay phase of the assay (~80 minutes). Notably, ssAM-AM2-AM and ssAM2-AMECD exhibited kinetic selectivity for the two AM receptors over the CGRP receptor (CLR-RAMP1) at which they had cAMP decay half-lives of only ~8-10 minutes. Similar results were observed for the 3 engineered “ss” variants at the mouse receptors, with the exception that they were longer acting at the mouse CGRPR than the human CGRPR. Overall, these data indicated that the engineered variants are long-acting by exhibiting sustained cAMP signaling at the two AM receptors (CLR-RAMP2/3), while retaining kinetic selectivity for the AM receptors over the CGRPR.

TABLE 11
cAMP equilibrium and kinetic values at the human and mouse CLR-RAMP receptors.

| | | human | | | mouse | | |
|---|---|---|---|---|---|---|---|
| | | CGRPR | AM₁R** | AM₂R* | CGRPR | AM₁R* | AM₂R* |
| AM(13-52) | Rate (min⁻¹ ± | 0.94 ± | 0.42 ± | N.D.^{††} | 1.04 ± | 0.36 ± | N.D. |
| | SEM) | 0.14 | 0.02 | | 0.13 | 0.02 | |
| | Half-life | 0.77 ± | 1.66 ± | N.D. | 0.68 ± | 1.91 ± | N.D. |
| | (min ± SEM)^{‡‡} | 0.12 | 0.09 | | 0.07 | 0.11 | |
| | Time constant | 1.11 ± | 2.39 ± | N.D. | 0.99 ± | 2.76 ± | N.D. |
| | (min ± SEM)^{§§} | 0.18 | 0.12 | | 0.11 | 0.16 | |
| | pEC₅₀ (±SEM) | 7.98 ± | 9.25 ± | N.D. | 8.10 ± | 9.31 ± | N.D. |
| | | 0.08 | 0.04 | | 0.06 | 0.08 | |
| AM2(8-47)*** | Rate (min⁻¹ ± | N.D. | N.D. | 0.026 ± | 0.122 ± | 0.031 ± | 0.0048 ± |
| | SEM) | | | 0.003 | 0.008 | 0.004 | 0.0005 |
| | Half-life | N.D. | N.D. | 26.83 ± | 5.74 ± | 23.21 ± | 151.00 ± |
| | (min ± SEM) | | | 2.82 | 0.40 | 2.56 | 12.99 |
| | Time constant | N.D. | N.D. | 38.71 ± | 8.29 ± | 33.49 ± | 217.8 ± |
| | (min ± SEM) | | | 4.06 | 0.58 | 3.70 | 18.74 |
| | pEC₅₀ (±SEM) | N.D. | N.D. | 8.92 ± | N.D. | N.D. | 9.05 ± |
| | | | | 0.001 | | | 0.03 |
| AM(13-52) | Rate (min⁻¹ ± | 0.67 ± | 0.024 ± | 0.032 ± | 0.26 ± | 0.008 ± | 0.011 ± |
| S48G/Q50W | SEM) | 0.04 | 0.0001 | 0.004 | 0.01 | 0.001 | 0.001 |
| “ssAM” | Half-life | 1.04 ± | 28.57 ± | 22.67 ± | 2.68 ± | 91.44 ± | 66.82 ± |
| | (min ± SEM) | 0.07 | 0.15 | 2.60 | 0.14 | 13.47 | 5.15 |
| | Time constant | 1.50 ± | 41.22 ± | 32.71 ± | 3.87 ± | 131.9 ± | 96.43 ± |
| | (min ± SEM) | 0.10 | 0.22 | 3.76 | 0.21 | 19.44 | 7.43 |
| | pEC₅₀ (±SEM) | 8.40 ± | 9.03 ± | 8.77 ± | 8.80 ± | 8.86 ± | 8.95 ± |
| | | 0.03 | 0.06 | 0.03 | 0.04 | 0.05 | 0.05 |
| AM(13-28)- | Rate (min⁻¹ ± | 0.073 ± | N.M.^{†††} | N.M. | 0.016 ± | N.M.^{‡‡‡} | N.M. |
| AM2(23-33)- | SEM) | 0.001 | | | 0.003 | | |
| AM(39-52) | Half-life | 9.50 ± | N.M. | N.M. | 45.10 ± | N.M. | N.M. |
| S48G/Q50W | (min ± SEM) | 0.08 | | | 7.07 | | |
| “ssAM- | Time constant | 13.71 ± | N.M. | N.M. | 65.08 ± | N.M. | N.M. |
| AM2-AM” | (min ± SEM) | 0.11 | | | 120.20 | | |
| | pEC₅₀ (±SEM) | 8.82 ± | 8.41± | 8.18 ± | 9.07 ± | 8.37 ± | 8.22 ± |
| | | 0.07 | 0.08 | 0.04 | 0.06 | 0.07 | 0.03 |
| AM2(8-33)- | Rate (min⁻¹ ± | 0.087 ± | N.M. | N.M. | 0.018 ± | N.M. | N.M. |
| AM(39-52) | SEM) | 0.0004 | | | 0.002 | | |
| S48G/Q50W | Half-life | 7.97 ± | N.M. | N.M. | 38.77 ± | N.M. | N.M. |
| “ssAM2- | (min ± SEM) | 0.04 | | | 4.76 | | |
| AMECD” | Time constant | 11.50 ± | N.M. | N.M. | 55.94 ± | N.M. | N.M. |
| | (min ± SEM) | 0.06 | | | 6.86 | | |
| | pEC₅₀ (±SEM) | 8.83 ± | 8.59 ± | 8.64 ± | 9.27 ± | 8.58 ± | 8.75 ± |
| | | 0.09 | 0.04 | 0.02 | 0.09 | 0.02 | 0.09 |

**Kinetic rate curve fits were constrained to plateau at zero except for AM(13-52)
^{††}N.D. = not done
^{‡‡}Half-life calculated by ln 2/rate
^{§§}Time constant calculated by 1/rate
***AM2(8-47) cAMP kinetics at mouse CLR-RAMP3 n = 6. All other values in the table are n = 3.
^{†††}N.M. = not measurable because the peptides exhibit no decay in cAMP signal over the time of assay
^{‡‡‡}N.M. = not measurable because the peptides exhibit no decay in cAMP signal over the time of assay

TABLE 12
Examples of variants of SEQ ID NO: 21 (where
X₁ is V, X₂ is Q, X₃ is N, X₅ is S, X₆ is H)

| | | | | | Peptide |
|---|---|---|---|---|---|
| X₂₄ | X₂₅ | X₂₇ | X₂₉ | X₃₁ | Designation |
| S | K | S | Q | Y | SKSQY_{V} |
| S | K | S | Q | F | SKSQF_{V} |
| S | K | S | W | Y | SKSWY_{V} |
| S | K | S | W | F | SKSWF_{V} |
| S | K | G | Q | Y | SKGQY_{V} |
| S | K | G | Q | F | SKGQF_{V} |
| S | K | G | W | Y | SKGWY_{V} |
| S | K | G | W | F | SKGWF_{V} |
| S | L | S | Q | Y | SLSQY_{V} |
| S | L | S | Q | F | SLSQF_{V} |
| S | L | S | W | Y | SLSWY_{V} |
| S | L | S | W | F | SLSWF_{V} |
| S | L | G | Q | Y | SLGQY_{V} |
| S | L | G | Q | F | SLGQF_{V} |
| S | L | G | W | Y | SLGWY_{V} |
| S | L | G | W | F | SLGWF_{V} |
| S | R | S | Q | Y | SRSQY_{V} |
| S | R | S | Q | F | SRSQF_{V} |
| S | R | S | W | Y | SRSWY_{V} |
| S | R | S | W | F | SRSWF_{V} |
| S | R | G | Q | Y | SRGQY_{V} |
| S | R | G | Q | F | SRGQF_{V} |
| S | R | G | W | Y | SRGWY_{V} |
| S | R | G | W | F | SRGWF_{V} |
| W | K | S | Q | Y | WKSQY_{V} |
| W | K | S | Q | F | WKSQF_{V} |
| W | K | S | W | Y | WKSWY_{V} |
| W | K | S | W | F | WKSWF_{V} |
| W | K | G | Q | Y | WKGQY_{V} |
| W | K | G | Q | F | WKGQF_{V} |
| W | K | G | W | Y | WKGWY_{V} |
| W | K | G | W | F | WKGWF_{V} |
| W | L | S | Q | Y | WLSQY_{V} |
| W | L | S | Q | F | WLSQF_{V} |
| W | L | S | W | Y | WLSWY_{V} |
| W | L | S | W | F | WLSWF_{V} |
| W | L | G | Q | Y | WLGQY_{V} |
| W | L | G | Q | F | WLGQF_{V} |
| W | L | G | W | Y | WLGWY_{V} |
| W | L | G | W | F | WLGWF_{V} |
| W | R | S | Q | Y | WRSQY_{V} |
| W | R | S | Q | F | WRSQF_{V} |
| W | R | S | W | Y | WRSWY_{V} |
| W | R | S | W | F | WRSWF_{V} |
| W | R | G | Q | Y | WRGQY_{V} |
| W | R | G | Q | F | WRGQF_{V} |
| W | R | G | W | Y | WRGWY_{V} |
| W | R | G | W | F | WRGWF_{V} |

TABLE 13
Examples of variants of SEQ ID NO: 21 (where
X₁ is T, X₂ is Q, X₃ is N, X₅ is S, X₆ is H)

| | | | | | Peptide |
|---|---|---|---|---|---|
| X₂₄ | X₂₅ | X₂₇ | X₂₉ | X₃₁ | Designation |
| S | K | S | Q | Y | SKSQY_{T} |
| S | K | S | Q | F | SKSQF_{T} |
| S | K | S | W | Y | SKSWY_{T} |
| S | K | S | W | F | SKSWF_{T} |
| S | K | G | Q | Y | SKGQY_{T} |
| S | K | G | Q | F | SKGQF_{T} |
| S | K | G | W | Y | SKGWY_{T} |
| S | K | G | W | F | SKGWF_{T} |
| S | L | S | Q | Y | SLSQY_{T} |
| S | L | S | Q | F | SLSQF_{T} |
| S | L | S | W | Y | SLSWY_{T} |
| S | L | S | W | F | SLSWF_{T} |
| S | L | G | Q | Y | SLGQY_{T} |
| S | L | G | Q | F | SLGQF_{T} |
| S | L | G | W | Y | SLGWY_{T} |
| S | L | G | W | F | SLGWF_{T} |
| S | R | S | Q | Y | SRSQY_{T} |
| S | R | S | Q | F | SRSQF_{T} |
| S | R | S | W | Y | SRSWY_{T} |
| S | R | S | W | F | SRSWF_{T} |
| S | R | G | Q | Y | SRGQY_{T} |
| S | R | G | Q | F | SRGQF_{T} |
| S | R | G | W | Y | SRGWY_{T} |
| S | R | G | W | F | SRGWF_{T} |
| W | K | S | Q | Y | WKSQY_{T} |
| W | K | S | Q | F | WKSQF_{T} |
| W | K | S | W | Y | WKSWY_{T} |
| W | K | S | W | F | WKSWF_{T} |
| W | K | G | Q | Y | WKGQY_{T} |
| W | K | G | Q | F | WKGQF_{T} |
| W | K | G | W | Y | WKGWY_{T} |
| W | K | G | W | F | WKGWF_{T} |
| W | L | S | 0 | Y | WLSQY_{T} |
| W | L | S | Q | F | WLSQF_{T} |
| W | L | S | W | Y | WLSWY_{T} |
| W | L | S | W | F | WLSWF_{T} |
| W | L | G | Q | Y | WLGQY_{T} |
| W | L | G | Q | F | WLGQF_{T} |
| W | L | G | W | Y | WLGWY_{T} |
| W | L | G | W | F | WLGWF_{T} |
| W | R | S | Q | Y | WRSQY_{T} |
| W | R | S | Q | F | WRSQF_{T} |
| W | R | S | W | Y | WRSWY_{T} |
| W | R | S | W | F | WRSWF_{T} |
| W | R | G | Q | Y | WRGQY_{T} |
| W | R | G | Q | F | WRGQF_{T} |
| W | R | G | W | Y | WRGWY_{T} |
| W | R | G | W | F | WRGWF_{T} |

TABLE 14
Examples of variants of SEQ ID NO: 21 (where
X₁ is L, X₂ is Q, X₃ is N, X₅ is S, X₆ is H)

| | | | | | Peptide |
|---|---|---|---|---|---|
| X₂₄ | X₂₅ | X₂₇ | X₂₉ | X₃₁ | Designation |
| S | K | S | Q | Y | SKSQY_{L} |
| S | K | S | Q | F | SKSQF_{L} |
| S | K | S | W | Y | SKSWY_{L} |
| S | K | S | W | F | SKSWF_{L} |
| S | K | G | Q | Y | SKGQY_{L} |
| S | K | G | Q | F | SKGQF_{L} |
| S | K | G | W | Y | SKGWY_{L} |
| S | K | G | W | F | SKGWF_{L} |
| S | L | S | Q | Y | SLSQY_{L} |
| S | L | S | Q | F | SLSQF_{L} |
| S | L | S | W | Y | SLSWY_{L} |
| S | L | S | W | F | SLSWF_{L} |
| S | L | G | Q | Y | SLGQY_{L} |
| S | L | G | Q | F | SLGQF_{L} |
| S | L | G | W | Y | SLGWY_{L} |
| S | L | G | W | F | SLGWF_{L} |
| S | R | S | Q | Y | SRSQY_{L} |
| S | R | S | Q | F | SRSQF_{L} |
| S | R | S | W | Y | SRSWY_{L} |
| S | R | S | W | F | SRSWF_{L} |
| S | R | G | Q | Y | SRGQY_{L} |
| S | R | G | Q | F | SRGQF_{L} |
| S | R | G | W | Y | SRGWY_{L} |
| S | R | G | W | F | SRGWF_{L} |
| W | K | S | Q | Y | WKSQY_{L} |
| W | K | S | Q | F | WKSQF_{L} |
| W | K | S | W | Y | WKSWY_{L} |
| W | K | S | W | F | WKSWF_{L} |
| W | K | G | Q | Y | WKGQY_{L} |
| W | K | G | Q | F | WKGQF_{L} |
| W | K | G | W | Y | WKGWY_{L} |
| W | K | G | W | F | WKGWF_{L} |
| W | L | S | Q | Y | WLSQY_{L} |
| W | L | S | Q | F | WLSQF_{L} |
| W | L | S | W | Y | WLSWY_{L} |
| W | L | S | W | F | WLSWF_{L} |
| W | L | G | Q | Y | WLGQY_{L} |
| W | L | G | Q | F | WLGQF_{L} |
| W | L | G | W | Y | WLGWY_{L} |
| W | L | G | W | F | WLGWF_{L} |
| W | R | S | Q | Y | WRSQY_{L} |
| W | R | S | O | F | WRSQF_{L} |
| W | R | S | W | Y | WRSWY_{L} |
| W | R | S | W | F | WRSWF_{L} |
| W | R | G | Q | Y | WRGQY_{L} |
| W | R | G | Q | F | WRGQF_{L} |
| W | R | G | W | Y | WRGWY_{L} |
| W | R | G | W | F | WRGWF_{L} |

TABLE 15
Examples of variants of SEQ ID NO: 21 (where
X₁ is V, X₂ is H, X₃ is N, X₅ is S, X₆ is H)

| | | | | | Peptide |
|---|---|---|---|---|---|
| X₂₄ | X₂₅ | X₂₇ | X₂₉ | X₃₁ | Designation |
| S | K | S | Q | Y | SKSQY_{VH} |
| S | K | S | Q | F | SKSQF_{VH} |
| S | K | S | W | Y | SKSWY_{VH} |
| S | K | S | W | F | SKSWF_{VH} |
| S | K | G | Q | Y | SKGQY_{VH} |
| S | K | G | Q | F | SKGQF_{VH} |
| S | K | G | W | Y | SKGWY_{VH} |
| S | K | G | W | F | SKGWF_{VH} |
| S | L | S | Q | Y | SLSQY_{VH} |
| S | L | S | Q | F | SLSQF_{VH} |
| S | L | S | W | Y | SLSWY_{VH} |
| S | L | S | W | F | SLSWF_{VH} |
| S | L | G | Q | Y | SLGQY_{VH} |
| S | L | G | Q | F | SLGQF_{VH} |
| S | L | G | W | Y | SLGWY_{VH} |
| S | L | G | W | F | SLGWF_{VH} |
| S | R | S | Q | Y | SRSQY_{VH} |
| S | R | S | Q | F | SRSQF_{VH} |
| S | R | S | W | Y | SRSWY_{VH} |
| S | R | S | W | F | SRSWF_{VH} |
| S | R | G | Q | Y | SRGQY_{VH} |
| S | R | G | Q | F | SRGQF_{VH} |
| S | R | G | W | Y | SRGWY_{VH} |
| S | R | G | W | F | SRGWF_{VH} |
| W | K | S | Q | Y | WKSQY_{VH} |
| W | K | S | Q | F | WKSQF_{VH} |
| W | K | S | W | Y | WKSWY_{VH} |
| W | K | S | W | F | WKSWF_{VH} |
| W | K | G | Q | Y | WKGQY_{VH} |
| W | K | G | Q | F | WKGQF_{VH} |
| W | K | G | W | Y | WKGWY_{VH} |
| W | K | G | W | F | WKGWF_{VH} |
| W | L | S | Q | Y | WLSQY_{VH} |
| W | L | S | Q | F | WLSQF_{VH} |
| W | L | S | W | Y | WLSWY_{VH} |
| W | L | S | W | F | WLSWF_{VH} |
| W | L | G | Q | Y | WLGQY_{VH} |
| W | L | G | Q | F | WLGQF_{VH} |
| W | L | G | W | Y | WLGWY_{VH} |
| W | L | G | W | F | WLGWF_{VH} |
| W | R | S | Q | Y | WRSQY_{VH} |
| W | R | S | Q | F | WRSQF_{VH} |
| W | R | S | W | Y | WRSWY_{VH} |
| W | R | S | W | F | WRSWF_{VH} |
| W | R | G | Q | Y | WRGQY_{VH} |
| W | R | G | Q | F | WRGQF_{VH} |
| W | R | G | W | Y | WRGWY_{VH} |
| W | R | G | W | F | WRGWF_{VH} |

TABLE 16
Examples of variants of SEQ ID NO: 21 (where
X₁ is T, X₂ is H, X₃ is N, X₅ is S, X₆ is H)

| | | | | | Peptide |
|---|---|---|---|---|---|
| X₂₄ | X₂₅ | X₂₇ | X₂₉ | X₃₁ | Designation |
| S | K | S | Q | Y | SKSQY_{TH} |
| S | K | S | Q | F | SKSQF_{TH} |
| S | K | S | W | Y | SKSWY_{TH} |
| S | K | S | W | F | SKSWF_{TH} |
| S | K | G | Q | Y | SKGQY_{TH} |
| S | K | G | Q | F | SKGQF_{TH} |
| S | K | G | W | Y | SKGWY_{TH} |
| S | K | G | W | F | SKGWF_{TH} |
| S | L | S | Q | Y | SLSQY_{TH} |
| S | L | S | Q | F | SLSQF_{TH} |
| S | L | S | W | Y | SLSWY_{TH} |
| S | L | S | W | F | SLSWF_{TH} |
| S | L | G | Q | Y | SLGQY_{TH} |
| S | L | G | Q | F | SLGQF_{TH} |
| S | L | G | W | Y | SLGWY_{TH} |
| S | L | G | W | F | SLGWF_{TH} |
| S | R | S | Q | Y | SRSQY_{TH} |
| S | R | S | Q | F | SRSQF_{TH} |
| S | R | S | W | Y | SRSWY_{TH} |
| S | R | S | W | F | SRSWF_{TH} |
| S | R | G | Q | Y | SRGQY_{TH} |
| S | R | G | Q | F | SRGQF_{TH} |
| S | R | G | W | Y | SRGWY_{TH} |
| S | R | G | W | F | SRGWF_{TH} |
| W | K | S | Q | Y | WKSQY_{TH} |
| W | K | S | Q | F | WKSQF_{TH} |
| W | K | S | W | Y | WKSWY_{TH} |
| W | K | S | W | F | WKSWF_{TH} |
| W | K | G | Q | Y | WKGQY_{TH} |
| W | K | G | Q | F | WKGQF_{TH} |
| W | K | G | W | Y | WKGWY_{TH} |
| W | K | G | W | F | WKGWF_{TH} |
| W | L | S | Q | Y | WLSQY_{TH} |
| W | L | S | Q | F | WLSQF_{TH} |
| W | L | S | W | Y | WLSWY_{TH} |
| W | L | S | W | F | WLSWF_{TH} |
| W | L | G | Q | Y | WLGQY_{TH} |
| W | L | G | Q | F | WLGQF_{TH} |
| W | L | G | W | Y | WLGWY_{TH} |
| W | L | G | W | F | WLGWF_{TH} |
| W | R | S | Q | Y | WRSQY_{TH} |
| W | R | S | Q | F | WRSQF_{TH} |
| W | R | S | W | Y | WRSWY_{TH} |
| W | R | S | W | F | WRSWF_{TH} |
| W | R | G | Q | Y | WRGQY_{TH} |
| W | R | G | Q | F | WRGQF_{TH} |
| W | R | G | W | Y | WRGWY_{TH} |
| W | R | G | W | F | WRGWF_{TH} |

TABLE 17
Examples of variants of SEQ ID NO: 21 (where
X₁ is L, X₂ is H, X₃ is N, X₅ is S, X₆ is H)

| | | | | | Peptide |
|---|---|---|---|---|---|
| X₂₄ | X₂₅ | X₂₇ | X₂₉ | X₃₁ | Designation |
| S | K | S | Q | Y | SKSQY_{LH} |
| S | K | S | Q | F | SKSQF_{LH} |
| S | K | S | W | Y | SKSWY_{LH} |
| S | K | S | W | F | SKSWF_{LH} |
| S | K | G | Q | Y | SKGQY_{LH} |
| S | K | G | Q | F | SKGQF_{LH} |
| S | K | G | W | Y | SKGWY_{LH} |
| S | K | G | W | F | SKGWF_{LH} |
| S | L | S | Q | Y | SLSQY_{LH} |
| S | L | S | Q | F | SLSQF_{LH} |
| S | L | S | W | Y | SLSWY_{LH} |
| S | L | S | W | F | SLSWF_{LH} |
| S | L | G | Q | Y | SLGQY_{LH} |
| S | L | G | Q | F | SLGQF_{LH} |
| S | L | G | W | Y | SLGWY_{LH} |
| S | L | G | W | F | SLGWF_{LH} |
| S | R | S | Q | Y | SRSQY_{LH} |
| S | R | S | Q | F | SRSQF_{LH} |
| S | R | S | W | Y | SRSWY_{LH} |
| S | R | S | W | F | SRSWF_{LH} |
| S | R | G | Q | Y | SRGQY_{LH} |
| S | R | G | Q | F | SRGQF_{LH} |
| S | R | G | W | Y | SRGWY_{LH} |
| S | R | G | W | F | SRGWF_{LH} |
| W | K | S | Q | Y | WKSQY_{LH} |
| W | K | S | Q | F | WKSQF_{LH} |
| W | K | S | W | Y | WKSWY_{LH} |
| W | K | S | W | F | WKSWF_{LH} |
| W | K | G | Q | Y | WKGQY_{LH} |
| W | K | G | Q | F | WKGQF_{LH} |
| W | K | G | W | Y | WKGWY_{LH} |
| W | K | G | W | F | WKGWF_{LH} |
| W | L | S | Q | Y | WLSQY_{LH} |
| W | L | S | Q | F | WLSQF_{LH} |
| W | L | S | W | Y | WLSWY_{LH} |
| W | L | S | W | F | WLSWF_{LH} |
| W | L | G | Q | Y | WLGQY_{LH} |
| W | L | G | Q | F | WLGQF_{LH} |
| W | L | G | W | Y | WLGWY_{LH} |
| W | L | G | W | F | WLGWF_{LH} |
| W | R | S | Q | Y | WRSQY_{LH} |
| W | R | S | Q | F | WRSQF_{LH} |
| W | R | S | W | Y | WRSWY_{LH} |
| W | R | S | W | F | WRSWF_{LH} |
| W | R | G | Q | Y | WRGQY_{LH} |
| W | R | G | Q | F | WRGQF_{LH} |
| W | R | G | W | Y | WRGWY_{LH} |
| W | R | G | W | F | WRGWF_{LH} |

TABLE 18
Examples of variants of SEQ ID NO: 21 (where
X₁ is V, X₂ is G, X₃ is N, X₅ is S, X₆ is H)

| | | | | | Peptide |
|---|---|---|---|---|---|
| X₂₄ | X₂₅ | X₂₇ | X₂₉ | X₃₁ | Designation |
| S | K | S | Q | Y | SKSQY_{VG} |
| S | K | S | Q | F | SKSQF_{VG} |
| S | K | S | W | Y | SKSWY_{VG} |
| S | K | S | W | F | SKSWF_{VG} |
| S | K | G | Q | Y | SKGQY_{VG} |
| S | K | G | Q | F | SKGQF_{VG} |
| S | K | G | W | Y | SKGWY_{VG} |
| S | K | G | W | F | SKGWF_{VG} |
| S | L | S | Q | Y | SLSQY_{VG} |
| S | L | S | Q | F | SLSQF_{VG} |
| S | L | S | W | Y | SLSWY_{VG} |
| S | L | S | W | F | SLSWF_{VG} |
| S | L | G | Q | Y | SLGQY_{VG} |
| S | L | G | Q | F | SLGQF_{VG} |
| S | L | G | W | Y | SLGWY_{VG} |
| S | L | G | W | F | SLGWF_{VG} |
| S | R | S | Q | Y | SRSQY_{VG} |
| S | R | S | Q | F | SRSQF_{VG} |
| S | R | S | W | Y | SRSWY_{VG} |
| S | R | S | W | F | SRSWF_{VG} |
| S | R | G | Q | Y | SRGQY_{VG} |
| S | R | G | Q | F | SRGQF_{VG} |
| S | R | G | W | Y | SRGWY_{VG} |
| S | R | G | W | F | SRGWF_{VG} |
| W | K | S | Q | Y | WKSQY_{VG} |
| W | K | S | Q | F | WKSQF_{VG} |
| W | K | S | W | Y | WKSWY_{VG} |
| W | K | S | W | F | WKSWF_{VG} |
| W | K | G | Q | Y | WKGQY_{VG} |
| W | K | G | Q | F | WKGQF_{VG} |
| W | K | G | W | Y | WKGWY_{VG} |
| W | K | G | W | F | WKGWF_{VG} |
| W | L | S | Q | Y | WLSQY_{VG} |
| W | L | S | Q | F | WLSQF_{VG} |
| W | L | S | W | Y | WLSWY_{VG} |
| W | L | S | W | F | WLSWF_{VG} |
| W | L | G | Q | Y | WLGQY_{VG} |
| W | L | G | Q | F | WLGQF_{VG} |
| W | L | G | W | Y | WLGWY_{VG} |
| W | L | G | W | F | WLGWF_{VG} |
| W | R | S | Q | Y | WRSQY_{VG} |
| W | R | S | Q | F | WRSQF_{VG} |
| W | R | S | W | Y | WRSWY_{VG} |
| W | R | S | W | F | WRSWF_{VG} |
| W | R | G | Q | Y | WRGQY_{VG} |
| W | R | G | Q | F | WRGQF_{VG} |
| W | R | G | W | Y | WRGWY_{VG} |
| W | R | G | W | F | WRGWF_{VG} |

TABLE 19
Examples of variants of SEQ ID NO: 21 (where
X₁ is T, X₂ is Q, X₃ is N, X₅ is S, X₆ is H)

| | | | | | Peptide |
|---|---|---|---|---|---|
| X₂₄ | X₂₅ | X₂₇ | X₂₉ | X₃₁ | Designation |
| S | K | S | Q | Y | SKSQY_{TG} |
| S | K | S | Q | F | SKSQF_{TG} |
| S | K | S | W | Y | SKSWY_{TG} |
| S | K | S | W | F | SKSWF_{TG} |
| S | K | G | Q | Y | SKGQY_{TG} |
| S | K | G | Q | F | SKGQF_{TG} |
| S | K | G | W | Y | SKGWY_{TG} |
| S | K | G | W | F | SKGWF_{TG} |
| S | L | S | Q | Y | SLSQY_{TG} |
| S | L | S | Q | F | SLSQF_{TG} |
| S | L | S | W | Y | SLSWY_{TG} |
| S | L | S | W | F | SLSWF_{TG} |
| S | L | G | Q | Y | SLGQY_{TG} |
| S | L | G | Q | F | SLGQF_{TG} |
| S | L | G | W | Y | SLGWY_{TG} |
| S | L | G | W | F | SLGWF_{TG} |
| S | R | S | Q | Y | SRSQY_{TG} |
| S | R | S | Q | F | SRSQF_{TG} |
| S | R | S | W | Y | SRSWY_{TG} |
| S | R | S | W | F | SRSWF_{TG} |
| S | R | G | Q | Y | SRGQY_{TG} |
| S | R | G | O | F | SRGQF_{TG} |
| S | R | G | W | Y | SRGWY_{TG} |
| S | R | G | W | F | SRGWF_{TG} |
| W | K | S | Q | Y | WKSQY_{TG} |
| W | K | S | Q | F | WKSQF_{TG} |
| W | K | S | W | Y | WKSWY_{TG} |
| W | K | S | W | F | WKSWF_{TG} |
| W | K | G | Q | Y | WKGQY_{TG} |
| W | K | G | Q | F | WKGQF_{TG} |
| W | K | G | W | Y | WKGWY_{TG} |
| W | K | G | W | F | WKGWF_{TG} |
| W | L | S | Q | Y | WLSQY_{TG} |
| W | L | S | Q | F | WLSQF_{TG} |
| W | L | S | W | Y | WLSWY_{TG} |
| W | L | S | W | F | WLSWF_{TG} |
| W | L | G | Q | Y | WLGQY_{TG} |
| W | L | G | Q | F | WLGQF_{TG} |
| W | L | G | W | Y | WLGWY_{TG} |
| W | L | G | W | F | WLGWF_{TG} |
| W | R | S | Q | Y | WRSQY_{TG} |
| W | R | S | Q | F | WRSQF_{TG} |
| W | R | S | W | Y | WRSWY_{TG} |
| W | R | S | W | F | WRSWF_{TG} |
| W | R | G | Q | Y | WRGQY_{TG} |
| W | R | G | Q | F | WRGQF_{TG} |
| W | R | G | W | Y | WRGWY_{TG} |
| W | R | G | W | F | WRGWF_{TG} |

TABLE 20
Examples of variants of SEQ ID NO: 21 (where
X₁ is L, X₂ is Q, X₃ is N, X₅ is S, X₆ is H)

| | | | | | Peptide |
|---|---|---|---|---|---|
| X₂₄ | X₂₅ | X₂₇ | X₂₉ | X₃₁ | Designation |
| S | K | S | Q | Y | SKSQY_{LG} |
| S | K | S | Q | F | SKSQF_{LG} |
| S | K | S | W | Y | SKSWY_{LG} |
| S | K | S | W | F | SKSWF_{LG} |
| S | K | G | Q | Y | SKGQY_{LG} |
| S | K | G | Q | F | SKGQF_{LG} |
| S | K | G | W | Y | SKGWY_{LG} |
| S | K | G | W | F | SKGWF_{LG} |
| S | L | S | Q | Y | SLSQY_{LG} |
| S | L | S | Q | F | SLSQF_{LG} |
| S | L | S | W | Y | SLSWY_{LG} |
| S | L | S | W | F | SLSWF_{LG} |
| S | L | G | Q | Y | SLGQY_{LG} |
| S | L | G | Q | F | SLGQF_{LG} |
| S | L | G | W | Y | SLGWY_{LG} |
| S | L | G | W | F | SLGWF_{LG} |
| S | R | S | Q | Y | SRSQY_{LG} |
| S | R | S | Q | F | SRSQF_{LG} |
| S | R | S | W | Y | SRSWY_{LG} |
| S | R | S | W | F | SRSWF_{LG} |
| S | R | G | Q | Y | SRGQY_{LG} |
| S | R | G | Q | F | SRGQF_{LG} |
| S | R | G | W | Y | SRGWY_{LG} |
| S | R | G | W | F | SRGWF_{LG} |
| W | K | S | Q | Y | WKSQY_{LG} |
| W | K | S | Q | F | WKSQF_{LG} |
| W | K | S | W | Y | WKSWY_{LG} |
| W | K | S | W | F | WKSWF_{LG} |
| W | K | G | Q | Y | WKGQY_{LG} |
| W | K | G | Q | F | WKGQF_{LG} |
| W | K | G | W | Y | WKGWY_{LG} |
| W | K | G | W | F | WKGWF_{LG} |
| W | L | S | Q | Y | WLSQY_{LG} |
| W | L | S | Q | F | WLSQF_{LG} |
| W | L | S | W | Y | WLSWY_{LG} |
| W | L | S | W | F | WLSWF_{LG} |
| W | L | G | Q | Y | WLGQY_{LG} |
| W | L | G | Q | F | WLGQF_{LG} |
| W | L | G | W | Y | WLGWY_{LG} |
| W | L | G | W | F | WLGWF_{LG} |
| W | R | S | Q | Y | WRSQY_{LG} |
| W | R | S | Q | F | WRSQF_{LG} |
| W | R | S | W | Y | WRSWY_{LG} |
| W | R | S | W | F | WRSWF_{LG} |
| W | R | G | Q | Y | WRGQY_{LG} |
| W | R | G | Q | F | WRGQF_{LG} |
| W | R | G | W | Y | WRGWY_{LG} |
| W | R | G | W | F | WRGWF_{LG} |

### DISCUSSION

For nearly two decades since its discovery AM2 (IMD) has been thought to be relatively non-selective agonist of the three CLR-RAMP complexes. Consistent with this, AM2 has many effects in common with CGRP and AM, so the rationale for the existence of a third agonist for the CGRP and AM receptors has been unclear. Similarly, the CGRP and AM₁ receptors have well defined roles in mediating key functions of CGRP and AM, respectively, but the reason for a second AM receptor, AM₂R, and its functions are poorly understood. Here, using real-time cAMP signaling and receptor binding assays and MD simulations we discovered that the AM2-AM₂R pairing has kinetic features that set it well apart from the eight other agonist-receptor combinations. Rather than being non-selective, AM2 is kinetically selective for the AM₂R at which it elicits a long-duration cAMP signaling phenotype. Results herein indicate that AM2 is the endogenous agonist of the fortuitously named AM₂R and indicate that the primary role of the AM₂R is to mediate long-duration AM2 signaling.

The discovery of the long duration signaling was facilitated by the real time cAMP biosensor assay in combination with the engineered high affinity peptide antagonists that we recently developed (J. M. Booe, M. L. Warner, A. A. Pioszak, Picomolar Affinity Antagonist and Sustained Signaling Agonist Peptide Ligands for the Adrenomedullin and Calcitonin Gene-Related Peptide Receptors. ACS pharmacology & translational science 3, 759-772 (2020)) (FIG. 1). This provided for straightforward characterization of the signaling duration capability of any agonist without the need for wash-out steps or labeled versions of each agonist. We reasoned that the signal decay rate after antagonist addition would be a proxy for agonist off-rate from the receptor. The NanoBRET™ direct binding assays bore this out by showing that although AM-TAMRA and AM-TAMRA had similar AM₂R binding affinities, AM2-TAMRA had slower on- and off-rates and consequently a longer residence time on the receptor (FIGS. 2A-2C, Table 7). The NanoBRET™ technology is powerful, but its limitations were evident in the association kinetics experiments where the calculated K_{D} values were ~10-fold lower than the equilibrium affinities. These discrepancies may reflect the signal decay issue, insufficient time resolution to rigorously define the early portion of the curves, and/or insufficient probe concentration range due to the decay being more pronounced at high probe concentrations. These issues may have prevented detection of the biphasic association curves expected for a 2-step (un)binding mechanism. Two other class B GPCR ligands, PTH and PTHrP, exhibited biphasic association curves for their receptor PTH1R using a different assay technology. Similar NanoBRET™ association kinetics discrepancies with equilibrium binding were reported in a study of relaxin binding to its receptor, which is thought to involve a multi-step mechanism.

Fortunately, in the NanoBRET™ dissociation kinetics experiments we could correct for signal decay, which revealed two-phase AM-TAMRA and AM2-TAMRA dissociation curves consistent with a 2-step (un)binding mechanism. The fast unbinding component may arise from peptide-receptor complexes in which the peptide is engaged solely to the receptor ECD and the slow unbinding component may come from complexes in which the peptide is fully engaged to both ECD and TMD. This interpretation is consistent with a recent cryo-EM structure of the CGRP-bound CGRP receptor in the absence of G protein. This showed that CGRP was primarily engaged solely to the receptor ECD, with a fraction of complexes showing limited engagement of the TMD by CGRP. Here, the fast dissociation components for AM-TAMRA and AM2-TAMRA accounted for 66.5% and 34.7% of their curves, respectively, which implies that AM2-TAMRA had a greater capacity to fully engage the uncoupled AM₂R than AM-TAMRA. This may in part explain the greater thermal stability of AM2-AM₂R observed in the native PAGE assay (FIG. 3(D)). Adding the fast and slow dissociation component half-lives multiplied by their fractional contributions yields combined half-life estimates of 2.8 min and 52 min for AM-TAMRA and AM2-TAMRA, respectively, which are close to the cAMP signaling decay half-lives obtained for these probes in the CAMYEL assay (FIG. 11B(A,B)). These are good agreements considering that the binding assay with uncoupled AM₂R in membranes is a simplification as compared to the cell-based signaling assay. Notably, the native PAGE coupling assays indicated that AM2 also had a slower off-rate from the G protein-coupled AM₂R (FIG. 3(B, C)). Overall, the NanoBRET™ and native PAGE assays were consistent with the longer residence time of AM2 at AM₂R being responsible for its longer duration cAMP signaling capability.

Using AM and AM2 chimeras we mapped the region responsible for the slow off-rate to the 11 amino acid R23-R33 segment of AM2 that binds at the interface of the CLR ECD and TMD (FIGS. 5A-5D). This encompasses the end of the AM2 α-helix and the hinge. The MD simulations revealed a series of polar interactions stabilizing this segment and its interactions with CLR. These are anchored at one end by the strong intermolecular R23-CLR D96 salt-bridge and at the other by a series of H-bonds involving R33 including intermolecular bonds to the loop at the base of the CLR ECD, and intramolecular bonds to the backbones of P30 in the hinge and M28 at the end of the helix. Neither of these anchoring interactions alone was sufficient to confer the slow off-rate as revealed by the AM-AM2 half and AM2-AM half chimeras. The corresponding central segment of AM exhibited much less stability in the MD simulations, thus providing a structural basis for the different off-rates of the two peptides. AM lacks an R23 equivalent so it cannot form the salt-bridge and its R33 equivalent is K38, which cannot form the same pattern of H-bonds due its smaller side chain and the AM hinge being one amino acid shorter than the AM2 hinge. A recent cryo-EM study of the amylin receptors, which are RAMP complexes with the related calcitonin receptor, indicated that a central 7 amino acid segment of the amylin peptide, which the authors termed the “bypass motif,” contributed to amylin receptor selectivity. These results make it clear that the mid-regions of the calcitonin/CGRP family peptides are not simply passive linkers connecting their TMD- and ECD-binding segments.

Interestingly, the AM2-AM ECD chimera appeared to exhibit a gain-of-function slower signaling decay at AM₂R as compared to AM2, although this did not reach statistical significance. This indicated that the AM ECD-binding fragment might work better with AM2 R23-R33 than the AM2 ECD binding fragment. This was unexpected because AM2 binds the purified AM₂R ECD complex with higher affinity than AM. The basis for this result is unclear, but it might involve the difference at the equivalent AM N40 and AM2 D35 positions because the AM2-TAMRA probe, which contained the D35K substitution, exhibited a similar gain-of-function. The AM-AM2-AM chimera had a gain-of-function slow decay at AM₂R that was significantly different than AM2, indicating that the AM N-terminal region works better with AM2 R23-R33. Remarkably, the AM2-AM ECD and AM-AM2-AM chimeras both exhibited a gain-of-function slow signaling decay phenotype at the AM₁R, with AM-AM2-AM being particularly noteworthy. Yet none of the AM/AM2 peptides-wild type or chimeras-exhibited slow signaling decay capacity at the CGRPR. This can possibly be understood by considering the orientations of the CLR ECD relative to the TMD that appear to be stabilized by each RAMP. RAMP2 and RAMP3 appeared to promote similar CLR ECD-TMD arrangements such that the R23-R33 segment could form interactions in AM₁R like those observed in AM₂R. In contrast, RAMP1 appears to promote a different ECD-TMD arrangement that would prevent the R23-R33 segment from making the same pattern of interactions. These different CLR ECD-TMD arrangements were attributed to the different RAMP linker sequences that connect the ECD and TM helix and were proposed to be a contributor to ligand selectivity. Our data are consistent with this for the CGRP receptor vs. the two AM receptors, but the RAMP linkers do not control peptide selectivity among the two AM receptors (see below discussion of RAMP chimeras and mutants).

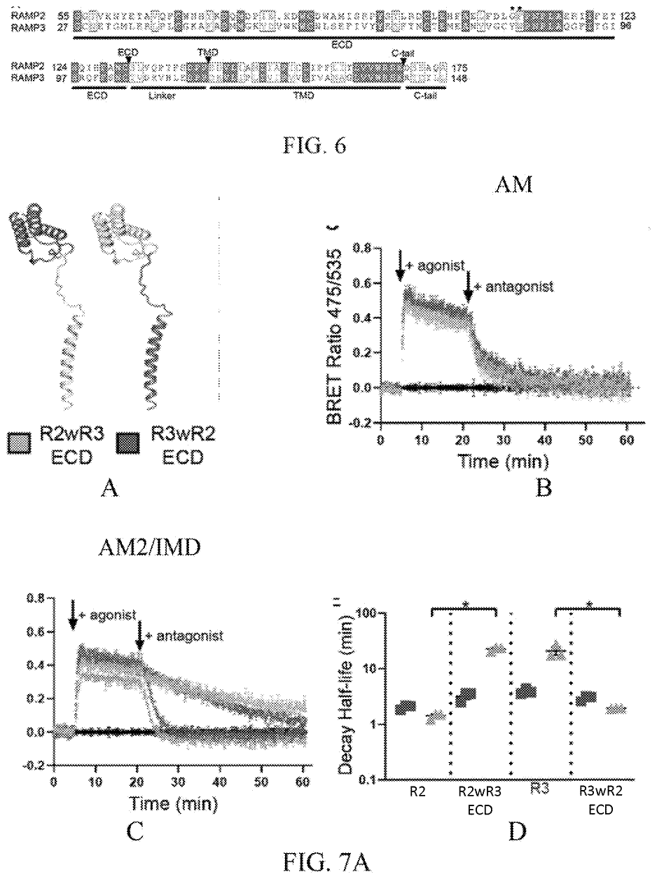

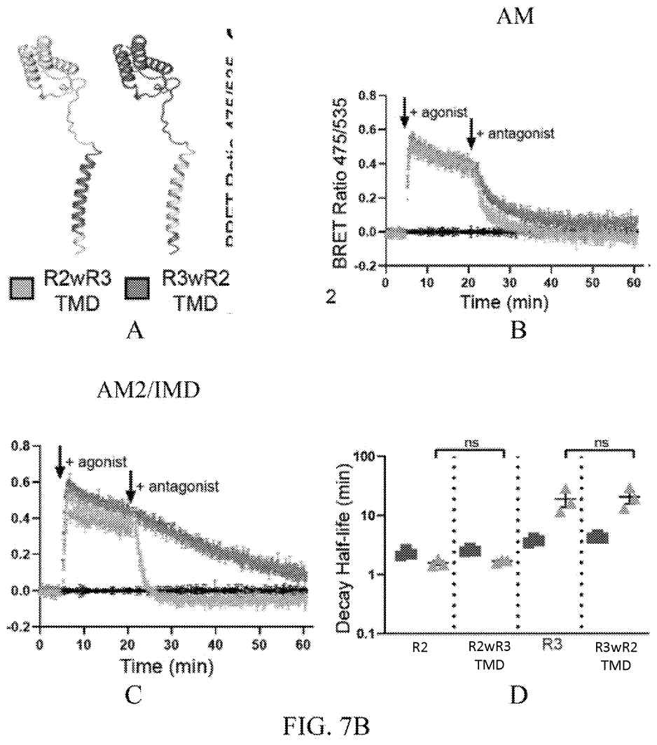

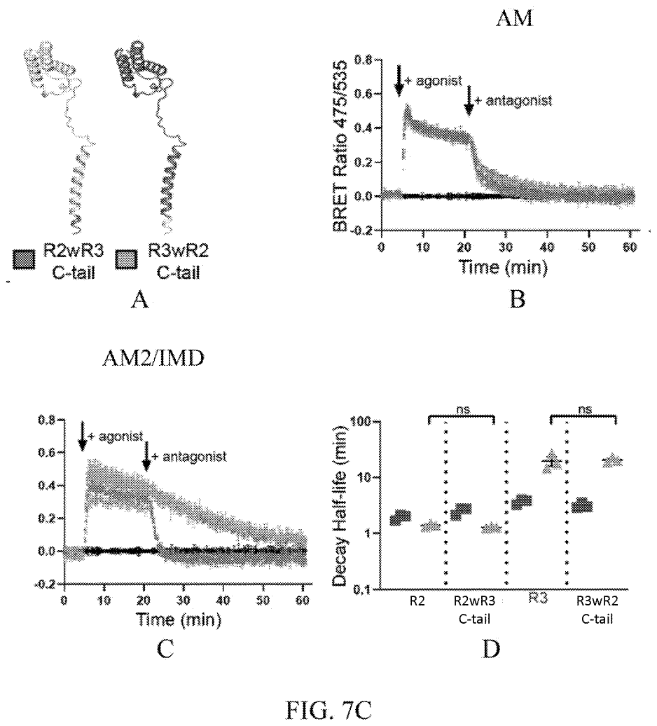

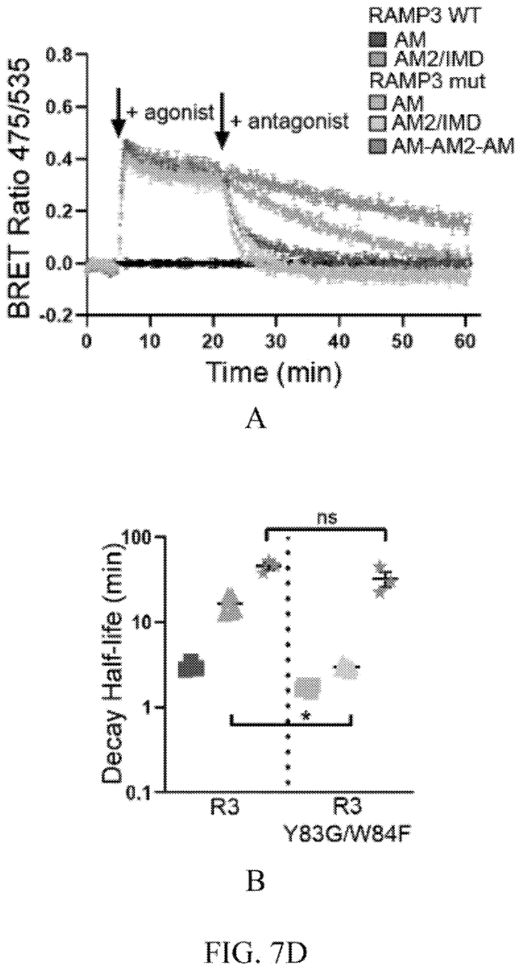

Why is AM2 kinetically selective for AM₂R if its R23-R33 segment has the capacity to confer slow decay at AM₁R? This was explored through RAMP2/3 chimeras, RAMP3 mutants, and the MD simulations (FIGS. 6 and 7A-7D). The chimeras unequivocally showed that the RAMP3 ECD enabled the AM2 slow signaling decay, and the RAMP3 Y83G/W84F swap mutant indicated that this was due to its role in forming the binding pocket for the peptide C-terminus. The simulations revealed a more stable AM2-AM₂R ECD complex as compared to the AM-bound version, particularly for the peptide β-turn and C-terminus near RAMP3 Y83 and W84. A network of water-mediated H-bonds in the pocket involved AM2 Y47 and RAMP3 E74 and is stabilized by hydrogen bonds with the RAMP3 W84 backbone as well as an intramolecular RAMP3 W84-T73 hydrogen bond. In contrast, AM binding was more dependent on the AM K46-RAMP3 E74 salt-bridge, consistent with prior mutagenesis studies. RAMP2 can also form this salt-bridge via E101, but G110 and F111 are unable to provide the optimal environment for the AM2 C-terminus. This explains why AM has nearly equal affinities for the purified AM₁R and AM₂R ECD complexes, whereas AM2 has higher affinity for the AM₂R ECD complex. Hence, the slow decay phenotype of AM2 was lost at the AM₂R with RAMP3 Y83G/W84F, whereas the AM-AM2-AM chimera retained slow decay at the mutant AM₂R. These results highlight the significant contribution of the CLR-RAMP ECD complexes to ligand selectivity, which is not surprising because ECD-binding is likely the first step in the peptide binding mechanism and the purified CLR-RAMP1/2/3 ECD complexes exhibited peptide selectivity profiles similar to the those of the intact receptors. Kinetic selectivity of AM2 for AM₂R is thus a two-step process beginning with its preference for the AM₂R ECD over the AM₁R ECD, after which its mid-region R23-R33 segment enables the stable interactions at the ECD-TMD interface that yield the slow off-rate. AM2 binds the CGRPR ECD with the same affinity as the AM₂R ECD, however, its mid-region fails to elicit a slow off-rate at the CGRPR, possibly for reasons described above. Our results emphasize the concept that the peptide agonists and the RAMP accessory proteins can collaborate to dictate CLR pharmacology. The RAMPs play a substantial role in determining receptor phenotype through a combination of their augmentation of the CLR ECD binding site and their modulation of CLR ECD-TMD inter-domain arrangement and/or dynamics, but unique agonist properties can further shape the signaling outcomes as shown here for AM2.

The pharmacology of AM2 and AM at the AM₂R is reminiscent of the behavior of the peptides PTH and PTHrP at their shared class B GPCR, the parathyroid hormone receptor PTH1R, which regulates calcium homeostasis and bone development. PTH and PTHrP are equipotent agonists of the PTH1R, but the endocrine hormone PTH exhibits a significantly slower PTH1R off-rate and longer duration cAMP signaling than the paracrine factor PTHrP. The basis for their different off-rates was mapped to a single residue difference near the N-terminus of the peptides that binds deep within the PTH1R TMD, so despite the conceptual similarities between the two systems, PTH and AM2 use very different structural mechanisms to attain their slow off-rates. The long receptor residence time of PTH allows it to continue to direct cAMP signaling from internalized PTH1R in endosomes, whereas PTHrP is limited to a traditional cAMP signal at the plasma membrane. These spatiotemporal differences are thought to underlie the different PTH and PTHrP biology and analogs designed to exploit these different kinetic properties have been developed as potential therapeutics for bone disorders. AM2 might similarly direct AM₂R signaling from endosomes because agonist stimulated AM₂R undergoes internalization, however, AM₂R trafficking can be further complicated by RAMP3 interaction with cytosolic factors that alter its trafficking, in some cases by blocking internalization.

In further aspects, the embodiments of the present disclosure include, but are not limited to, variants and variant fragments and chimeras of wild-type human AM, AM2, CGRP, AMY, and CT peptides which have higher affinity than the homologous wild type peptides for at least one of, two of, or all three of the CLR:RAMP1, CLR:RAMP2, or CLR:RAMP3 receptor complexes, wherein higher affinity means at least 50-fold, at least 100-fold, at least 200-fold, at least 300-fold, at least 400-fold, at least 500-fold, at least 600-fold, at least 700-fold, at least 800-fold, at least 900-fold, at least 1000-fold, at least 1100-fold, at least 1200-fold, at least 1300-fold, at least 1400-fold, at least 1500-fold, at least 1600-fold, at least 1700-fold, at least 1800-fold, at least 1900-fold, at least 2000-fold, at least 2500-fold, at least 3000-fold, at least 3500-fold, at least 4000-fold, at least 4500-fold, at least 5000-fold, at least 5500-fold, at least 6000-fold, at least 6500-fold, at least 7000-fold, at least 7500-fold, at least 8000-fold, at least 8500-fold, at least 9000-fold, at least 9500-fold, at least 10000-fold, at least 15000-fold, or at least 20000-fold, or greater, affinity, for at least one of, two of, or all three of the CLR:RAMP1, CLR:RAMP2, or CLR:RAMP3 receptor complexes. The variant and chimeric peptides of the present disclosure may have affinity Kᵢ's which are <1 μM, <750 nM, <500 nM, <400 nM, <300 nM, <200 nM, <100 nM, <50 nM, <10 nM, <1 nM, <100 pM, <10 pM, <5 pM, <2 pM, or <1 pM when binding to the receptor complex CLR:RAMP1, CLR:RAMP2, and/or CLR:RAMP3 is measured.

### NON-LIMITING ILLUSTRATIVE EMBODIMENTS

Illustrative embodiment 1. A peptide, comprising an amino acid sequence selected from the group consisting of SEQ ID NOS:51 and 87-103.

Illustrative embodiment 1A. The peptide of Illustrative embodiment 1, wherein the peptide comprises an N-terminal blocking group.

Illustrative embodiment 2. The peptide of Illustrative embodiment 1A, wherein the N-terminal blocking group is a lipid or an acetyl such that the N-terminal amino acid is lipidated or acetylated, respectively.

Illustrative embodiment 3. The peptide of claim 2, wherein the N-terminal blocking group is a lipid.

Illustrative embodiment 3A. The peptide of Illustrative embodiment 2, wherein the N-terminal blocking group is an acetyl.

Illustrative embodiment 4. The peptide of any of Illustrative embodiments 1-3A, wherein the peptide is directly bound to a carrier molecule or indirectly bound to a carrier molecule via a linker molecule.

Illustrative embodiment 5. The peptide of any of Illustrative embodiments 1-4, having agonistic activity or antagonistic activity for the at least one receptor complex.

Illustrative embodiment 6. The peptide of any of Illustrative embodiments 1-5, wherein the peptide consists of up to 70 amino acids.

Illustrative embodiment 7. The peptide of any of Illustrative embodiments 1-6, comprising an affinity Kᵢ for receptor complex CLR:RAMP3 of less than 1 μM.

Illustrative embodiment 8. The peptide of Illustrative embodiment 7, wherein the affinity Kᵢ is less than 500 nM.

Illustrative embodiment 9. A peptide, comprising: an amino acid sequence selected from the group consisting of SEQ ID NOS:53, 56, and 104-119.

Illustrative embodiment 9A. The peptide of Illustrative embodiment 9, wherein the peptide comprises an N-terminal blocking group.

Illustrative embodiment 10. The peptide of claim 9A, wherein the N-terminal blocking group is a lipid or an acetyl such that the N-terminal amino acid is lipidated or acetylated, respectively.

Illustrative embodiment 11. The peptide of claim 10, wherein the N-terminal blocking group is a lipid.

Illustrative embodiment 11A. The peptide of Illustrative embodiment 10, wherein the N-terminal blocking group is an acetyl.

Illustrative embodiment 12. The peptide of any of Illustrative embodiments 9-11A, wherein the peptide is directly bound to a carrier molecule or indirectly bound to a carrier molecule via a linker molecule.

Illustrative embodiment 13. The peptide of any of Illustrative embodiments 9-12, having agonistic activity or antagonistic activity for the at least one receptor complex.

Illustrative embodiment 14. The peptide of any of Illustrative embodiments 9-13, wherein the peptide consists of up to 70 amino acids.

Illustrative embodiment 15. The peptide of any of Illustrative embodiments 9-14, comprising an affinity Kᵢ for receptor complex CLR:RAMP3 of less than 1 μM.

Illustrative embodiment 15A. The peptide of Illustrative embodiment 15, wherein the affinity Kᵢ is less than 500 nM.

Illustrative embodiment 16. A method of treating a subject for a condition regulated by a calcitonin receptor-like receptor-receptor activity-modifying protein 3 (CLR:RAMP3) receptor complex, the method comprising the step of: administering to the subject in need of such therapy an effective amount of a peptide comprising an amino acid sequence selected from the group consisting of SEQ ID NOS:51, 53, 56, and 87-119.

Illustrative embodiment 16A. The method of Illustrative embodiment 16, wherein the peptide comprises an N-terminal blocking group.

Illustrative embodiment 17. The method of Illustrative embodiment 16A, wherein the N-terminal blocking group is a lipid or an acetyl such that the N-terminal amino acid is lipidated or acetylated, respectively.

Illustrative embodiment 17A. The method of Illustrative embodiment 17, wherein the N-terminal blocking group is a lipid.

Illustrative embodiment 17B. The method of Illustrative embodiment 17, wherein the N-terminal blocking group is an acetyl.

Illustrative embodiment 18. The method of any of Illustrative embodiments 16-17B, wherein the peptide has agonistic activity or antagonistic activity for the at least one receptor complex.

Illustrative embodiment 18A. The method of any of Illustrative embodiments 16-18, wherein the peptide is directly bound to a carrier molecule or indirectly bound to a carrier molecule via a linker molecule.

Illustrative embodiment 19. The method of any of Illustrative embodiments 16-18A, wherein the peptide consists of up to 70 amino acids.

Illustrative embodiment 20. The method of any of Illustrative embodiments 16-19, wherein the peptide comprises an affinity Kᵢ for receptor complex CLR:RAMP3 of less than 1 μM.

Illustrative embodiment 21. The peptide of Illustrative embodiment 20, wherein the affinity Kᵢ is less than 500 nM.

While the present disclosure has been described in connection with certain non-limiting embodiments of variant and chimeric peptide compounds, conjugates, compositions, and methods of production and application thereof so that aspects thereof may be more fully understood and appreciated, it is not intended that the present disclosure be limited to these particular embodiments. On the contrary, it is intended that all alternatives, modifications and equivalents are included within the scope of the present disclosure. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the presently disclosed methods and compositions. Changes may be made in the formulation of the various compositions described herein, the methods described herein or in the steps or the sequence of steps of the methods described herein without departing from the spirit and scope of the present disclosure.

What is claimed is:

1. A peptide, comprising:
an amino acid sequence selected from the group consisting of SEQ ID NOS:51 and 87-103, wherein the peptide optionally comprises an N-terminal blocking group.

2. The peptide of claim 1, wherein the optional N-terminal blocking group is a lipid or an acetyl such that the N-terminal amino acid is lipidated or acetylated, respectively.

3. The peptide of claim 1, wherein the optional N-terminal blocking group is a lipid.

4. The peptide of claim 1, wherein the peptide is directly bound to a carrier molecule or indirectly bound to a carrier molecule via a linker molecule.

5. The peptide of claim 1, having agonistic activity or antagonistic activity for the at least one receptor complex.

6. The peptide of claim 1, wherein the peptide consists of up to 70 amino acids.

7. The peptide of claim 1, comprising an affinity Kᵢ for receptor complex CLR:RAMP3 of less than 1 μM.

8. The peptide of claim 7, wherein the affinity Kᵢ is less than 500 nM.

9. A peptide, comprising:
an amino acid sequence selected from the group consisting of SEQ ID NOS:53, 56, and 104-119, wherein the peptide optionally comprises an N-terminal blocking group.

10. The peptide of claim 9, wherein the optional N-terminal blocking group is a lipid or an acetyl such that the N-terminal amino acid is lipidated or acetylated, respectively.

11. The peptide of claim 10, wherein the optional N-terminal blocking group is a lipid.

12. The peptide of claim 9, wherein the peptide is directly bound to a carrier molecule or indirectly bound to a carrier molecule via a linker molecule.

13. The peptide of claim 9, having agonistic activity or antagonistic activity for the at least one receptor complex.

14. The peptide of claim 9, wherein the peptide consists of up to 70 amino acids.

15. The peptide of claim 9, comprising an affinity Kᵢ for receptor complex CLR:RAMP3 of less than 1 μM.

16. A method of treating a subject for a condition regulated by a calcitonin receptor-like receptor-receptor activity-modifying protein 3 (CLR:RAMP3) receptor complex, the method comprising the step of:
administering to the subject in need of such therapy an effective amount of a peptide comprising an amino acid sequence selected from the group consisting of SEQ ID NOS:51, 53, 56, and 87-119, wherein the peptide optionally comprises an N-terminal blocking group.

17. The method of claim 16, wherein the optional N-terminal blocking group is a lipid or an acetyl such that the N-terminal amino acid is lipidated or acetylated, respectively.

18. The method of claim 16, wherein the peptide has agonistic activity or antagonistic activity for the at least one receptor complex.

19. The method of claim 16, wherein the peptide consists of up to 70 amino acids.

20. The method of claim 16, wherein the peptide comprises an affinity Kᵢ for receptor complex CLR:RAMP3 of less than 1 μM.
